(19) United States
(12) Patent Application Publication
(10) Pub. No.: US 2019/0284298 A1
(43) Pub. Date: Sep. 19, 2019

(54) USE OF ANTIBODY-COUPLED T CELL RECEPTOR (ACTR) WITH MULTIPLE ANTI-CANCER ANTIBODIES IN CANCER TREATMENT

(71) Applicant: Unum Therapeutics Inc., Cambridge, MA (US)

(72) Inventors: Seth Ettenberg, Cambridge, MA (US); Heather Huet, Cambridge, MA (US); Eugene Choi, Cambridge, MA (US); Casey Judge, Cambridge, MA (US); Gregory Motz, Cambridge, MA (US)

(73) Assignee: Unum Therapeutics Inc., Cambridge, MA (US)

(21) Appl. No.: 16/318,566

(22) PCT Filed: Jul. 19, 2017
(86) PCT No.: PCT/US17/42730

§ 371 Date: Jan. 17, 2019

Related U.S. Application Data
(60) Provisional application No. 62/429,478, filed on Dec. 2, 2016.
(60) Provisional application No. 62/410,925, filed on Oct. 21, 2016.
(60) Provisional application No. 62/370,820, filed on Aug. 4, 2016.
(60) Provisional application No. 62/364,213, filed on Jul. 19, 2016.

(51) Int. Cl. C07K 16/32; C07K 16/28; C07K 14/715; C07K 14/725; C07K 14/705; C07K 14/735; A61K 39/395; A61P 35/00; G01N 33/50

(52) U.S. Cl. CPC C07K 16/32; C07K 2317/53; C07K 14/7151; C07K 14/7051; C07K 14/70596; C07K 16/2803; C07K 16/283; C07K 16/2896; C07K 16/2887; C07K 14/70535; A61K 39/39558; A61P 35/00; G01N 33/5011; C07K 2319/30; C07K 2319/02; C07K 2319/03; C07K 2317/41; C07K 2317/24; C07K 2317/21; C07K 16/2863

(57) ABSTRACT

Disclosed herein are methods of using immune cells expressing chimeric receptors and two or more anti-cancer antibodies that bind cancer antigens in cancer therapy.

### CROSS REFERENCE TO RELATED APPLICATIONS

This application claims the benefit of the filing date of U.S. Provisional Application No. 62/364,213, filed Jul. 19, 2016, U.S. Provisional Application No. 62/370,820, filed Aug. 4, 2016, U.S. Provisional Application No. 62/410,925, filed Oct. 21, 2016, and U.S. Provisional Application No. 62/429,478, filed Dec. 2, 2016. The entire contents of each of these referenced applications are incorporated by reference herein.

### BACKGROUND OF THE INVENTION

Cancer immunotherapy, including cell-based therapy, antibody therapy and cytokine therapy, is used to provoke immune responses attacking tumor cells while sparing normal tissues. It is a promising option for treating various types of cancer because of its potential to evade genetic and cellular mechanisms of drug resistance, and to target tumor cells while sparing normal tissues. T-lymphocytes can exert major anti-tumor effects as demonstrated by results of allogeneic hematopoietic stem cell transplantation (HSCT) for hematologic malignancies, where T-cell-mediated graft-versus-host disease (GvHD) is inversely associated with disease recurrence, and immunosuppression withdrawal or infusion of donor lymphocytes can contain relapse. Weiden et al., N Engl J Med. 1979; 300(19):1068-1073; Porter et al., N Engl J Med. 1994; 330(2):100-106; Kolb et al., Blood. 1995; 86(5):2041-2050; Slavin et al., Blood. 1996; 87(6):2195-2204; and Appelbaum, Nature. 2001; 411(6835):385-389.

Cell-based therapy may involve cytotoxic T cells having reactivity skewed toward cancer cells. Eshhar et al., Proc. Natl. Acad. Sci. U.S.A.; 1993; 90 (2):720-724; Geiger et al., J Immunol. 1999; 162(10):5931-5939; Brentjens et al., Nat. Med. 2003; 9(3):279-286; Cooper et al., Blood. 2003; 101(4):1637-1644; and Imai et al., Leukemia. 2004; 18:676-684. One approach is to express a chimeric antigen receptor having an antigen-binding domain fused to one or more T cell activation signaling domains. Binding of a cancer antigen via the antigen-binding domain results in T cell activation and triggers cytotoxicity. Recent results of clinical trials with infusions of chimeric receptor-expressing autologous T lymphocytes provided compelling evidence of their clinical potential. Pule et al., Nat. Med. 2008; 14(11):1264-1270; Porter et al., N Engl J Med; 2011; 25; 365(8):725-733; Brentjens et al., Blood. 2011; 118(18):4817-4828; Till et al., Blood. 2012; 119(17):3940-3950; Kochenderfer et al., Blood. 2012; 119(12):2709-2720; and Brentjens et al., Sci Transl Med. 2013; 5(177):177ra138.

Antibody-based immunotherapies, such as monoclonal antibodies, antibody-fusion proteins, and antibody drug conjugates (ADCs) are used to treat a wide variety of diseases, including many types of cancer. Such therapies may depend on recognition of cell surface molecules that are differentially expressed on cells for which elimination is desired (e.g., target cells such as cancer cells) relative to normal cells (e.g., non-cancer cells). Binding of an antibody-based immunotherapy to a cancer cell can lead to cancer cell death via various mechanisms, e.g., antibody-dependent cell-mediated cytotoxicity (ADCC), complement-dependent cytotoxicity (CDC), or direct cytotoxic activity of the payload from an antibody-drug conjugate (ADC).

### SUMMARY OF THE INVENTION

The present disclosure is based, at least in part, on the unexpected discovery that cancer antigens can be effectively targeted by immune cells such as T cells expressing a surface antibody-coupled T cell receptor (ACTR) when co-used with at least two anti-cancer antibodies, which is greater than targeting with any single anti-cancer antibody. In particular, the co-use of at least two anti-cancer antibodies together with T cells expressing an exemplary ACTR construct (ACTR-T cells) successfully targeted cancer cells expressing very low level of a cancer antigen (e.g., HER2 in cancer cells not amplified with HER2 gene), while such anti-cancer effects were not observed when only one anti-cancer antibody was co-used with ACTR-T cells. Thus, the co-use of immune cells expressing such ACTR and at least two anti-cancer antibodies would be effective in inhibiting such cancer cells, which are known to be difficult to target using conventional therapies.

Accordingly, one aspect of the present disclosure provides a method of treating cancer in a subject, the method comprising administering to a subject in need thereof a therapeutically effective amount of an immune cell that expresses a surface chimeric receptor (an ACTR), and a therapeutically effective amount of at least two anti-cancer antibodies. The chimeric receptor comprises at least an Fc binding domain and a cytoplasmic signaling domain, and optionally a transmembrane domain, a co-stimulatory signaling domain, a hinge domain, or a combination thereof.

In some embodiments, the chimeric receptor comprises, from N-terminus to C-terminus, an Fc binding domain, a transmembrane domain, a co-stimulatory domain, and a cytoplasmic signaling domain. In one example, the chimeric receptors described herein may further comprise a hinge domain, which can be located between the Fc binding domain and the transmembrane domain. When needed, the chimeric receptor may further comprise a signal peptide, which may be located at the N-terminus of the receptor.

In some embodiments, the Fc binding domain of any of the chimeric receptors described herein may comprise an extracellular domain of an Fc receptor, which can be a Fcγ receptor (FcγR). Examples include, but are not limited to, CD16A, CD16B, CD64A, CD64B, CD64C, CD32A, and CD32B. Alternatively or in addition, the chimeric receptor comprises a co-stimulatory domain of 4-1BB or CD28. The chimeric receptor described herein may comprise a cytoplasmic signaling domain of CD3ζ.

In some examples, the chimeric receptor comprises an extracellular domain of CD16A, a co-stimulatory domain of 4-1BB or CD28, and a cytoplasmic signaling domain of CD3ζ. Such chimeric receptors may further comprise a hinge domain of CD8a or CD28. In specific examples, the chimeric receptor may comprise the amino acid sequence SEQ ID NO: 1 or SEQ ID NO:67.

In some embodiments, the at least two anti-cancer antibodies bind to two different cancer antigens. In other embodiments, the at least two anti-cancer antibodies bind to different epitopes of the same cancer antigen. In some embodiments, the cancer antigen(s) can be HER2, EGFR, BCMA, CD19, CD20, CD22, CD38, and/or CS1. In some examples, the cancer antigen is HER2 and one or both of the at least two anti-cancer antibodies are anti-HER2 antibodies, for example, the at least two anti-cancer antibodies can be Trastuzumab and Pertuzumab. In some examples, the cancer antigen is EGFR and one or both of the at least two anti-cancer antibodies can be any anti-EGFR antibodies known in the art including, but not limited to: cetuximab, panitumumab, MM-151, SCT200, GC1118, Necitumumab, Nimotuzumab, anti-EGFR antibody 992, anti-EGFR antibody 1024, or Zalutumumab. In some embodiments, the cancer antigens are CD19 and/or CD22 and one of the at least two anti-cancer antibodies is an anti-CD19 antibody or an anti-CD22 antibody. In some instances, the at least two anti-cancer antibodies can be an anti-CD19 antibody and an anti-CD22 antibody, for example, MOR208 and Epratuzumab. In some embodiments, the cancer antigen(s) is CD38 and/or CS1 and one of the at least two anti-cancer antibodies is an anti-CD38 antibody or an anti-CS1 antibody. In some instances, the at least two antibodies are an anti-CD38 antibody and an anti-CS1 antibody, for example, Daratumumab and Elotuzumab. In some embodiments, the cancer antigens are BCMA and CD38. In some embodiments, the cancer antigens are BCMA and CS1. In some embodiments, the cancer antigens are CD19 and CD20. In some embodiments, the cancer antigens are CD22 and CD20. In some embodiments, the cancer is of a tissue selected from the group consisting of breast, haematopoeitic cells, large intestine, liver, lung, ovary, salivary gland, skin, stomach, and aerodigestive tract.

In some embodiments, the subject is a human cancer patient who has cancer cells that express a low level of the cancer antigen, to which the at least two antibodies target. For example, the human cancer patient may have cancer cells that express a low level of HER2 or EGFR. In some instances, the human cancer patient may have HER2 non-amplified cancer cells.

In another aspect, the present disclosure provides a kit comprising (a) any of the immune cells described herein, which express the chimeric receptor also described herein, and (b) and at least two anti-cancer antibodies.

In another aspect, the present disclosure provides in vitro method for evaluating the activity of an immune cell that expresses a chimeric receptor, the method comprising: (a) incubating the immune cell expressing the chimeric receptor with a first target cell in the presence of a first agent that comprises an Fc domain, wherein the chimeric receptor comprises an Fc binding domain, and a cytoplasmic signaling domain; and wherein the first agent binds a surface receptor of the first target cell; (b) measuring cytotoxicity of the first target cell induced by the immune cell in the presence of the first agent; (c) separating the immune cell from the first target cell and the first agent; (d) incubating the immune cell obtained in step (c) with a second target cell in the presence of a second agent that comprises an Fc domain, wherein the second agent binds a surface receptor of the second target cell; and (e) measuring cytotoxicity of the second target cell induced by the immune cell in the presence of the second agent.

In some embodiments, the first agent, the second agent, or both are antibodies. In certain embodiments, the first agent and the second agent are antibodies specific to different cell surface receptors. In some embodiments, the first target cell, the second target cell, or both are cancer cells. In certain embodiments, the chimeric receptor further comprises a signal domain from a co-stimulatory receptor.

Also within the scope of the present disclosure are (a) pharmaceutical compositions for use in treating cancer, the pharmaceutical composition comprising immune cells as described herein that express any of the chimeric receptor constructs described herein and at least wo anti-cancer antibodies; and (b) use of such immune cells and anti-cancer antibodies for manufacturing a medicament for use in the intended treatment.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIG. 1 is a graph showing the maximum cell binding of the indicated anti-HER2 antibody or combination of anti-HER2 antibodies on the x-axis relative to the maximum activation of ACTR-expressing Jurkat cells, as measured by an NFAT-luciferase reporter construct, in combination with the indicated antibody or combination of antibodies on the y-axis. Each circle represents a different cancer tissue, and the size of the circle indicates the relative expression of HER2 on the cells of the cancer. Panel A: Pertuzumab with ACTR-expressing Jurkat cells. Panel B: Trastuzumab with ACTR-expressing Jurkat cells. Panel C: Pertuzumab and Trastuzumab in combination with ACTR-expressing Jurkat cells.

FIG. 2 is a graph showing the maximum cell binding of the indicated anti-EGFR antibody or combination of anti-EGFR antibodies on the x-axis relative to the maximum activation of ACTR-expressing Jurkat cells in combination with the indicated antibody or combination of antibodies on the y-axis. Each circle represents a different cancer tissue, and the size of the circle indicates the relative expression of EGFR on the cells of the cancer. Panel A: Cetuximab with ACTR-expressing Jurkat cells. Panel B: mixture of anti-EGFR antibodies (EGFR A and EGFR B), in combination with ACTR-expressing Jurkat cells.

FIG. 3 is a graph showing activation of ACTR-expressing Jurkat NFAT reporter cells in the presence of CD19 expressing cells and increasing concentrations of the anti-CD19 antibody MOR208. Panel A: CD19-expressing Daudi cells. Panel B: CD19-expressing Raji cells. Luminenscence is a measure of luciferase upregulation, which is regulated by activity of NFAT upon cell activation.

FIG. 4 is a graph showing activation of ACTR-expressing Jurkat NFAT reporter cells in the presence of Daudi cells expressing CD22 and increasing concentrations of the anti-CD22 antibody Epratuzumab. Panel A: Epratuzumab. Panel B: Afucosylated Epratuzumab. Luminenscence is a measure of luciferase upregulation, which is regulated by activity of NFAT upon cell activation.

FIG. 5 is a graph showing activation of ACTR-expressing Jurkat NFAT reporter cells in the presence of Raji cells expressing CD22 and increasing concentrations of the anti-CD22 antibody Epratuzumab. Panel A: Epratuzumab. Panel B: Afucosylated Epratuzumab. Luminenscence is a measure of luciferase upregulation, which is regulated by activity of NFAT upon cell activation.

FIG. 6 is a graph showing that ACTR-expressing primary T cells produce IFNγ in the presence of cells expressing CD19 and increasing concentrations of the anti-CD19 antibody MOR208. Panel A: CD19-expressing RL cells. Panel B: CD19-expressing Raji cells. Panel C: CD19-expressing Namalwa tumor cells.

FIG. 7 is a graph showing that ACTR-expressing primary T cells produce IFNγ in the presence of cells expressing CD22 and increasing concentrations of the anti-CD22 antibody Epratuzumab (afucosylated). Panel A: CD22-expressing RL cells. Panel B: CD22-expressing Raji cells. Panel C: CD22-expressing Namalwa tumor cells.

FIG. 8 is a graph showing enhanced cytotoxicity and cytokine production by ACTR-expressing T cells in the presence of CD19- and CD22-expressing RL cells and the anti-CD19 and anti-CD22 antibodies alone or the combination of both the anti-CD19 and anti-CD22 antibodies. Panel A: Cytotoxicity. Panel B: IFNγ production.

FIG. 9 is a graph demonstrating HER-2 expression level on specific tumor cell lines. The values reported are the Geometric Mean Fluorescent Intensity values of the staining. SK-BR-3 and HCC1954 are considered HER-2 amplified cell lines while ZR-75-1, BT-20 and MCF7 are considered non-amplified cell lines.

FIG. 10 is a graph demonstrating the correlation of NFAT Jurkat activation with HER-2 expression on tumor lines. Activation of ACTR in the presence of a target and target-specific antibody, trastuzumab, was measured by the emission of luminescence with a Jurkat-NFAT reporter line stably expressing ACTR. The level of activation is shown in relative luminescence units (RLU).

FIG. 11 is a graph demonstrating the mediation of ACTR cytotoxicity by target-antibody pairing. The cytotoxicity of HER2+ cell lines mediated by primary ACTR T-cells in combination with trastuzumab was measured at 48 hours as a loss of luminescence compared to target alone controls.

FIG. 12 is a graph demonstrating the pro-inflammatory cytokine response of ACTR T-cells on HER-2 amplified cell lines. Cytokine (IFN-g, IL-2 and TNF-α) production from ACTR T-cells was measured through intracellular cytokine staining at 6 and 18 hours (panels A and B, respectively) in the presence of target, with and without 1 μg/mL of trastuzumab. The total cytokine response from ACTR T-cells was plotted and the subsets of the response are expressed in the graph as parts of the whole.

FIG. 13 is a graph demonstrating the proliferation of primary ACTR T-cells on HER-2 amplified and non-amplified cell lines. The number of ACTR T-cells was determined by flow cytometry and the values are reported as the percent of input ACTR T-cells.

FIG. 14 is a graph demonstrating the cytotoxicity seen in cell lines in which HER-2 is not amplified using trastuzumab and pertuzumab both singly and in combination. The effect of the combination of trastuzumab and pertuzumab on ACTR cytotoxicity was examined in a 48-hour luciferase based cytotoxicity assay at a 2:1 E:T ratio.

FIG. 15 is a graph demonstrating the effect of combining anti-HER-2 antibodies on the pro-inflammatory response in cell lines in which HER-2 is not amplified. The effect of trastuzumab and pertuzumab on the IFN-g, IL-2 and TNF-α cytokine production of ACTR T-cells was explored on two non-amplified HER-2 cell lines (BT-20 and ZR-75-1; panels A and B respectively) at 18 hours. The total cytokine response from ACTR T-cells was plotted and the subsets of the response are expressed as parts of the whole.

FIG. 16 is a graph demonstrating the proliferation seen in cell lines in which HER-2 is not amplified using trastuzumab and pertuzumab both singly and in combination. The effect of the trastuzumab/pertuzumab combination on proliferation of ACTR T-cells was explored in a seven day single stimulation assay. The count of ACTR T-cells was determined by flow cytometry and the values are reported as a percentage of input ACTR T-cells.

FIG. 17 is a graph demonstrating PD-L1 expression on HER-2 positive tumor cell lines. PD-L1 expression on HER-2 positive tumor lines was evaluated by flow cytometry with an anti-human PD-L1 antibody. The reported values are the GMFI of the staining.

FIG. 18 is a series of photographs demonstrating cytotoxicity of ACTR T-cells on a HER-2+/PD-L1+ spheroid. The invasive ability of ACTR T-cells was examined through generation of HER-2+/PD-L1+ HCC1954 spheroids, staining with CellTracker Red, and subsequent imaging to visualize cell death over a three-day period using the Incucyte® system. Panel A: Day 0. Panel B: Day 3 in the presence of HCC1954 and ACTR-T cells. Panel C: Day 3 in the presence of HCC1954, ACTR-T cells, and trastuzumab.

FIG. 19 is a flowchart demonstrating the manner in which the ACTR platform was evaluated with a broad panel of tumor-targeting antibodies against multiple tumor cell lines. The high-throughput triage characterized ACTR activity in a Jurkat-NFAT-luciferase reporter assay and benchmarked activity relative to target binding in a cell-based ELISA.

FIG. 20 is a series of graphs demonstrating the relation between target binding and ACTR activation across exemplary antibodies and targets. Binding to target cells and ACTR activity in a Jurkat-NFAT-luciferase reporter assay were characterized for a panel of tumor-targeting antibodies. Each point represents an individual target cell line. Activity was confirmed with a number of marketed therapeutic antibodies including: trastuzumab, pertuzumab, and cetuximab, including enhanced activity with combinations to the same target. Panel A: anti-HER-2 antibodies (trastuzumab; pertuzumab; and the combination of trastuzumab and pertuzumab) and anti-EGFR antibodies (cetuximab; anti-EGFR A and anti-EGFR B; and anti-EGFR C); Panel B: anti-CS1 (elotuzumab), anti-CD38 (daratumumab), and antibodies against two different Heme tumor targets (A and B); Panel C: antibodies against solid tumor targets (A and B) and Pan tumor targets (A and B).

FIG. 21 is a series of graphs demonstrating the results of targeting multiple epitopes in combination with ACTR. Anti-Her2 antibodies, trastuzumab and pertuzumab, (panel A) and anti-EGFR antibodies, A and B against non-overlapping epitopes (panel B) were used. Relative gene expression levels of Her2 and EGFR for this set of tumor cell lines are depicted in panels C and D, respectively.

FIG. 22 is a series of graphs demonstrating the ability to identify effective and ineffective partners for potential combination with ACTR. Results with elotuzumab and daratumumab using the same panel of multiple myeloma cell lines are shown in panel A. The levels of gene expression for both CS1 and CD38 are shown in panel B.

FIG. 23 is a series of graphs demonstrating the correlation between target binding, ACTR NFAT activity, and the release of IL-2. Results for a hematological tumor target (panel A) and a solid tumor target (panel B) are shown.

FIG. 24 is a series of graphs demonstrating experimental results with an exemplary anti-Pan tumor target antibody identified by the high-throughput triage system. ACTR activity was identified with a pan tumor targeting antibody and further characterized in secondary in vitro assays with primary ACTR T-cells.

FIG. 25 is a graph showing IL-2 production from ACTR T-cells during co-culture with Daudi cells in the presence of antibodies targeting CD19, CD20, or CD22. T-cells expressing a chimeric antigen receptor targeting CD19 (CAR19) were used as a comparator.

FIG. 26 is a graph showing IFNγ production from ACTR T-cells during co-culture with Daudi cells in the presence of antibodies targeting CD19, CD20, or CD22. T-cells expressing CAR19 were used as a comparator.

FIG. 27 is a graph showing ACTR T-cell-mediated tumor cell cytotoxicity during co-culture with Daudi cells in the presence of antibodies targeting CD19, CD20, or CD22. T-cells expressing CAR19 were used as a comparator.

FIG. 28 is a graph showing IFNγ production from ACTR T-cells during co-culture with RL cells in the presence of antibodies targeting CD19 or CD22, or both CD19 and CD22. T-cells expressing CAR19 were used as a comparator.

FIG. 29 is a graph showing RL target cell cytotoxicity during co-culture with ACTR T-cells in the presence of antibodies targeting CD19 or CD22, or both CD19 and CD22. T-cells expressing CAR19 were used as a comparator.

FIG. 30 is an exemplary schematic for a sequential ACTR targeting assay in which ACTR T-cells were co-cultured with Daudi target cells in the presence of anti-CD19, anti-CD20, or anti-CD22 antibodies.

FIG. 31 is a graph showing the cytotoxicity of Daudi cells during a sequential ACTR targeting assay as illustrated in FIG. 28 in which ACTR T-cells were co-cultured with Daudi target cells in the presence of anti-CD19, anti-CD20, or anti-CD22 antibodies. Measurements are shown at day 7 and day 14.

FIG. 32 is a set of graphs comparing the levels of IFNγ and IL-2 (in pg/mL) in Raji cells with wild type CD19 and Raji cells in which CD19 has been deleted. The Raji cells have been incubated with T-cells expressing a CD19-targeting CAR.

FIG. 33 is a graph comparing the levels of IFNγ (in pg/mL) in Raji cells with wild type CD19 and Raji cells in which CD19 has been deleted. The Raji cells have been incubated with ACTR T-cells alone and in combination with anti-CD19, anti-CD20, or anti-CD22 antibodies.

FIG. 34 is a graph comparing the cytotoxicity in Raji cells with wild type CD19 and Raji cells in which CD19 has been deleted. The Raji cells have been incubated with ACTR T-cells alone and in combination with anti-CD19, anti-CD20, or anti-CD22 antibodies.

FIG. 35 is a chart showing the levels of HER2 expression in various tumor cell lines.

FIG. 36 includes charts showing immune responses induced by combined treatment of T cells expressing ACTR construct SEQ ID NO: 67 and trastuzumab in HER2-amplied gastric cancer cell line N87 cells. Panel A: cytotoxicity. Panel B: IL-2 secretion. Panel C: IFN-γ secretion.

FIG. 37 is a chart showing the inhibitory effects of ACTR-T cell/trastuzumab combined treatment on N87 tumors.

FIG. 38 includes charts showing the superior cytotoxicity of ACTR-T cells co-used with the combination of trastuzumab and pertuzumab on HER2-positive cancer cells, as compared with the co-use of ACTR-T cells with either trastuzumab or pertuzumab. Panel A: HT-29 cells. Panel B: MCF-7 cells.

FIG. 39 includes charts showing the induction of IL-2 production by ACTR-T cells co-used with the combination of trastuzumab and pertuzumab on HER2-positive cancer cells, as compared with the co-use of ACTR-T cells with either trastuzumab or pertuzumab. Panel A: MCF-7 cells. Panel B: HT-29 cells. Panel C: NCI-H441 cells. Panel D: SKOV3 cells.

FIG. 40 includes charts showing proliferation of ACTR-T cells in combination with pertuzumab, tratuzumab, or both when incubated with HER-positive cancer cells. Panel A: MCF-7 cells. Panel B: NCI-H441 cells.

### DETAILED DESCRIPTION OF THE INVENTION

Many cancers are characterized by high level of surface expression of cancer antigens, which in some cases is due to gene amplification. For example, HER2 gene amplification occurs in 20-30% of aggressive breast and gastric cancer diagnoses, often signifying poor prognosis. The current standard of care for HER2 amplified cancer is the HER2 targeting antibody Trastuzumab with chemotherapy. In the setting of HER2 amplified breast cancer, the combination of HER2 targeting antibodies Trastuzumab and Pertuzumab with the chemotherapeutic docetaxel is currently the frontline therapy. Despite the success of targeting the HER2 pathway, there are still many patients who are refractory or relapse following HER2 targeting regimens. Cancer cells of such patients often express low levels of HER2. Outside of the setting of HER2 gene amplification, targeting HER2-positive cancers has been largely ineffective. Although, engineered autologous T-cells, including chimeric antigen receptors (CARs) and high affinity T-cell receptors (TCRs), have gained attention due to their potent efficacy, with overall response rates reaching 80% and examples of long lasting remissions, particularly in advanced lymphoma and leukemia, many of the initial clinical attempts to target HER2-amplified breast cancer with CAR-T cell therapy met with either acute toxicities or lack of efficacy. Furthermore, many current cancer treatment regimen have failed due to loss of the targeted antigen, an outcome affecting nearly 65% of patients that failed CAR19 therapy (a CAR-T cell based therapy targeting CD19) (Grupp et al. ASH (December 2015).

The methods described herein are based at least in part on the unexpected findings that the co-use of immune cells expressing the chimeric receptors described herein with at least two anti-cancer antibodies (e.g., HER2 targeting anti-cancer antibody Trastuzumab or Pertuzumab) exhibited potent cytotoxic activity, cytokine response and proliferation on a HER2-amplified tumor cell lines. Furthermore, the effectiveness of immune cells expressing the chimeric receptors in a non-amplified HER2 setting was tested in the presence of a combination of two anti-HER2 antibodies (Trastuzumab and Pertuzumab). This multi-antibody combination increased the cytotoxicity of the immune cells expressing the chimeric receptors on non-amplified HER2 expressing cancer cell lines (e.g., cancer cells having lower expression of HER2 relative to HER2-amplified cancer cells), whereas Trastuzumab or Pertuzumab as single antibody combinations with ACTR had little effect. Similar results were observed in co-use of the immune cells with two anti-cancer antibodies that target EGFR.

The methods described herein also provide for targeting at least two different cancer antigens using at least two anti-cancer antibodies targeting the two different cancer antigens (for example, CD19/CD22 and CD38/CS1).

Accordingly, described herein are co-uses of immune cells expressing chimeric ACTR receptor constructs with at least two anti-cancer antibodies in cancer therapy, particularly in treating cancer expressing low levels of the target cancer antigen.

Chimeric ACTR receptors, comprising an Fc-binding domain, such as the extracellular portion of CD16A, can bind to the Fc portion of antibodies, leading to activation of immune cells such as T cells that express the ACTR and destroying cancer cells targeted by the antibodies (targeting antibodies). Kudo et al., (2014), Cancer Res. 74:93-103, WO2015/058018, and WO2016/040441, the relevant disclosures thereof are incorporated by reference herein. This technology allows for a bridge between the chimeric T cell receptor technology and antibody therapies.

### I. Chimeric Receptors (ACTRs)

Chimeric receptors or antibody-coupled T-cell receptors (ACTRs), as used herein interchangeably, are non-naturally-occurring receptors comprising an Fc binding domain with binding affinity and specificity for an Fc fragment (“Fc binder”) and a cytoplasmic signaling domain. The chimeric receptor may optionally further comprise a co-stimulatory domain, a transmembrane domain, and/or a hinge domain. The chimeric receptors are configured such that, when expressed on a host cell, the Fc binding domain is located extracellularly for binding to an Fc-containing molecule such as membrane-bound Ig and the cytoplasmic signaling domain (as well as the co-stimulatory domain if applicable) is located in the cytoplasm for triggering activation and/or effector signaling. In some embodiments, a chimeric receptor construct as described herein comprises, from N-terminus to C-terminus, the Fc binder, the transmembrane domain, the at least one co-stimulatory signaling domain, and the cytoplasmic signaling domain. In other embodiments, a chimeric receptor construct as described herein comprises, from N-terminus to C-terminus, the Fc binder, the transmembrane domain, the cytoplasmic signaling domains, and the at least one co-stimulatory signaling domain.

Any of the chimeric receptors described herein may further comprise a hinge domain, which may be located at the C-terminus of the Fc binder and the N-terminus of the transmembrane domain. Alternatively or in addition, the chimeric receptor constructs described herein may contain two or more co-stimulatory signaling domains, which may link to each other or be separated by the cytoplasmic signaling domain. The extracellular Fc binder, transmembrane domain, co-stimulatory signaling domain(s), and cytoplasmic signaling domain in a chimeric receptor construct may be linked to each other directly, or via a peptide linker. In some embodiments, the chimeric receptor may not comprise any co-stimulatory domain. Any of the chimeric receptors described herein may be co-expressed in the immune cells with one or more separate polypeptides comprising a co-stimulatory domain or a ligand of a co-stimulation factor, which provide co-stimulatory signals in trans.

A. Fc Binding Domain

The chimeric receptor constructs described herein comprise an Fc binding domain that is an Fc binder, i.e., an Fc fragment of an anti-cancer antibody.

The Fc binder may bind to an Fc portion of any immunoglobulin molecules, for example, naturally occurring immunoglobulin molecules (e.g., IgG, IgA, IgM, or IgE) of a suitable mammal (e.g., human, mouse, rat, goat, sheep, or monkey). Any Fc binding domain known in the art may be used for making the chimeric receptors described herein. Suitable Fc binders may be derived from naturally occurring proteins such as mammalian Fc receptors or certain bacterial proteins (e.g., Protein A or Protein G). Additionally, Fc binders may be synthetic polypeptides, such as single chain antibodies, engineered specifically to bind the Fc portion fragment of an Ig molecule with high affinity and specificity. For example, such an Fc binder can be an antibody or an antigen-binding fragment thereof that specifically binds the Fc portion of an anti-cancer antibody. Examples include, but are not limited to, a single-chain variable fragment (scFv), a domain antibody, or a nanobody. Alternatively, an Fc binder can be a synthetic peptide that specifically binds the Fc portion, such as a Kunitz domain, a small modular immunopharmaceutical (SMIP), an adnectin, an avimer, an affibody, a DARPin, or an anticalin, which may be identified by screening a peptide combinatory library for binding activities to Fc.

Any of the Fc binders described herein may have a suitable binding affinity for an Fc fragment, such as the Fc portion of an anti-cancer antibody. As used herein, “binding affinity” refers to the apparent association constant or K_{A}. The K_{A} is the reciprocal of the dissociation constant, K_{D}. The extracellular ligand-binding domain of an Fc receptor domain of the chimeric receptors described herein may have a binding affinity K_{D} of at least 10⁻⁵, 10⁻⁶, 10⁻⁷, 10⁻⁸, 10⁻⁹, 10⁻¹⁰ M or lower for the Fc portion of an anti-cancer antibody. In some embodiments, the Fc binder has a high binding affinity for an Fc fragment derived from an antibody, isotype of antibodies, or subtype(s) thereof, as compared to the binding affinity of the Fc binder to an Fc fragment derived from another antibody, isotype of antibodies or subtypes thereof. In some embodiments, the extracellular domain of an Fc receptor has specificity for an Fc fragment derived from an antibody, isotype of antibodies, or subtype(s) thereof, as compared to binding of the extracellular domain of an Fc receptor to an Fc fragment derived from another antibody, isotype of antibodies, or subtypes thereof.

The binding affinity or binding specificity for an Fc binding domain or a chimeric receptor comprising an Fc binding domain can be determined by a variety of methods including equilibrium dialysis, equilibrium binding, gel filtration, ELISA, surface plasmon resonance, or spectroscopy.

In some embodiments, the Fc binder is an extracellular domain of a mammalian Fc receptor. As used herein, an “Fc receptor” is a cell surface bound receptor that is expressed on the surface of many immune cells (including B cells, dendritic cells, natural killer (NK) cells, macrophage, neutrophils, mast cells, and eosinophils) and exhibits binding specificity to an Fc domain. Fc receptors are typically comprised of at least two immunoglobulin (Ig)-like domains, which have binding specificity to the Fc portion of an Ig molecule. In some instances, binding of an Fc receptor to an Fc portion of a membrane-bound Ig may trigger cell-mediated cytotoxicity (ADCC) effects, thereby destroying a target cell expressing the membrane-bound Ig.

Fc receptors are classified based on the isotype of the antibody to which it is able to bind. For example, Fc-gamma receptors (FcγR) generally bind to IgG antibodies, such as one or more subtype thereof (i.e., IgG1, IgG2, IgG3, IgG4); Fc-alpha receptors (FcαR) generally bind to IgA antibodies; and Fc-epsilon receptors (FcεR) generally bind to IgE antibodies. In some embodiments, the Fc receptor is an Fc-gamma receptor, an Fc-alpha receptor, or an Fc-epsilon receptor. Examples of Fc-gamma receptors include, without limitation, CD64A, CD64B, CD64C, CD32A, CD32B, CD16A, and CD16B. An example of an Fc-alpha receptor is FcαR1/CD89. Examples of Fc-epsilon receptors include, without limitation, FcεRI and FcεRII/CD23. Table 1 below lists exemplary Fc receptors for use in constructing the chimeric receptors described herein and their binding activity to corresponding Fc domains:

TABLE 1
Exemplary Fc Receptors

| Receptor | Principal antibody | Affinity for |
|---|---|---|
| name | ligand | ligand |
| FcγRI (CD64) | IgG1 and IgG3 | High (Kd~10⁻⁹ M) |
| FcγRIIA (CD32) | IgG | Low (Kd > 10⁻⁷ M) |
| FcγRIIB1 (CD32) | IgG | Low (Kd > 10⁻⁷ M) |
| FcγRIIB2 (CD32) | IgG | Low (Kd > 10⁻⁷ M) |
| FcγRIIIA (CD16a) | IgG | Low (Kd > 10⁻⁶ M) |
| FcγRIIIB (CD16b) | IgG | Low (Kd > 10⁻⁶ M) |
| FcεRI | IgE | High (Kd~10⁻¹⁰ M) |
| FcεRII (CD23) | IgE | Low (Kd > 10⁻⁷ M) |
| FcαRI (CD89) | IgA | Low (Kd > 10⁻⁶ M) |
| Fcα/μR | IgA and IgM | High for IgM, Mid for IgA |
| FcRn | IgG | |

In some embodiments, the Fc receptor used for constructing a chimeric receptor or ACTR as described herein may be a naturally-occurring polymorphism variant (e.g., the CD16 V158 polymorphism variant described herein). Some examples are provided in Table 2 below.

TABLE 2
Exemplary Polymorphisms in Fc Receptors

| Amino Acid | | | | | | | | | | |
|---|---|---|---|---|---|---|---|---|---|---|
| Number | 19 | 48 | 65 | 89 | 105 | 130 | 134 | 141 | 142 | 158 |
| FCR10 | R | S | D | I | D | G | F | Y | T | V |
| P08637 | R | S | D | I | D | G | F | Y | I | F |
| S76824 | R | S | D | I | D | G | F | Y | I | V |
| J04162 | R | N | D | V | D | D | F | H | I | V |
| M31936 | S | S | N | I | D | D | F | H | I | V |
| M24854 | S | S | N | I | E | D | S | H | I | V |
| X07934 | R | S | N | I | D | D | F | H | I | V |
| X14356 (FcγRII) | N | N | N | S | E | S | S | S | I | I |
| M31932 (FcγRI) | S | T | N | R | E | A | F | T | I | G |
| X06948 (FcαεI) | R | S | E | S | Q | S | E | S | I | V |

Selection of the ligand binding domain of an Fc receptor for use in the chimeric receptors described herein will be apparent to one of skill in the art. For example, it may depend on factors such as the binding affinity of the Fc receptor to its ligand, for example, the Fc portion of the anti-cancer antibodies.

The amino acid sequences of the CD16A V158 polymorphism variant are provided below, which can be used for constructing the chimeric receptors described herein. SEQ ID NO: 57 represents the amino acid sequence of the precursor receptor (including the signal sequence, which is underlined), and SEQ ID NO: 58 represents the amino acid sequence of the mature protein.

(CD16A V158, precursor protein)
SEQ ID NO: 57
MWQLLLPTALLLLVSAGMRTEDLPKAVVFLEPQWYRVLEKDSVTLKCQGA
YSPEDNSTQWFHNESLISSQASSYFIDAATVDDSGEYRCQTNLSTLSDPV
QLEVHIGWLLLQAPRWVFKEEDPIHLRCHSWKNTALHKVTYLQNGKGRKY
FHHNSDFYIPKATLKDSGSYFCRGLVGSKNVSSETVNITITQGLAVSTIS
SFFPPGYQVSFCLVMVLLFAVDTGLYFSVKTNIRSSTRDWKDHKFKWRKD
PQDK
(CD16A V158, mature protein)
SEQ ID NO: 58
GMRTEDLPKAVVFLEPQWYRVLEKDSVTLKCQGAYSPEDNSTQWFHNESL
ISSQASSYFIDAATVDDSGEYRCQTNLSTLSDPVQLEVHIGWLLLQAPRW
VFKEEDPIHLRCHSWKNTALHKVTYLQNGKGRKYFHHNSFYIPKATLKDS
GSYFCRGLVGSKNVSSETVNITITQGLAVSTISSFFPPGYQVSFCLVMVL
LFAVDTGLYFSVKTNIRSSTRDWKDHKFKWRKDPQDK

In some embodiments, the Fc binders described herein may be subjected to mutation to achieve a suitable (e.g., enhanced) binding affinity to a wild-type Fc fragment, such as the Fc portion of an anti-cancer antibody. The mutated Fc binding domain of the chimeric receptors described herein may have a binding affinity K_{D} of at least 10⁻⁵, 10⁻⁶10⁻⁷, 10⁻⁸, 10⁻⁹, 10⁻¹⁰M or lower for a wild-type Fc fragment. In some instances, the mutated Fc binder has an enhanced binding affinity for a specific wild-type Fc fragment, isotype, or subtype(s) thereof, for example, anti-cancer antibodies, as compared to the binding affinity of the mutated Fc binder to another Fc fragment, isotype of antibodies or subtypes thereof. In some embodiments, the binding affinity of the mutated Fc binder for the Fc portion of a membrane-bound Ig is about 1.5-fold, 2-fold, 3-fold, 4-fold, 5-fold, 6-fold, 7-fold, 8-fold, 9-fold, 10-fold, 20-fold, 30-fold, 40-fold, 50-fold, or at least 100-fold enhanced as compared to binding affinity of the Fc binder (in the absence of the one or more mutations) for the membrane-bound Ig. Chimeric receptors containing such mutated Fc binders may have an enhanced activity induced by a membrane-bound Ig and thus be more effective in triggering effector activities (e.g., cytotoxicity) against target cells expressing the membrane-bound Ig, for example, cancer cells.

The binding affinity of a chimeric receptor variant comprising a mutated Fc binder or its wild-type counterpart for an Fc domain can be determined by a variety of methods including, without limitation, equilibrium dialysis, equilibrium binding, flow cytometry, gel filtration, ELISA, surface plasmon resonance, or spectroscopy.

In general, the terms “about” and “approximately” mean within an acceptable error range for the particular value as determined by one of ordinary skill in the art, which will depend in part on how the value is measured or determined, i.e., the limitations of the measurement system. For example, in regard to the binding activity of a chimeric receptor variant “about” can mean within an acceptable standard deviation, per the practice in the art. Alternatively, “about” can mean a range of less than ±30%, preferably less than ±20%, more preferably less than ±10%, more preferably less than ±5%, and more preferably still less than ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

In some embodiments, the Fc binding domain of an ACTR described herein comprises an amino acid sequence that is at least 90% (e.g., 91, 92, 93, 94, 95, 96, 97, 98, 99%) identical to the amino acid sequence of the Fc binding domain of a naturally-occurring Fc-gamma receptor, an Fc-alpha receptor, or an Fc-epsilon receptor. The “percent identity” of two amino acid sequences can be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the disclosure. Where gaps exist between two sequences, Gapped BLAST can be utilized as described in Altschul et al., Nucleic Acids Res. 25(17):3389-3402, 1997. When utilizing BLAST and Gapped BLAST programs, one may use the default parameters of the respective programs (e.g., XBLAST and NBLAST).

In some examples, the Fc receptor can be CD16A, CD16B, CD32A, CD32B, CD32C, CD64A, CD64B, CD64C, or a variant thereof as described herein. The extracellular ligand-binding domain of an Fc receptor may comprise up to 10 amino acid residue variations (e.g., 1, 2, 3, 4, 5, or 8) relative to the amino acid sequence of the extracellular ligand-binding domain of a wild-type CD16A, CD16B, CD32A, CD32B, CD32C, CD64A, CD64B, CD64C as described herein. Such Fc domains comprising one or more amino acid variations may be referred to as a variant. Mutation of amino acid residues of the extracellular ligand-binding domain of an Fc receptor may result in an increase in binding affinity for the Fc receptor domain to bind to the Fc portion of a membrane-bound Ig, such as a membrane-bound IgG, on a target cell.

In other embodiments, the Fc binder is derived from a naturally occurring bacterial protein that is capable of binding to the Fc portion of an IgG molecule. An Fc binder for use in constructing a chimeric receptor as described herein can be a full-length protein or a functional fragment thereof. Protein A is a 42 kDa surface protein originally found in the cell wall of the bacterium Staphylococcus aureus. It is composed of five domains that each fold into a three-helix bundle and are able to bind IgG through interactions with the Fc region of most antibodies as well as the Fab region of human VH3 family antibodies. Protein G is an approximately 60-kDa protein expressed in group C and G Streptococcal bacteria that binds to both the Fab and Fc region of mammalian IgGs. While native protein G also binds albumin, recombinant variants have been engineered that eliminate albumin binding.

Such Fc binders for use in constructing the chimeric receptors (ACTRs) described herein may also be created de novo using combinatorial biology or directed evolution methods. Starting with a protein scaffold (e.g., an scFv derived from IgG, a Kunitz domain derived from a Kunitz-type protease inhibitor, an ankyrin repeat, the Z domain from protein A, a lipocalin, a fibronectin type III domain, an SH3 domain from Fyn, or others), amino acid side chains for a set of residues on the surface may be randomly substituted in order to create a large library of variant scaffolds. From large libraries it is possible to isolate rare variants with affinity for a target like the Fc domain by first selecting for binding, followed by amplification by phage, ribosome or cell display. Repeated rounds of selection and amplification can be used to isolate those proteins with the highest affinity for the target.

Alternatively, the extracellular domain of the chimeric receptor variant described herein may be a single chain antibody fragment that binds to an anti-cancer antibody, e.g., binds to its Fc portion.

B. Transmembrane Domain

In some embodiments, the chimeric receptors described herein further comprise a transmembrane domain. The transmembrane domain for use in the chimeric receptors can be in any form known in the art. As used herein, a “transmembrane domain” refers to any protein structure that is thermodynamically stable in a cell membrane, preferably a eukaryotic cell membrane. Transmembrane domains compatible for use in the chimeric receptors used herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times).

Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the chimeric receptor described herein is derived from a Type I single-pass membrane protein. Single-pass membrane proteins include, but are not limited to, CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B. In some embodiments, the transmembrane domain is from a membrane protein selected from the following: CD8α, CD8β, 4-1BB/CD137, CD28, CD34, CD4, FcεRIγ, CD16, OX40/CD134, CD3ζ, CD3ε, CD3γ, CD3δ, TCRα, CD32, CD64, VEGFR2, FAS, and FGFR2B. In some examples, the transmembrane domain is of CD8α. In some examples, the transmembrane domain is of 4-1BB/CD137. In other examples, the transmembrane domain is of CD28 or CD34. In yet other examples, the transmembrane domain is not derived from human CD8α. In some embodiments, the transmembrane domain of the chimeric receptor is a single-pass alpha helix.

Transmembrane domains from multi-pass membrane proteins may also be compatible for use in the chimeric receptors described herein. Multi-pass membrane proteins may comprise a complex (at least 2, 3, 4, 5, 6, 7 or more) alpha helices or a beta sheet structure. Preferably, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side. Either one or multiple helix passes from a multi-pass membrane protein can be used for constructing the chimeric receptor described herein.

Transmembrane domains for use in the chimeric receptors described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 B1 and PCT Publication No. WO 2000/032776 A2, the relevant disclosures of which are incorporated by reference herein.

In some embodiments, the amino acid sequence of the transmembrane domain does not comprise cysteine residues. In some embodiments, the amino acid sequence of the transmembrane domain comprises one cysteine residue. In some embodiments, the amino acid sequence of the transmembrane domain comprises two cysteine residues. In some embodiments, the amino acid sequence of the transmembrane domain comprises more than two cysteine residues (e.g., 3, 4, 5 or more).

The transmembrane domain may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence.

The hydropathy, or hydrophobic or hydrophilic characteristics of a protein or protein segment, can be assessed by any method known in the art, for example the Kyte and Doolittle hydropathy analysis.

C. Co-Stimulatory Signaling Domains

Many immune cells require co-stimulation, in addition to stimulation of an antigen-specific signal, to promote cell proliferation, differentiation and survival, as well as to activate effector functions of the cell. The chimeric receptors described herein may comprise at least one co-stimulatory signaling domain. The term “co-stimulatory signaling domain,” as used herein, refers to at least a portion of a protein that mediates signal transduction within a cell to induce an immune response, such as an effector function. The co-stimulatory signaling domain of the chimeric receptor described herein can be a cytoplasmic signaling domain from a co-stimulatory protein, which transduces a signal and modulates responses mediated by immune cells, such as T cells, NK cells, macrophages, neutrophils, or eosinophils.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the chimeric receptors described herein. The type(s) of co-stimulatory signaling domain is selected based on factors such as the type of the immune cells in which the chimeric receptors would be expressed (e.g., T cells, NK cells, macrophages, neutrophils, or eosinophils) and the desired immune effector function (e.g., ADCC effect). Examples of co-stimulatory signaling domains for use in the chimeric receptors can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thyl, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the co-stimulatory signaling domain is of 4-1BB, CD28, OX40, ICOS, CD27, GITR, HVEM, TIM1, LFA1 (CD11a) or CD2, or any variant thereof. In other embodiments, the co-stimulatory signaling domain is not derived from 4-1BB.

Also within the scope of the present disclosure are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue variations (e.g., 1, 2, 3, 4, 5, or 8) as compared to a wild-type counterpart. Such co-stimulatory signaling domains comprising one or more amino acid variations may be referred to as variants.

Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation. For example, mutation of residues 186 and 187 of the native CD28 amino acid sequence may result in an increase in co-stimulatory activity and induction of immune responses by the co-stimulatory domain of the chimeric receptor. In some embodiments, the mutations are substitution of a lysine at each of positions 186 and 187 with a glycine residue of the CD28 co-stimulatory domain, referred to as a CD²⁸_{LL→GG} variant. Additional mutations that can be made in co-stimulatory signaling domains that may enhance or reduce co-stimulatory activity of the domain will be evident to one of ordinary skill in the art. In some embodiments, the co-stimulatory signaling domain is of 4-1BB, CD28, OX40, or CD²⁸_{LL→-GG} variant. In some embodiments, the chimeric receptors may comprise more than one co-stimulatory signaling domain (e.g., 2, 3 or more). In some embodiments, the chimeric receptor comprises two or more of the same co-stimulatory signaling domains, for example, two copies of the co-stimulatory signaling domain of CD28. In some embodiments, the chimeric receptor comprises two or more co-stimulatory signaling domains from different co-stimulatory proteins, such as any two or more co-stimulatory proteins described herein. Selection of the type(s) of co-stimulatory signaling domains may be based on factors such as the type of host cells to be used with the chimeric receptors (e.g., immune cells such as T cells, NK cells, macrophages, neutrophils, or eosinophils) and the desired immune effector function. In some embodiments, the chimeric receptor comprises two co-stimulatory signaling domains. In some embodiments, the two co-stimulatory signaling domains are CD28 and 4-1BB. In some embodiments, the two co-stimulatory signaling domains are CD²⁸_{LL→-GG} variant and 4-1BB.

Any of the co-stimulatory domains, or a combination thereof, may be part of the chimeric receptors described herein. Chimeric receptors containing a co-stimulatory signaling domain may be co-used (co-introduced into a host cell) with a separate polypeptide, which can be a co-stimulatory factor or comprises the co-stimulatory domain thereof. The separate polypeptide may comprise the same co-stimulatory domain as the chimeric receptor, or a different co-stimulatory domain. Chimeric receptors containing a co-stimulatory signaling domain may also be co-used with a separate polypeptide comprising a ligand of a co-stimulatory factor, which can be the same as or different from that used in the chimeric receptor. See, e.g., Zhao, et al. Cancer Cell (2015) 28:415-428.

Alternatively, chimeric receptors may contain no co-stimulatory domain. Such chimeric receptors may be co-used (co-introduced into a host cell) with one or more separate polypeptides, which can be a co-stimulatory factor or comprises the co-stimulatory domain thereof. Chimeric receptors that do not contain a co-stimulatory signaling domain may also be co-used with a separate polypeptide comprising a ligand of a co-stimulatory factor.

D. Cytoplasmic Signaling Domain

Any cytoplasmic signaling domain can be used to construct the chimeric receptors described herein. In general, a cytoplasmic signaling domain relays a signal, such as interaction of an extracellular ligand-binding domain with its ligand, to stimulate a cellular response, such as inducing an effector function of the cell (e.g., ADCC).

In some embodiments, cytoplasmic signaling domain comprises an immunoreceptor tyrosine-based inhibition motif (ITIM). In some embodiments, the cytoplasmic signaling domain comprises an immunoreceptor tyrosine-based activation motif (ITAM). An “ITIM” and an “ITAM,” as used herein, are conserved protein motifs that are generally present in the tail portion of signaling molecules expressed in many immune cells.

The ITIM motif comprises the amino acid sequence S/I/V/LxYxxl/V/L (SEQ ID NO: 68). Upon stimulation of an ITIM, the motif becomes phosphorylated and reduces activation of molecules involved in cell signaling, thereby transducing an inhibitory signal.

In some examples, the cytoplasmic domain comprising an ITIM is of a Killer-cell immunoglobulin-like receptor (KIR).

The ITAM motif may comprise two repeats of the amino acid sequence YxxL/I separated by 6-8 amino acids, wherein each x is independently any amino acid, producing the conserved motif YxxL/Ix₍₆₋₈₎YxxL/I (SEQ ID NO: 69). ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. In some examples, the cytoplasmic signaling domain comprising an ITAM is of CD3ζ or FcεR1γ. In other examples, the ITAM-containing cytoplasmic signaling domain is not derived from human CD3ζ. In yet other examples, the ITAM-containing cytoplasmic signaling domain is not derived from an Fc receptor, when the extracellular ligand-binding domain of the same chimeric receptor construct is derived from CD16A.

E. Hinge Domain

In some embodiments, the chimeric receptors described herein further comprise a hinge domain that is located between the extracellular ligand-binding domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular ligand-binding domain of an Fc receptor relative to the transmembrane domain of the chimeric receptor can be used.

The hinge domain may contain about 10-200 amino acids, e.g., 15-150 amino acids, 20-100 amino acids, or 30-60 amino acids. In some embodiments, the hinge domain may be of about 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, or 200 amino acids in length.

In some embodiments, the hinge domain is a hinge domain of a naturally occurring protein. Hinge domains of any protein known in the art to comprise a hinge domain are compatible for use in the chimeric receptors described herein. In some embodiments, the hinge domain is at least a portion of a hinge domain of a naturally occurring protein and confers flexibility to the chimeric receptor. In some embodiments, the hinge domain is of CD8α. In some embodiments, the hinge domain is a portion of the hinge domain of CD8α, e.g., a fragment containing at least 15 (e.g., 20, 25, 30, 35, or 40) consecutive amino acids of the hinge domain of CD8α.

Hinge domains of antibodies, such as an IgG, IgA, IgM, IgE, or IgD antibody, are also compatible for use in the chimeric receptors described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the chimeric receptors described herein. In some embodiments, the hinge domain between the C-terminus of the extracellular ligand-binding domain of an Fc receptor and the N-terminus of the transmembrane domain is a peptide linker, such as a (GlyₓSer)ₙ linker, wherein x and n, independently can be an integer between 3 and 12, including 3, 4, 5, 6, 7, 8, 9, 10, 11, 12, or more. In some embodiments, the hinge domain is (Gly₄Ser)ₙ (SEQ ID NO: 59), wherein n can be an integer between 3 and 60, including 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more. In some embodiments, the hinge domain is (Gly₄Ser)₃ (SEQ ID NO: 60). In some embodiments, the hinge domain is (Gly₄Ser)₆ (SEQ ID NO: 61). In some embodiments, the hinge domain is (Gly₄Ser)₉ (SEQ ID NO: 61). In some embodiments, the hinge domain is (Gly₄Ser)₁₂ (SEQ ID NO: 62). In some embodiments, the hinge domain is (Gly₄Ser)₁₅ (SEQ ID NO: 63). In some embodiments, the hinge domain is (Gly₄Ser)₃₀ (SEQ ID NO: 64). In some embodiments, the hinge domain is (Gly₄Ser)₄₅ (SEQ ID NO: 65). In some embodiments, the hinge domain is (Gly₄Ser)₆₀ (SEQ ID NO: 66).

In other embodiments, the hinge domain is an extended recombinant polypeptide (XTEN), which is an unstructured polypeptide consisting of hydrophilic residues of varying lengths (e.g., 10-200 amino acid residues, 20-150 amino acid residues, 30-100 amino acid residues, or 40-80 amino acid residues). Amino acid sequences of XTEN peptides will be evident to one of skill in the art and can be found, for example, in U.S. Pat. No. 8,673,860, which is herein incorporated by reference. In some embodiments, the hinge domain is an XTEN peptide and comprises 60 amino acids. In some embodiments, the hinge domain is an XTEN peptide and comprises 30 amino acids. In some embodiments, the hinge domain is an XTEN peptide and comprises 45 amino acids. In some embodiments, the hinge domain is an XTEN peptide and comprises 15 amino acids.

F. Signal Peptide

In some embodiments, the chimeric receptor also comprises a signal peptide (also known as a signal sequence) at the N-terminus of the polypeptide. In general, signal sequences are peptide sequences that target a polypeptide to the desired site in a cell. In some embodiments, the signal sequence targets the chimeric receptor to the secretory pathway of the cell and will allow for integration and anchoring of the chimeric receptor into the lipid bilayer. Signal sequences including signal sequences of naturally occurring proteins or synthetic, non-naturally occurring signal sequences, that are compatible for use in the chimeric receptors described herein will be evident to one of skill in the art. In some embodiments, the signal sequence from CD8α. In some embodiments, the signal sequence is from CD28. In other embodiments, the signal sequence is from the murine kappa chain. In yet other embodiments, the signal sequence is from CD16. An example signal sequence is provided by amino acid residues 1-16 of SEQ ID NO: 1.

Table 3 provides exemplary chimeric receptors described herein. This exemplary constructs have, from N-terminus to C-terminus in order, the signal sequence, the Fc binder (e.g., an extracellular domain of an Fc receptor), the hinge domain, and the transmembrane, while the positions of the co-stimulatory domain and the cytoplasmic signaling domain can be switched.

TABLE 3
Exemplary chimeric receptors

| Exemplary | | | | | | |
|---|---|---|---|---|---|---|
| AA | | | | | | |
| Sequence | | Extracellular | | | Co- | Cytoplasmic |
| (SEQ ID | Signal | domain of | Hinge | Transmembrane | stimulatory | Signaling |
| NO) | Sequence | Fc receptor | domain | domain | domain | domain |
| 1 | CD8α | CD16A- | CD8α | CD8α | 4-1BB | CD3ζ |
| | | V158 | | | (CD137) | |
| 2 | CD8α | CD16A- | CD8α | 4-1BB | 4-1BB | CD3ζ |
| | | V158 | | (CD137) | (CD137) | |
| 3 | CD8α | CD16A- | CD8α | CD28 | 4-1BB | CD3ζ |
| | | V158 | | | (CD137) | |
| 4 | CD8α | CD16A- | CD8α | CD34 | 4-1BB | CD3ζ |
| | | V158 | | | (CD137) | |
| 5 | CD8α | CD16A- | CD8α | Designed | 4-1BB | CD3ζ |
| | | V158 | | hydrophobic | (CD137) | |
| | | | | TM domain | | |
| 6 | CD8α | CD32A | CD8α | CD8α | 4-1BB | CD3ζ |
| | | | | | (CD137) | |
| 7 | CD8α | CD16A- | CD8α | CD8α | CD28 | CD3ζ |
| | | V158 | | | | |
| 8 | CD8α | CD16A- | CD8α | CD8α | OX40 | CD3ζ |
| | | V158 | | | (CD134) | |
| 9 | CD8α | CD16A- | CD8α | CD8α | CD28 + | CD3ζ |
| | | V158 | | | 4-1BB | |
| 10 | CD8α | CD16A- | None | CD8α | 4-1BB | CD3ζ |
| | | V158 | | | (CD137) | |
| 11 | CD8α | CD16A- | XTEN | CD8α | 4-1BB | CD3ζ |
| | | V158 | | | (CD137) | |
| 12 | CD8α | CD16A- | CD8α | CD8α | CD28 LL | CD3ζ |
| | | V158 | | | to GG | |
| | | | | | mutant | |
| 13 | CD8α | CD16A- | CD8α | CD8α | CD28 LL | CD3ζ |
| | | V158 | | | to GG | |
| | | | | | mutant + | |
| | | | | | 4-1BB | |
| 14 | CD8α | CD16A- | CD8α | CD4 | 4-1BB | CD3ζ |
| | | V158 | | | (CD137) | |
| 15 | CD8α | CD16A- | CD8α | CD4 | CD28 LL | CD3ζ |
| | | V158 | | | to GG | |
| | | | | | mutant + | |
| | | | | | 4-1BB | |
| 16 | CD8α | CD16A- | CD8α | FcεR1γ | 4-1BB | CD3ζ |
| | | V158 | | | (CD137) | |
| 17 | CD8α | CD16A- | CD8α | Designed | 4-1BB | CD3ζ |
| | | V158 | | hydrophobic | (CD137) | |
| | | | | TM domain, | | |
| | | | | predicted | | |
| | | | | dimerization | | |
| 18 | CD8α | CD16A- | CD8α | CD8β | 4-1BB | CD3ζ |
| | | V158 | | | (CD137) | |
| 19 | CD8α | CD16A- | CD8α | C16α | 4-1BB | CD3ζ |
| | | V158 | | | (CD137) | |
| 20 | CD8α | CD16A- | CD8α | OX40 (CD134) | 4-1BB | CD3ζ |
| | | V158 | | | (CD137) | |
| 21 | CD8α | CD16A- | CD8α | CD3ζ | 4-1BB | CD3ζ |
| | | V158 | | | (CD137) | |
| 22 | CD8α | CD16A- | CD8α | CD3ε | 4-1BB | CD3ζ |
| | | V158 | | | (CD137) | |
| 23 | CD8α | CD16A- | CD8α | CD3γ | 4-1BB | CD3ζ |
| | | V158 | | | (CD137) | |
| 24 | CD8α | CD16A- | CD8α | CD3δ | 4-1BB | CD3ζ |
| | | V158 | | | (CD137) | |
| 25 | CD8α | CD16A- | CD8α | TCR-α | 4-1BB | CD3ζ |
| | | V158 | | | (CD137) | |
| 26 | CD8α | CD16A- | CD8α | CD32 | 4-1BB | CD3ζ |
| | | V158 | | | (CD137) | |
| 27 | CD8α | CD16A- | CD8α | CD64 | 4-1BB | CD3ζ |
| | | V158 | | | (CD137) | |
| 28 | CD8α | CD16A- | CD8α | VEGFR2 | 4-1BB | CD3ζ |
| | | V158 | | | (CD137) | |
| 29 | CD8α | CD16A- | CD8α | FAS | 4-1BB | CD3ζ |
| | | V158 | | | (CD137) | |
| 30 | CD8α | CD16A- | CD8α | FGFR2B | 4-1BB | CD3ζ |
| | | V158 | | | (CD137) | |
| 31 | CD8α | CD16A- | CD8α | CD8α | 4-1BB | CD3ζ |
| | | F158 | | | (CD137) | |
| 32 | CD8α | CD64A | CD8α | CD8α | 4-1BB | CD3ζ |
| | | | | | (CD137) | |
| 33 | CD8α | CD16A- | IgG1 (hinge- | CD8α | 4-1BB | CD3ζ |
| | | V158 | CH2-CH3) | | (CD137) | |
| 34 | CD8α | CD16A- | IgG1 (hinge- | CD8α | 4-1BB | CD3ζ |
| | | V158 | CH3) | | (CD137) | |
| 35 | CD8α | CD16A- | IgG1 (hinge) | CD8α | 4-1BB | CD3ζ |
| | | V158 | | | (CD137) | |
| 36 | CD8α | CD16A- | CD8-alpha | CD8α | 4-1BB | CD3ζ |
| | | V158 | fragment 1 | | (CD137) | |
| | | | (30 amino | | | |
| | | | acids) | | | |
| 37 | CD8α | CD16A- | CD8-alpha | CD8α | 4-1BB | CD3ζ |
| | | V158 | fragment 2 | | (CD137) | |
| | | | (15 amino | | | |
| | | | acids) | | | |
| 38 | CD8α | CD16A- | (G1y4Ser)x3 | CD8α | 4-1BB | CD3ζ |
| | | V158 | (60 amino | | (CD137) | |
| | | | acids) | | | |
| 39 | CD8α | CD16A- | (G1y4Ser)x6 | CD8α | 4-1BB | CD3ζ |
| | | V158 | (45 amino | | (CD137) | |
| | | | acids) | | | |
| 40 | CD8α | CD16A- | (G1y4Ser)x9 | CD8α | 4-1BB | CD3ζ |
| | | V158 | (30 amino | | (CD137) | |
| | | | acids) | | | |
| 41 | CD8α | CD16A- | (G1y4Ser)x12 | CD8α | 4-1BB | CD3ζ |
| | | V158 | (15 amino | | (CD137) | |
| | | | acids) | | | |
| 42 | CD8α | CD16A- | XTEN (60 | CD8α | 4-1BB | CD3ζ |
| | | V158 | amino acids) | | (CD137) | |
| 43 | CD8α | CD16A- | XTEN (30 | CD8α | 4-1BB | CD3ζ |
| | | V158 | amino acids) | | (CD137) | |
| 44 | CD8α | CD16A- | XTEN (15 | CD8α | 4-1BB | CD3ζ |
| | | V158 | amino acids) | | (CD137) | |
| 45 | CD28 | CD16A- | CD8α | CD8α | 4-1BB | CD3ζ |
| | | V158 | | | (CD137) | |
| 46 | Murine | CD16A- | CD8α | CD8α | 4-1BB | CD3ζ |
| | kappa | V158 | | | (CD137) | |
| | chain | | | | | |
| 47 | CD16 | CD16A- | CD8α | CD8α | 4-1BB | CD3ζ |
| | | V158 | | | (CD137) | |
| 48 | CD8α | CD16A- | CD8α | CD8α | ICOS | CD3ζ |
| | | V158 | | | | |
| 49 | CD8α | CD16A- | CD8α | CD8α | CD27 | CD3ζ |
| | | V158 | | | | |
| 50 | CD8α | CD16A- | CD8α | CD8α | GITR | CD3ζ |
| | | V158 | | | | |
| 51 | CD8α | CD16A- | CD8α | CD8α | HVEM | CD3ζ |
| | | V158 | | | | |
| 52 | CD8α | CD16A- | CD8α | CD8α | TIM1 | CD3ζ |
| | | V158 | | | | |
| 53 | CD8α | CD16A- | CD8α | CD8α | LFA1 | CD3ζ |
| | | V158 | | | (CD1 la) | |
| 54 | CD8α | CD16A- | CD8α | CD8α | CD2 | CD3ζ |
| | | V158 | | | | |
| 55 | CD8α | CD16A- | CD8α | FcεR1γ | 4-1BB | FcεR1γ |
| | | V158 | | | (CD137) | |
| 56 | CD8α | CD16A- | CD8α | CD8α | 4-1BB | FcεR1γ |
| | | V158 | | | (CD137) | |
| 67 | | CD16A- | CD28 | CD28 | CD28 | CD3ζ |
| | | V158 | | | | |

Amino acid sequences of the example chimeric receptors are provided below (signal peptide italicized; which can be replaced with another suitable signal peptide).

SEQ ID NO: 1:
MALPVTALLLPLALLLHAARPGMRTEDLPKAVVFLEPQWYRVLEKDSVTLKCQGAYSPEDNS
TQWFHNESLISSQASSYFIDAATVDDSGEYRCQTNLSTLSDPVQLEVHIGWLLLQAPRWVFK
EEDPIHLRCHSWKNTALHKVTYLQNGKGRKYFHHNSDFYIPKATLKDSGSYFCRGLVGSKNV
SSETVNITITQGLAVSTISSFFPPGYQTTTPAPRPPTPAPTIASQPLSLRPEACRPAAGGAV
HTRGLDFACDIYIWAPLAGTCGVLLLSLVITLYCKRGRKKLLYIFKQPFMRPVQTTQEEDGC
SCRFPEEEEGGCELRVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTATKDTYDALHMQALP
PR
SEQ ID NO: 2:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIISFFLALTSTALLFLLFFLTLRFSVVKRGKRGRKKLLYIFK
QPFMRPVQTTQEEDGCSCRFPEEEEGGCELRVKFSRSADAPAYQQGQNQLYNELNLGRRE
EYDVLDKRRGRDPEMGGKPRRKNPQEGLYNELQKDKMAEAYSEIGMKGERRRGKGHDGLY
QGLSTATKDTYDALHMQALPPR
SEQ ID NO: 3:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDFWVLVVVGGVLACYSLLVTVAFIIFWVRSKKRGRKKLLYIFK
QPFMRPVQTTQEEDGCSCRFPEEEEGGCELRVKFSRSADAPAYQQGQNQLYNELNLGRRE
EYDVLDKRRGRDPEMGGKPRRKNPQEGLYNELQKDKMAEAYSEIGMKGERRRGKGHDGLY
QGLSTATKDTYDALHMQALPPR
SEQ ID NO: 4:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDLIALVTSGALLAVLGITGYFLMNRKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 5:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDLLAALLALLAALLALLAALLARSKKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 6:
MALPVTALLLPLALLLHAARPQAAAPPKAVLKLEPPWINVLQEDSVTLTCQGARSPESDS
IQWFHNGNLIPTHTQPSYRFKANNNDSGEYTCQTGQTSLSDPVHLTVLSEWLVLQTPHLE
FQEGETIMLRCHSWKDKPLVKVTFFQNGKSQKFSHLDPTFSIPQANHSHSGDYHCTGNIG
YTLFSSKPVTITVQVPSMGSSSPMGTTTPAPRPPTPAPTIASQPLSLRPEACRPAAGGAV
HTRGLDFACDIYIWAPLAGTCGVLLLSLVITLYCKRGRKKLLYIFKQPFMRPVQTTQEED
GCSCRFPEEEEGGCELRVKFSRSADAPAYQQGQNQLYNELNLGRREEYDVLDKRRGRDPE
MGGKPRRKNPQEGLYNELQKDKMAEAYSEIGMKGERRRGKGHDGLYQGLSTATKDTYDAL
HMQALPPR
SEQ ID NO: 7:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCRSKRSRLLHSDYMNMTPR
RPGPTRKHYQPYAPPRDFAAYRSRVKFSRSADAPAYQQGQNQLYNELNLGRREEYDVLDK
RRGRDPEMGGKPRRKNPQEGLYNELQKDKMAEAYSEIGMKGERRRGKGHDGLYQGLSTAT
KDTYDALHMQALPPR
SEQ ID NO: 8:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCALYLLRRDQRLPPDAHKP
PGGGSFRTPIQEEQADAHSTLAKIRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 9:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCRSKRSRLLHSDYMNMTPR
RPGPTRKHYQPYAPPRDFAAYRSKRGRKKLLYIFKQPFMRPVQTTQEEDGCSCRFPEEEE
GGCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
SEQ ID NO: 10:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQIYIWAPLAGTCGVLLLSLVITLYCKRG
RKKLLYIFKQPFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAYSEIGMKGERR
RGKGHDGLYQGLSTATKDTYDALHMQALPPR
SEQ ID NO: 11:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQGGSPAGSPTSTEEGTSESATPESGPGT
STEPSEGSAPGSPAGSPTIYIWAPLAGTCGVLLLSLVITLYCKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 12:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCRSKRSRGGHSDYMNMTPR
RPGPTRKHYQPYAPPRDFAAYRSRVKFSRSADAPAYQQGQNQLYNELNLGRREEYDVLDK
RRGRDPEMGGKPRRKNPQEGLYNELQKDKMAEAYSEIGMKGERRRGKGHDGLYQGLSTAT
KDTYDALHMQALPPR
SEQ ID NO: 13:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCRSKRSRGGHSDYMNMTPR
RPGPTRKHYQPYAPPRDFAAYRSKRGRKKLLYIFKQPFMRPVQTTQEEDGCSCRFPEEEE
GGCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
SEQ ID NO: 14:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDMALIVLGGVAGLLLFIGLGIFFCVRKRGRKKLLYIFKQPFMR
PVQTTQEEDGCSCRFPEEEEGGCELRVKFSRSADAPAYQQGQNQLYNELNLGRREEYDVL
DKRRGRDPEMGGKPRRKNPQEGLYNELQKDKMAEAYSEIGMKGERRRGKGHDGLYQGLST
ATKDTYDALHMQALPPR
SEQ ID NO: 15:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDMALIVLGGVAGLLLFIGLGIFFCVRRSKRSRGGHSDYMNMTP
RRPGPTRKHYQPYAPPRDFAAYRSRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 16:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDLCYILDAILFLYGIVLTLLYCRLKKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 17:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDLLLILLGVLAGVLATLAALLARSKKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 18:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDITLGLLVAGVLVLLVSLGVAIHLCKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 19:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDVSFCLVMVLLFAVDTGLYFSVKTNKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 20:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDVAAILGLGLVLGLLGPLAILLALYKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 21:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDLCYLLDGILFIYGVILTALFLRVKKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 22:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDVMSVATIVIVDICITGGLLLLVYYWSKNRKRGRKKLLYIFKQ
PFMRPVQTTQEEDGCSCRFPEEEEGGCELRVKFSRSADAPAYQQGQNQLYNELNLGRREE
YDVLDKRRGRDPEMGGKPRRKNPQEGLYNELQKDKMAEAYSEIGMKGERRRGKGHDGLYQ
GLSTATKDTYDALHMQALPPR
SEQ ID NO: 23:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDGFLFAEIVSIFVLAVGVYFIAGQDKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 24:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDGIIVTDVIATLLLALGVFCFAGHETKRGRKKLLYIFKQPFMR
PVQTTQEEDGCSCRFPEEEEGGCELRVKFSRSADAPAYQQGQNQLYNELNLGRREEYDVL
DKRRGRDPEMGGKPRRKNPQEGLYNELQKDKMAEAYSEIGMKGERRRGKGHDGLYQGLSI
ATKDTYDALHMQALPPR
SEQ ID NO: 25:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDVIGFRILLLKVAGFNLLMTLRLWKRGRKKLLYIFKQPFMRPV
QTTQEEDGCSCRFPEEEEGGCELRVKFSRSADAPAYQQGQNQLYNELNLGRREEYDVLDK
RRGRDPEMGGKPRRKNPQEGLYNELQKDKMAEAYSEIGMKGERRRGKGHDGLYQGLSTAT
KDTYDALHMQALPPR
SEQ ID NO: 26:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIIVAVVIATAVAAIVAAVVALIYCRKKRGRKKLLYIFKQPFM
RPVQTTQEEDGCSCRFPEEEEGGCELRVKFSRSADAPAYQQGQNQLYNELNLGRREEYDV
LDKRRGRDPEMGGKPRRKNPQEGLYNELQKDKMAEAYSEIGMKGERRRGKGHDGLYQGLS
TATKDTYDALHMQALPPR
SEQ ID NO: 27:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDVLFYLAVGIMFLVNTVLWVTIRKEKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 28:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIIILVGTAVIAMFFWLLLVIILRTKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 29:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDLGWLCLLLLPIPLIVWVKRKKRGRKKLLYIFKQPFMRPVQTT
QEEDGCSCRFPEEEEGGCELRVKFSRSADAPAYQQGQNQLYNELNLGRREEYDVLDKRRG
RDPEMGGKPRRKNPQEGLYNELQKDKMAEAYSEIGMKGERRRGKGHDGLYQGLSTATKDT
YDALHMQALPPR
SEQ ID NO: 30:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIAIYCIGVFLIACMVVTVILCRMKKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 31:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
FGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 32:
MALPVTALLLPLALLLHAARPQVDTTKAVITLQPPWVSVFQEETVTLHCEVLHLPGSSSI
QWFLNGTATQTSTPSYRITSASVNDSGEYRCQRGLSGRSDPIQLEIHRGWLLLQVSSRVF
TEGEPLALRCHAWKDKLVYNVLYYRNGKAFKFFHWNSNLTILKTNISHNGTYHCSGMGKH
RYTSAGISVTVKELFPAPVLNASVTSPLLEGNLVTLSCETKLLLQRPGLQLYFSFYMGSK
TLRGRNTSSEYQILTARREDSGLYWCEAATEDGNVLKRSPELELQVLGLQLPTPVWFHIY
IWAPLAGTCGVLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
SEQ ID NO: 33:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQEPKSCDKTHTCPPCPAPELLGGPSVFL
FPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQQGNV
FSCSVMHEALHNHYTQKSLSLSPGKIYIWAPLAGTCGVLLLSLVITLYCKRGRKKLLYIF
KQPFMRPVQTTQEEDGCSCRFPEEEEGGCELRVKFSRSADAPAYQQGQNQLYNELNLGRR
EEYDVLDKRRGRDPEMGGKPRRKNPQEGLYNELQKDKMAEAYSEIGMKGERRRGKGHDGL
YQGLSTATKDTYDALHMQALPPR
SEQ ID NO: 34:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQEPKSCDKTHTCPGQPREPQVYTLPPSR
DELTKNQVSLTCLVKGFYPSDIAVEWESNGQPENNYKTTPPVLDSDGSFFLYSKLTVDKS
RWQQGNVFSCSVMHEALHNHYTQKSLSLSPGKIYIWAPLAGTCGVLLLSLVITLYCKRGR
KKLLYIFKQPFMRPVQTTQEEDGCSCRFPEEEEGGCELRVKFSRSADAPAYQQGQNQLYN
ELNLGRREEYDVLDKRRGRDPEMGGKPRRKNPQEGLYNELQKDKMAEAYSEIGMKGERRR
GKGHDGLYQGLSTATKDTYDALHMQALPPR
SEQ ID NO: 35:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQEPKSCDKTHTCPIYIWAPLAGTCGVLL
LSLVITLYCKRGRKKLLYIFKQPFMRPVQTTQEEDGCSCRFPEEEEGGCELRVKFSRSAD
APAYQQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR
SEQ ID NO: 36:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAF
ACDIYIWAPLAGTCGVLLLSLVITLYCKRGRKKLLYIFKQPFMRPVQTTQEEDGCSCRFP
EEEEGGCELRVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYDALHMQALPP
R
SEQ ID NO: 37:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPFACDIYIWAPLAGTCG
VLLLSLVITLYCKRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
SEQ ID NO: 38:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQGGGGSGGGGSGGGGSIYIWAPLAGTCG
VLLLSLVITLYCKRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
SEQ ID NO: 39:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQGGGGSGGGGSGGGGSGGGGSGGGGSGG
GGSIYIWAPLAGTCGVLLLSLVITLYCKRGRKKLLYIFKQPFMRPVQTTQEEDGCSCRFP
EEEEGGCELRVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYDALHMQALPP
R
SEQ ID NO: 40:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQGGGGSGGGGSGGGGSGGGGSGGGGSGG
GGSGGGGSGGGGSGGGGSIYIWAPLAGTCGVLLLSLVITLYCKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 41:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQGGGGSGGGGSGGGGSGGGGSGGGGSGG
GGSGGGGSGGGGSGGGGSGGGGSGGGGSGGGGSIYIWAPLAGTCGVLLLSLVITLYCKRG
RKKLLYIFKQPFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAYSEIGMKGERR
RGKGHDGLYQGLSTATKDTYDALHMQALPPR
SEQ ID NO: 42:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQGGSPAGSPTSTEEGTSESATPESGPGT
STEPSEGSAPGSPAGSPTSTEEGTSTEPSEGSAIYIWAPLAGTCGVLLLSLVITLYCKRG
RKKLLYIFKQPFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAYSEIGMKGERR
RGKGHDGLYQGLSTATKDTYDALHMQALPPR
SEQ ID NO: 43:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQGGSPAGSPTSTEEGTSESATPESGPGT
STEIYIWAPLAGTCGVLLLSLVITLYCKRGRKKLLYIFKQPFMRPVQTTQEEDGCSCRFP
EEEEGGCELRVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYDALHMQALPP
R
SEQ ID NO: 44:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQGGSPAGSPTSTEEGTIYIWAPLAGTCG
VLLLSLVITLYCKRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
SEQ ID NO: 45:
MLRLLLALNLFPSIQVTGGMRTEDLPKAVVFLEPQWYRVLEKDSVILKCQGAYSPEDNST
QWFHNESLISSQASSYFIDAATVDDSGEYRCQTNLSTLSDPVQLEVHIGWLLLQAPRWVF
KEEDPIHLRCHSWKNTALHKVTYLQNGKGRKYFHHNSDFYIPKATLKDSGSYFCRGLVGS
KNVSSETVNITITQGLAVSTISSFFPPGYQTTTPAPRPPTPAPTIASQPLSLRPEACRPA
AGGAVHTRGLDFACDIYIWAPLAGTCGVLLLSLVITLYCKRGRKKLLYIFKQPFMRPVQT
TQEEDGCSCRFPEEEEGGCELRVKFSRSADAPAYQQGQNQLYNELNLGRREEYDVLDKRR
GRDPEMGGKPRRKNPQEGLYNELQKDKMAEAYSEIGMKGERRRGKGHDGLYQGLSTATKD
TYDALHMQALPPR
SEQ ID NO: 46:
METDTLLLWVLLLWVPGSTGDGMRTEDLPKAVVFLEPQWYRVLEKDSVILKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 47:
MWQLLLPTALLLLVSAGMRTEDLPKAVVFLEPQWYRVLEKDSVILKCQGAYSPEDNSTQW
FHNESLISSQASSYFIDAATVDDSGEYRCQTNLSTLSDPVQLEVHIGWLLLQAPRWVFKE
EDPIHLRCHSWKNTALHKVTYLQNGKGRKYFHHNSDFYIPKATLKDSGSYFCRGLVGSKN
VSSETVNITITQGLAVSTISSFFPPGYQTTTPAPRPPTPAPTIASQPLSLRPEACRPAAG
GAVHTRGLDFACDIYIWAPLAGTCGVLLLSLVITLYCKRGRKKLLYIFKQPFMRPVQTTQ
EEDGCSCRFPEEEEGGCELRVKFSRSADAPAYQQGQNQLYNELNLGRREEYDVLDKRRGR
DPEMGGKPRRKNPQEGLYNELQKDKMAEAYSEIGMKGERRRGKGHDGLYQGLSTATKDTY
DALHMQALPPR
SEQ ID NO: 48:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCCWLTKKKYSSSVHDPNGE
YMFMRAVNTAKKSRLTDVTLRVKFSRSADAPAYQQGQNQLYNELNLGRREEYDVLDKRRG
RDPEMGGKPRRKNPQEGLYNELQKDKMAEAYSEIGMKGERRRGKGHDGLYQGLSTATKDT
YDALHMQALPPR
SEQ ID NO: 49:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCQRRKYRSNKGESPVEPAE
PCRYSCPREEEGSTIPIQEDYRKPEPACSPRVKFSRSADAPAYQQGQNQLYNELNLGRRE
EYDVLDKRRGRDPEMGGKPRRKNPQEGLYNELQKDKMAEAYSEIGMKGERRRGKGHDGLY
QGLSTATKDTYDALHMQALPPR
SEQ ID NO: 50:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCQLGLHIWQLRSQCMWPRE
TQLLLEVPPSTEDARSCQFPEEERGERSAEEKGRLGDLWVRVKFSRSADAPAYQQGQNQL
YNELNLGRREEYDVLDKRRGRDPEMGGKPRRKNPQEGLYNELQKDKMAEAYSEIGMKGER
RRGKGHDGLYQGLSTATKDTYDALHMQALPPR
SEQ ID NO: 51:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCCVKRRKPRGDVVKVIVSV
QRKRQEAEGEATVIEALQAPPDVTTVAVEETIPSFTGRSPNHRVKFSRSADAPAYQQGQN
QLYNELNLGRREEYDVLDKRRGRDPEMGGKPRRKNPQEGLYNELQKDKMAEAYSEIGMKG
ERRRGKGHDGLYQGLSTATKDTYDALHMQALPPR
SEQ ID NO: 52:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCKKYFFKKEVQQLSVSFSS
LQIKALQNAVEKEVQAEDNIYIENSLYATDRVKFSRSADAPAYQQGQNQLYNELNLGRRE
EYDVLDKRRGRDPEMGGKPRRKNPQEGLYNELQKDKMAEAYSEIGMKGERRRGKGHDGLY
QGLSTATKDTYDALHMQALPPR
SEQ ID NO: 53:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCYKVGFFKRNLKEKMEAGR
GVPNGIPAEDSEQLASGQEAGDPGCLKPLHEKDSESGGGKDRVKFSRSADAPAYQQGQNQ
LYNELNLGRREEYDVLDKRRGRDPEMGGKPRRKNPQEGLYNELQKDKMAEAYSEIGMKGE
RRRGKGHDGLYQGLSTATKDTYDALHMQALPPR
SEQ ID NO: 54:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCKRKKQRSRRNDEELETRA
HRVATEERGRKPHQIPASTPQNPATSQHPPPPPGHRSQAPSHRPPPPGHRVQHQPQKRPP
APSGTQVHQQKGPPLPRPRVQPKPPHGAAENSLSPSSNRVKFSRSADAPAYQQGQNQLYN
ELNLGRREEYDVLDKRRGRDPEMGGKPRRKNPQEGLYNELQKDKMAEAYSEIGMKGERRR
GKGHDGLYQGLSTATKDTYDALHMQALPPR
SEQ ID NO: 55:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDPQLCYILDAILFLYGIVLTLLYCRLKIQVRKAAITSYEKSDG
VYTGLSTRNQETYETLKHEKPPQKRGRKKLLYIFKQPFMRPVQTTQEEDGCSCRFPEEEE
GGCEL
SEQ ID NO: 56:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCKRGRKKLLYIFKQPFMRP
VQTTQEEDGCSCRFPEEEEGGCELRLKIQVRKAAITSYEKSDGVYTGLSTRNQETYETLK
HEKPPQ
SEQ ID NO: 67:
MALPVTALLLPLALLLHAARPGMRTEDLPKAVVFLEPQWYRVLEKDSVTLKCQGAYSPEDNS
TQWFHNESLISSQASSYFIDAATVDDSGEYRCQTNLSTLSDPVQLEVHIGWLLLQAPRWVFK
EEDPIHLRCHSWKNTALHKVTYLQNGKGRKYFHHNSDFYIPKATLKDSGSYFCRGLVGSKNV
SSETVNITITQGLAVSTISSFFPPGYQIEVMYPPPYLDNEKSNGTIIHVKGKHLCPSPLFPG
PSKPFWVLVVVGGVLACYSLLVTVAFIIFWVRSKRSRLLHSDYMNMTPRRPGPTRKHYQPYA
PPRDFAAYRSRVKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDALHMQALPPR

In particular examples, the chimeric receptors (ACTR constructs) described herein may comprise an extracellular domain of CD16A (either the V158 or F158 variant of, e.g., human) and a cytoplasmic signaling domain of CD3ζ (e.g., human). Such a chimeric receptor may comprise a co-stimulatory domain of 4-1BB or CD28. In some examples, the chimeric receptor may further comprise a hinge domain of CD8a or CD28 (e.g., human).

Exemplary chimeric receptors described herein may comprise an extracellular domain of human CD16A (either the V158 or F158 variant), a hinge domain of human CD8α, a co-stimulatory domain of human 4-1BB, and a cytoplasmic signaling domain of human CD3ζ. Alternatively, exemplary chimeric receptors described herein may comprise an extracellular domain of human CD16A (either the V158 or F158 variant), a hinge domain of human CD28, a co-stimulatory domain of human CD28, and a cytoplasmic signaling domain of human CD3ζ.

Any of the chimeric receptors described herein can be prepared by a routine method, such as recombinant technology. Methods for preparing the chimeric receptors herein involve generation of a nucleic acid that encodes a polypeptide comprising each of the domains of the chimeric receptors, including the Fc binding, the transmembrane domain, at least one co-stimulatory signaling domain, and the cytoplasmic signaling domain. In some embodiments, the nucleic acid also encodes a hinge domain between the extracellular ligand-binding domain of an Fc receptor and the transmembrane domain. The nucleic acid encoding the chimeric receptor may also encode a signal sequence. In some embodiments, the nucleic acid sequence encodes any one of the exemplary chimeric receptors provided by SEQ ID NO: 1-56 and 67.

Sequences of each of the components of the chimeric receptors may be obtained via routine technology, e.g., PCR amplification from any one of a variety of sources known in the art. In some embodiments, sequences of one or more of the components of the chimeric receptors are obtained from a human cell. Alternatively, the sequences of one or more components of the chimeric receptors can be synthesized. Sequences of each of the components (e.g., domains) can be joined directly or indirectly (e.g., using a nucleic acid sequence encoding a peptide linker) to form a nucleic acid sequence encoding the chimeric receptor, using methods such as PCR amplification or ligation. Mutation of one or more residues, for example one or more residues within the extracellular ligand-binding domain that are involved in interaction of the Fc receptor with an antibody, may be made in the nucleic acid sequence encoding said domain prior to or after joining the sequences of each of the components. Alternatively, the nucleic acid encoding the chimeric receptor may be synthesized. In some embodiments, the nucleic acid is DNA. In other embodiments, the nucleic acid is RNA.

### II. Host Cells Expressing Chimeric Receptors

Host cells expressing the chimeric receptors described herein provide a specific population of cells that can recognize target cells, e.g., cancer cells that are bound by at least two anti-cancer antibodies. Engagement of the extracellular ligand-binding domain of a chimeric receptor construct expressed on such host cells (e.g., immune cells) with the Fc portion of an anti-cancer antibody transmits an activation signal to the cytoplasmic signaling domain, and optionally the co-stimulatory signaling domain(s), of the chimeric receptor construct, which in turn activates cell proliferation and/or effector functions of the host cell, such as ADCC effects triggered by the host cells. The combination of co-stimulatory signaling domain(s) and the cytoplasmic signaling domain may allow for robust activation of multiple signaling pathways within the cell that expresses the chimeric receptor.

In some embodiments, the host cells are immune cells, such as T cells, NK cells, macrophages, neutrophils, eosinophils, or any combination thereof. In some embodiments, the immune cells are T cells. In some embodiments, the immune cells are NK cells. In other embodiments, the immune cells can be established cell lines, for example, NK-92 cells. In some embodiments, the host cells are cells that can develop and/or differentiate into immune cells, for example progenitor cells.

The population of immune cells can be obtained from any source, such as peripheral blood mononuclear cells (PBMCs), bone marrow, tissues such as spleen, lymph node, thymus, or tumor tissue. A source suitable for obtaining the type of host cells desired would be evident to one of skill in the art. In some embodiments, the population of immune cells is derived from PBMCs. In some embodiments, the population of immune cells is derived from a human cancer patient, such as from the bone marrow or from a tumor in a human cancer patient. In some embodiments, the population of immune cells is derived from a healthy donor. The type of host cells desired (e.g., immune cells such as T cells, NK cells, macrophages, neutrophils, eosinophils, or any combination thereof) may be expanded within the population of cells obtained by co-incubating the cells with stimulatory molecules, for example, anti-CD3 and anti-CD28 antibodies may be used for expansion of T cells.

To construct the immune cells that express any of the chimeric receptor constructs described herein, expression vectors for stable or transient expression of the chimeric receptor construct may be constructed via conventional methods as described herein and introduced into immune host cells. For example, nucleic acids encoding the chimeric receptors may be cloned into a suitable expression vector, such as a viral vector in operable linkage to a suitable promoter. The nucleic acids and the vector may be contacted, under suitable conditions, with a restriction enzyme to create complementary ends on each molecule that can pair with each other and be joined with a ligase. Alternatively, synthetic nucleic acid linkers can be ligated to the termini of the nucleic acid encoding the chimeric receptors. The synthetic linkers may contain nucleic acid sequences that correspond to a particular restriction site in the vector. The selection of expression vectors/plasmids/viral vectors would depend on the type of host cells for expression of the chimeric receptors, but should be suitable for integration and replication in eukaryotic cells.

A variety of promoters can be used for expression of the chimeric receptors described herein, including, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters for expression of the chimeric receptors include any constitutively active promoter in an immune cell. Alternatively, any regulatable promoter may be used, such that its expression can be modulated within an immune cell.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′- and 3′-untranslated regions for mRNA stability and translation efficiency from highly-expressed genes like α-globin or β-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. See section VI below. Suitable vectors and methods for producing vectors containing transgenes are well known and available in the art. Examples of the preparation of vectors for expression of chimeric receptors can be found, for example, in US2014/0106449, herein incorporated by reference in its entirety.

In some embodiments, the chimeric receptor construct or the nucleic acid encoding said chimeric receptor is a DNA molecule. In some embodiments, the chimeric receptor construct or the nucleic acid encoding said chimeric receptor is a transposon. In some embodiments, the chimeric receptor construct or the nucleic acid encoding said chimeric receptor is a plasmid. In some embodiments, chimeric receptor construct or the nucleic acid encoding said chimeric receptor is a DNA plasmid may be electroporated to immune cells (see, e.g., Till, et al. Blood (2012) 119(17): 3940-3950). In some embodiments, the nucleic acid encoding the chimeric receptor is an RNA molecule, which may be electroporated to immune cells.

Any of the vectors comprising a nucleic acid sequence that encodes a chimeric receptor construct described herein is also within the scope of the present disclosure. Such a vector may be delivered into host cells such as host immune cells by a suitable method. Methods of delivering vectors to immune cells are well known in the art and may include DNA, RNA, or transposon electroporation, transfection reagents such as liposomes or nanoparticles to delivery DNA, RNA, or transposons; delivery of DNA, RNA, or transposons or protein by mechanical deformation (see, e.g., Sharei et al. Proc. Natl. Acad. Sci. USA (2013) 110(6): 2082-2087); or viral transduction. In some embodiments, the vectors for expression of the chimeric receptors are delivered to host cells by viral transduction. Exemplary viral methods for delivery include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors, and adeno-associated virus (AAV) vectors (see, e.g., PCT Publication Nos. WO 94/12649, WO 93/03769; WO 93/19191; WO 94/28938; WO 95/11984 and WO 95/00655). In some embodiments, the vectors for expression of the chimeric receptors are retroviruses. In some embodiments, the vectors for expression of the chimeric receptors are lentiviruses. In some embodiments, the vectors for expression of the chimeric receptors are gamma-retroviruses. In some embodiments, the vectors for expression of the chimeric receptors are adeno-associated viruses (AAVs).

In examples in which the vectors encoding chimeric receptors are introduced to the host cells using a viral vector, viral particles that are capable of infecting the immune cells and carry the vector may be produced by any method known in the art and can be found, for example in PCT Application No. WO 1991/002805A2, WO 1998/009271 A1, and U.S. Pat. No. 6,194,191. The viral particles are harvested from the cell culture supernatant and may be isolated and/or purified prior to contacting the viral particles with the immune cells.

Following introduction into the host cells a vector encoding any of the chimeric receptors provided herein, the cells are cultured under conditions that allow for expression of the chimeric receptor. In examples in which the nucleic acid encoding the chimeric receptor is regulated by a regulatable promoter, the host cells are cultured in conditions wherein the regulatable promoter is activated. In some embodiments, the promoter is an inducible promoter and the immune cells are cultured in the presence of the inducing molecule or in conditions in which the inducing molecule is produced. Determining whether the chimeric receptor is expressed will be evident to one of skill in the art and may be assessed by any known method, for example, detection of the chimeric receptor-encoding mRNA by quantitative reverse transcriptase PCR (qRT-PCR) or detection of the chimeric receptor protein by methods including Western blotting, fluorescence microscopy, and flow cytometry. Alternatively, expression of the chimeric receptor may take place in vivo after the immune cells are administered to a subject.

Alternatively, expression of a chimeric receptor construct in any of the immune cells disclosed herein can be achieved by introducing RNA molecules encoding the chimeric receptor constructs. Such RNA molecules can be prepared by in vitro transcription or by chemical synthesis. The RNA molecules can then be introduced into suitable host cells such as immune cells (e.g., T cells, NK cells, macrophages, neutrophils, eosinophils, or any combination thereof) by, e.g., electroporation, transfection reagents, viral transduction or mechanical deformation of cells. For example, RNA molecules can be synthesized and introduced into host immune cells following the methods described in Rabinovich et al., Human Gene Therapy, 17:1027-1035 and WO WO2013/040557.

Methods for preparing host cells expressing any of the chimeric receptors described herein may also comprise activating the host cells ex vivo. Activating a host cell means stimulating a host cell into an activate state in which the cell may be able to perform effector functions (e.g., ADCC). Methods of activating a host cell will depend on the type of host cell used for expression of the chimeric receptors. For example, T cells may be activated ex vivo in the presence of one or more molecule such as an anti-CD3 antibody, an anti-CD28 antibody, IL-2, IL-17, IL-15, or phytohemoagglutinin. In other examples, NK cells may be activated ex vivo in the presence of one or molecules such as a 4-1BB ligand, an anti-4-1BB antibody, IL-15, an anti-IL-15 receptor antibody, IL-2, IL12, IL-21, and K562 cells. In some embodiments, the host cells expressing any of the chimeric receptors described herein are activated ex vivo prior to administration to a subject. Determining whether a host cell is activated will be evident to one of skill in the art and may include assessing expression of one or more cell surface markers associated with cell activation, expression or secretion of cytokines, and cell morphology.

The methods of preparing host cells expressing any of the chimeric receptors described herein may comprise expanding the host cells ex vivo. Expanding host cells may involve any method that results in an increase in the number of cells expressing chimeric receptors, for example, allowing the host cells to proliferate or stimulating the host cells to proliferate. Methods for stimulating expansion of host cells will depend on the type of host cell used for expression of the chimeric receptors and will be evident to one of skill in the art. In some embodiments, the host cells expressing any of the chimeric receptors described herein are expanded ex vivo prior to administration to a subject.

In some embodiments, the host cells expressing the chimeric receptors are expanded and activated ex vivo prior to administration of the cells to the subject. In some embodiments, the ex vivo expansion and/or activation polarizes the host cells to a desired phenotype, for example, T cells or NK cells.

### III. Application of Host Cells Expressing Chimeric Receptor in Cancer Immunotherapy

Host cells (e.g., immune cells) expressing the chimeric receptors (the encoding nucleic acids or vectors comprising such) described herein are useful for inducing cytotoxicity (e.g., v a ADCC) against target cells such as cancer cells that are bound by at least two anti-cancer antibodies, which can be of any isotype, either in vitro or in vivo (e.g., in a subject in need of the treatment).

As used herein, the term “subject” refers to any mammal, such as a human, monkey, mouse, rabbit, or domestic mammal. In some embodiments, the subject is a human. In some embodiments, the subject is a human cancer patient. In some embodiments, the subject is a human subject at risk of developing cancer. A subject, such as a human patient as described herein suitable for the treatment described herein may be identified via routine practice. For example, a biological sample suspected of containing cancer cells can be obtained from a candidate subject. An assay for detecting cancer cells, as those known in the art or described herein, can be performed to determine whether the biological sample contains cancer cells and if so, whether the cancer cells express one or more cancer antigens.

The selection of a suitable chimeric receptor would depend on the type of anti-cancer antibodies administered to the subject, which would be within the knowledge of a skilled person in the art. For example, if one or more of the anti-cancer antibodies is an IgG antibody (or Fc-containing fragment thereof), a chimeric receptor comprising an Fc-binder to the Fc of IgG (e.g., the extracellular domain of an FcR that binds IgG, for example, CD16, CD32, or CD64, can be used). In some embodiments, the at least two anti-cancer antibodies are of different isotypes, and chimeric receptors comprising an Fc-binder to the Fc of said isotypes are used. See Table 1 above for binding specificities of various Fc receptors to specific isotypes of Ig molecules. The immune cells expressing the chimeric receptor as described herein can act indirectly on target cells (e.g., cancer cells) via the interaction between the chimeric receptor and the Fc domain of the antibodies bound to cancer antigen(s) on the surface of the target cell, thereby triggering cytotoxicity against the target cell.

The immune cells expressing the chimeric receptors described herein may be autologous to the subject, i.e., the immune cells are obtained from the subject in need of the treatment, genetically engineered for expression of the chimeric receptor constructs, and then administered to the same subject. Administration of autologous cells to a subject may result in reduced rejection of the host cells as compared to administration of non-autologous cells. Alternatively, the host cells are allogeneic cells, i.e., the cells are obtained from a first subject, genetically engineered for expression of the chimeric receptor construct, and administered to a second subject that is different from the first subject but of the same species. For example, allogeneic immune cells may be derived from a human donor and administered to a human recipient who is different from the donor.

The immune cells, such as T lymphocyte, may be an allogeneic T lymphocyte. Such immune cells may be from donors with partially matched HLA subtypes or with epigenetic profiles with reduced chance for inducing graft-versus-host disease. Alternatively, virally-selected immune cells may be used. In some examples, the allogeneic immune cells such as T cells can be engineered to reduce the graft versus host effects. For example, the expression of the endogenous T cell receptor can be inhibited or eliminated. Alternatively or in addition, expression of one or more components of the Major Histocompatibility Complex (MHC) Class I and/or Class II complex (e.g., β-2-microglobulin) can be reduced or eliminated. In other examples, a natural killer cell inhibitory receptor can be expressed on the T lymphocyte.

In some embodiments, the immune cells are administered to a subject in an amount effective in triggering cytotoxicity (e.g., ADCC activity) to inhibit target cells (e.g., cancer cells) expressing a surface Ig by least 20%, e.g., 30%, 40%, 50%, 80%, 90%, 95%, or above. In some embodiments, the amount of the immune cells may be sufficient to block the growth or replication of cancer cells, reduce tumor volumes, and/or reduce at least one symptom associated with the target disease. The terms “treat,” “treatment,” and the like mean to relieve or alleviate at least one symptom associated with a target disease such as a target cancer, or to slow or reverse the progression of such a disease. Within the meaning of the present disclosure, the term “treat” also denotes to arrest, delay the onset (i.e., the period prior to clinical manifestation of a disease) and/or reduce the risk of developing or worsening a disease. For example, in connection with cancer the term “treat” may mean eliminate or reduce a patient's tumor burden, or prevent, delay or inhibit metastasis, etc.

The efficacy of an immunotherapy using the immune cells expressing any of the chimeric receptors described herein may be assessed by any method known in the art and would be evident to a skilled medical professional. For example, the efficacy of the immunotherapy may be assessed by survival of the subject, assessment of one or more symptoms associated with the disease or disorder, tumor or cancer burden in the subject or tissue or sample thereof, or infectious agent burden in the subject or tissue or sample thereof.

Any of the immune cells expressing chimeric receptors described herein may be prepared or formulated in a pharmaceutically acceptable carrier or excipient as a pharmaceutical composition, which is also within the scope of the present disclosure.

The phrase “pharmaceutically acceptable,” as used in connection with compositions and/or cells of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

To practice the method disclosed herein, an effective amount of the immune cells expressing chimeric receptors or compositions thereof can be administered to a subject (e.g., a human patient) in need of the treatment via a suitable route, such as intravenous administration. Any of the immune cells expressing chimeric receptors or compositions thereof may be administered to a subject in an effective amount. As used herein, an “effective amount” or a “therapeutically effective amount” refers to the amount of the respective agent (e.g., the host cells expressing chimeric receptors, or compositions thereof) that upon administration confers a therapeutic effect on the subject. Determination of whether an amount of the cells or compositions described herein achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject.

In some embodiments, the subject is a human. In some embodiments, the subject is a human cancer patient. In some embodiments, the subject is a human patient suffering from a cancer derived from tissue of the breast, haematopoietic origin, colon/large intestine, liver, lung, ovary, salivary gland, skin, stomach, or upper aerodigestive tract. In some embodiments, the cancer cells are of epithelial origin, such as breast carcinoma cells, colon carcinoma cells, liver carcinoma cells and lung carcinoma cells. In some embodiments, the cancer cells are of B cell origin, such a B-lineage acute lymphoblastic leukemia cells, B-cell chronic lymphocytic leukemia cells, B-cell non-Hodgkin's lymphoma cells, Hairy cell leukemia cells, and multiple myeloma cells. In yet other embodiments, the human cancer patient is suffering from a solid tumor.

The immune cells expressing chimeric receptors are administered with at least two (e.g., 2, 3, 4, 5, or more) anti-cancer antibodies. In some embodiments, the immune cells expressing chimeric receptors are administered with two anti-cancer antibodies. In some embodiments, one or more of the anti-cancer antibody is a polyclonal antibody. As would be evident to one of skill in the art, polyclonal antibodies are a population of antibodies that bind to different epitopes of an antigen. In some embodiments, the immune cells expressing chimeric receptors are administered with a polyclonal anti-cancer antibody. In some embodiments, one or more of the anti-cancer antibody is a monoclonal antibody.

An anti-cancer antibody can be an antibody that specifically binds a cancer antigen, which can be a surface protein (e.g., receptor) differentially expressed on cancer cells as compared with non-cancerous cells. In some examples, such a cancer antigen expresses on cancer cells at a higher level as relative to non-cancerous cells. In other examples, a cancer antigen may be specifically expressed on cancer cells, which means that the expression of such an antigen on non-cancerous cells are not detectable or at a significantly low level. In some instances, the cancer antigen is a variant of a surface protein expressed on non-cancerous cells. Suitable cancer antigens are known in the art and/or described. See, e.g., Van der Bruggen et al. Cancer Immunity (2013). Non-limiting examples of cancer antigens include BCMA, CA15-3, CA19-9, CA27-29, CA125, calcitonin, calretinin, CD19, CD20, CD22, CD24, CD29, CD30, CD33, CD34, CD38, CD44, CD45, CD49f, CD52, CD117, CD133, CgA, chromogranin, CS1, cytokeratin, desmin, eEF2, EGFR, EMA, EPCA-2, EpCAm, ESA, GFAP, GCDFP-15, HER2/ErbB2, HMB-45, LIV-1, Ly-6A/E, MAGE-a9, NSE, NY ESO-1/CAG-3, P63, PAI-1, PD-L1, PLAP, PSA, S100, Sca1, SPAS-1, TMEFF2, TTF-1, Tumor M2-PK, uPA, VEGF, vimentin, and WT-1.

In some embodiments, the cancer antigen is HER2 or EGFR. In some embodiments, the cancer antigens are CD19 and CD22. In some embodiments, the cancer antigens are CD20 and CD22. In some embodiments, the cancer antigens are CD20 and CD19. In some embodiments, the cancer antigens are CD38 and CS1. In some embodiments, the cancer antigens are BCMA and CS1. In some embodiments, the cancer antigens are BCMA and CD38.

Suitable anti-cancer antibodies are known in the art and/or described herein. See, e.g., WO2015/058018, and WO2016/040441, the relevant disclosures of which are incorporated by reference herein. Examples include, but are not limited to, Ocaratuzumab (AME/Lilly), Margetuximab (Macrogenics), MOR00208/MOR208 (MOR/Xencor), Ecromeximab (Kyowa/Life Sci. Pharma.), PF-04605412 (Pfizer/Xencor), hu14.18K322A, EPR4106 (ab108403, Abcam), HuMax-CD38 (Genmab), ABT-806, Adalimumab, Ado-Trastuzumab emtansine, Alemtuzumab, Basiliximab, Bevacizumab, Belimumab, Brentuximab, Canakinumab, Cetuximab, Daclizumab, Daratumumab, Denosumab, Dinoutuzimab, Eculizumab, Efalizumab, Elotuzumab, Epratuzumab, Gemtuzumab, Golimumab, Infliximab, Ipilimumab, Labetuzumab, Natalizumab, Necitumumab, Nimotuzumab, Obinutuzumab, Ofatumumab, Omalizumab, Palivizumab, Panitumumab, Pertuzumab, Ramucirumab, Ritutimab, Sym004 (combination of anti-EGFR antibodies 992 and 1024; Symphogen), anti-EGFR antibody 992 (Symphogen), anti-EGFR antibody 1024 (Symphogen), Tocilizumab, Trastuzumab, Ustekinumab, Vedolizumab, Zalutumumab, mogamulizumab (Koywa/BioWa), Obinutuzumab (Glycart/Roche), Ublituximab (LFB), Imgatuzumab (Glycart/Roche), BIW-8962 (Kyowa/BioWa), MDX-1401 (Medarex/BMS/BioWa), KB004 (KaloBios), ARGX-110 (arGEN-X), ARGX-111 (arGEN-X), SCT200 (Sinocelltech), GC1118 (Green Cross), and MM-151 (Merrimack Pharmaceuticals).

Selection of the anti-cancer antibodies will depend on various factors including the type of cancer or cancer cells targeted by the immunotherapy and the level of expression of the cancer antigen(s) targeted by the anti-cancer antibodies. In some embodiments, the at least two anti-cancer antibodies for use in the methods described herein bind to at least two different cancer antigens. For example, one anti-cancer antibody can bind to one cancer antigen and another anti-cancer antibody can bind to a different cancer antigen. In some embodiments, the anti-cancer antigens are CD19 and CD22 and at least one of the antibodies target (bind to) CD19 and the other antibody targets (bind to) CD22. In some embodiments, the anti-CD19 antibody is MOR208. In some embodiments, the anti-CD22 antibody is Epratuzumab, which can be in an afucosylated form. In some embodiments, the cancer antigens are CD20 and CD19, and at least one of the antibodies targets (binds to) CD20 and the other antibody targets (binds to) CD19. In some embodiments, the cancer antigens are CD20 and CD22, and at least one of the antibodies targets (binds to) CD20 and another antibody targets (binds to) CD22. In some embodiments, the anti-cancer antigens are CD38 and CS1. In some embodiments, the anti-cancer antigens are CD38 and CS1 and at least one of the antibodies target (bind to) CD38 and the other antibody targets (bind to) CS1. In some embodiments, the anti-CD38 antibody is Daratumumab. In some embodiments, the anti-CS1 antibody is Elotuzumab. In some embodiments, the anti-cancer antigens are BCMA and CD38 and at least one antibody targets (binds) BCMA and the other antibody targets (binds to) CD38. In some embodiments, the anti-cancer antigens are BCMA and CS1, and at least one of the antibodies targets (binds) BCMA and the other antibody targets (binds to) CS1.

In some embodiments, the at least two anti-cancer antibodies bind to different epitopes of the same cancer antigen (e.g., are non-competing antibodies).

In some embodiments, the cancer antigen is HER2 and the at least two antibodies bind to HER2 (e.g., bind to different epitopes of HER2). In some embodiments, the anti-HER2 antibodies bind domain IV of HER2 (e.g., Trastuzumab) and domain II of HER2 and blocks HER2 dimerization (e.g., Pertuzumab). The combination of such two anti-HER2 antibodies, together with ACTR-T cells, may be used in treating human patients having HER2 non-amplified cancer cells. Such a human patient can be identified via a routine method known in the art or disclosed herein. See, e.g., Examples below.

In some embodiments, the cancer antigen is EGFR and the at least two antibodies target (bind to) EGFR. In some embodiments, the cancer antigen is EGFR and the anti-EGFR antibodies are 992 and 1024, which comprise Sym004. In some embodiments, the cancer antigen is EGFR and the anti-EGFR antibodies are cetuximab, panitumumab, MM-151, SCT200, GC1118, Necitumumab, Nimotuzumab, Zalutumumab, anti-EGFR antibody 992, anti-EGFR antibody 1024, or a combination thereof.

As described herein the immune cells are co-used with at least two anti-cancer antibodies to target cancer cells, for example, cancer cells having lower expression of a cancer antigen (e.g., HER2, EGFR, or others described herein) or cancer cells that are resistant to other therapies. The expression level of a target cancer antigen can be measured by routine methods, e.g., an immune assay or flow cytometry. Such an assay may involve the use of an antibody specific to the target cancer antigen. See also Examples below. It would be within the knowledge of those skilled in the art to determine whether the expression level of a target cancer antigen being high or low on target cancer cells, which would depend on the type of cancer antigen. In some cases, a target antigen may be overexpressed in cancer cells (e.g., a high level of expression), which may due to amplification or the gene coding for the antigen or dysregulation of expression of the antigen. In other cases, a target antigen may be expressed on cancer cells in a level substantially similar to or even lower than that on normal, non-cancer cells (e.g., a low level of expression).

In some embodiments, a subject who is suitable for the treatment described herein can be identified via measuring via a routine method the expression level of the target cancer antigen (to which at least one of the antibodies used in the treatment binds) in a biological sample obtained from the subject that is suspected of containing cancer cells.

In some embodiments, the host cells expressing such chimeric receptors are administered in the presence of or in combination with the anti-cancer antibodies. In such a therapy, the anti-cancer antibodies may bind to a cancer antigen or more than one cancer antigen and the chimeric receptor. Binding or interaction of the anti-cancer antibodies with a cell may indicate that the cell is a cancer cell. The immune cells and anti-cancer antibodies may be administered simultaneously or sequentially. In some embodiments, the immune cells and the anti-cancer antibodies are administered at least 4 hours apart, e.g., at least 12 hours, at least 1 day, at least 3 days, at least one week, at least two weeks, or at least one month apart.

In some embodiments, the at least two anti-cancer antibodies are administered simultaneously or sequentially. In some embodiments, the two antibodies are used sequentially to reduce the risk of cancer relapse. For example, the anti-cancer antibodies are administered at least 4 hours apart, e.g., at least 12 hours, at least 1 day, at least 3 days, at least one week, at least two weeks, or at least one month apart. In some embodiments, the treatment with the second antibody is at least 1 month, at least 3 month, at least 6 months, or at least 1 year after the treatment with the first antibody. In some embodiments, a second anti-cancer antibody is administered after detecting antigen loss of the cancer antigen targeted by the first anti-cancer antibody or relapse of the cancer.

In some embodiments, the subject (e.g., a human patient) has previously undergone treatment with one of the anti-cancer antibodies used in the methods described herein.

### IV. Combination Treatments

The compositions and methods described in the present disclosure may be utilized in conjunction with other types of therapy for treating a disease or disorder associated with the cancer. In some embodiments, the compositions and methods described herein are utilized in conjunction with a therapy for cancer, such as chemotherapy, surgery, radiation, gene therapy, and so forth. Such therapies can be administered simultaneously or sequentially (in any order) with the immunotherapy described herein.

When co-administered with an additional therapeutic agent, suitable therapeutically effective dosages for each agent may be lowered due to the additive action or synergy.

The immunotherapies described herein can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 41BB, OX40, etc.).

Non-limiting examples of other therapeutic agents useful for combination with the immunotherapies described herein include without limitation: (i) anti-angiogenic agents (e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000)); (ii) a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof; and (iii) chemotherapeutic compounds such as, e.g., pyrimidine analogs (5-fluorouracil, floxuridine, capecitabine, gemcitabine and cytarabine), purine analogs, folate antagonists and related inhibitors (mercaptopurine, thioguanine, pentostatin and 2-chlorodeoxyadenosine (cladribine)); (iii) antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); (iv) antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); (v) antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); (vi) antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); (vii) anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (Trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); (viii) growth factor signal transduction kinase inhibitors; mitochondrial dysfunction inducers and caspase activators; (ix) chromatin disruptors, and (x) immunomodulatory drugs (IMiDs), such as thalidomide and derivatives thereof (lenalidomide, pomalidomide, and apremilast).

In some embodiments, radiation or radiation and chemotherapy are used in combination with the antibody-based immunotherapies described herein.

For examples of additional useful agents see also Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

### VI. Kits for Therapeutic Use

The present disclosure also provides kits for use of the chimeric receptors in immunotherapy of cancer with any of the immune cells disclosed herein, which express a chimeric receptor (ACTR), and at least two anti-cancer antibodies. Such kits may include one or more containers comprising a pharmaceutical composition that comprises any nucleic acid for expressing a chimeric receptor or host cells (e.g., immune cells such as those described herein), and a pharmaceutically acceptable carrier. The kit may further comprise a second pharmaceutical composition, such as those described herein, (e.g., an Fc-containing therapeutic agent such as an anti-cancer antibody or combination of at least two anti-cancer antibodies as described herein) and a pharmaceutically acceptable carrier.

In some embodiments, the kit can comprise instructions for use in any of the methods described herein. The included instructions can comprise a description of administration of the immune cell-containing pharmaceutical compositions and/or the anti-cancer antibody containing pharmaceutical compositions to a subject to achieve the intended activity in a subject. The kit may further comprise a description of selecting a subject suitable for treatment based on identifying whether the subject carries disease cells (e.g., cancer cells). In some embodiments, the instructions comprise a description of administering the pharmaceutical composition to a subject who is in need of the treatment.

The instructions relating to the use of the chimeric receptors and the pharmaceutical compositions described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a chimeric receptor variant as described herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### VII. In Vitro Assays

The present disclosure also provides an in vitro method for evaluating the activity of an immune cell that expresses a chimeric receptor. The in vitro method described herein may comprise: (a) incubating the immune cell expressing the chimeric receptor with a first target cell in the presence of a first agent that comprises an Fc domain, wherein the chimeric receptor comprises an Fc binding domain, and a cytoplasmic signaling domain; and wherein the first agent binds a surface receptor of the first target cell; (b) measuring cytotoxicity of the first target cell induced by the immune cell in the presence of the first agent; (c) separating the immune cell from the first target cell and the first agent; (d) incubating the immune cell obtained in step (c) with a second target cell in the presence of a second agent that comprises an Fc domain, wherein the second agent binds a surface receptor of the second target cell; and (e) measuring cytotoxicity of the second target cell induced by the immune cell in the presence of the second agent.

The in vitro method may be performed with any of the immune cells disclosed herein which express a chimeric receptor (ACTR). The chimeric receptor may further comprise a transmembrane domain, a co-stimulatory signaling domain, a hinge domain, or a combination thereof.

The first and second agents comprising an Fc domain may be any agents comprising an Fc domain known in the art. For example, the agent may be an antibody, or antigen-binding fragment (e.g., a F(ab) or a F(ab)₂). In one embodiment, the agent may be an anti-cancer antibody or antigen-binding fragment thereof. For example, the agent (i.e., the first agent, the second agent, or both the first agent and the second agent) may be antibodies or antigen binding fragments thereof. As a specific example, the first and/or second agent may be an antibody specific to a cell surface receptor. In certain embodiments, the first agent and the second agent may be specific to the same cell surface receptor. In other embodiments, the first agent and the second agent may be specific to different cell surface receptors.

The in vitro method described herein may measure cytotoxicity (i.e., the number or percentage of cells that are killed when co-incubated with the immune cell). For example, the cytotoxicity of the first target cell and/or the second target cell may be more than 5%, more than 10%, more than 15%, more than 20%, more than 25%, more than 30%, more than 35%, more than 40%, more than 45%, more than 50%, more than 55%, more than 60%, more than 65%, more than 70%, more than 75%, more than 80%, more than 85%, more than 90%, more than 95%, more than 96%, more than 97%, more than 98%, or more than 99% of the cultured cells. This measure of cytotoxicity may be in comparison to a control culture where there are no immune cells or agents comprising an Fc domain, a control culture where there are immune cells but no agents comprising an Fc domain, or a control culture where there are agents comprising an Fc domain but no immune cells.

Any cell may be used as a target cell (i.e., the first target cell or the second target cell) in the in vitro assays contemplated herein. As a non-limiting example, the target cell may be a cell that displays elements of a proliferative disorder (e.g., a cancer cell). For example, the cells used may be one or more of the following: A-253, A-375, A-431, A-549, Colo205, Detroit-562, FaDu, HCC1954, HCC827, HepG2, HL-60, HT29, HuNS1, JEG-3, K562, KB, KGla, LoVo, LS411N, Malme-3M, MDA-MB-231, MM1-S, MV411, NCI MCF7, NCI-H1299, NCI-H1437, NCI-H1563, NCI-H1573, NCI-H1975, NCI-H2110, NCI-H446, NCI-H508, NCI-H661, NCI-H929, NCI-N87, NIH OVCAR3, OV90, PA-1, RKO, RPMI8226, SCC25, SCC9, SK-BR-3, SK-OV-3, SNU-C1, SW1417, SW48, SW626, or U266B1. The cells used may be Daudi cells or Raji cells.

As a non-limiting example, the immune cells may be washed (e.g., rinsed one or more times) in order to separate the immune cell from the first target cell and the first agent.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (1RL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Examples

### Example 1: Combination of Anti-Cancer Antibodies Targeting Different Epitopes of Same Cancer Antigen

Results obtained from the instant studies indicate that the combination of at least two anti-cancer antibodies and immune cells expressing chimeric activators may more efficiently recognize and induce cytotoxicity of cancer cells that have lower expression of the target cell antigen.

### Anti-Cancer Antibody Cell Binding

The ability of the anti-cancer antibodies to bind to target cancer cells was assessed by incubating various cancer cell lines with the anti-cancer antibodies, alone or in combination. The cell lines tested included A-253, A-375, A-431, A-549, Colo205, Detroit-562, FaDu, HCC1954, HCC827, HepG2, HL-60, HT29, HuNS1, JEG-3, K562, KB, KGla, LoVo, LS411N, Malme-3M, MDA-MB-231, MM1-S, MV411, NCI MCF7, NCI-H1299, NCI-H1437, NCI-H1563, NCI-H1573, NCI-H1975, NCI-H2110, NCI-H446, NCI-H508, NCI-H661, NCI-H929, NCI-N87, NIH OVCAR3, OV90, PA-1, RKO, RPMI8226, SCC25, SCC9, SK-BR-3, SK-OV-3, SNU-C1, SW1417, SW48, SW626, and U266B1. To test targeting of HER2-expressing cancer cells, the cell lines were incubated with the anti-HER2 antibodies Trastuzumab and Pertuzumab, alone or in combination, at various antibody concentrations. To test targeting of EGFR-expressing cancer cells, the cell lines were incubated with the anti-EGFR antibodies ABT-085, Cetuximab, or a combination of anti-EGFR antibodies (EGFR A and EGFR B), at various antibody concentrations.

The cells were then washed with phosphate buffered saline (PBS) incubated with a secondary antibody. Cell staining was measured using a flow cytometer and is a measure of surface expression of the exemplary target cancer antigens (HER2 or EGFR).

Jurkat Cell Activation with Combination of Antibodies and T-Cells Expressing ACTR

The ability of anti-cancer antibodies to activate Jurkat cells expressing chimeric receptors was analyzed in a reporter assay in Jurkat cells that is reflective of Jurkat cell activation. Jurkat cells were transduced with lentivirus encoding firefly luciferase downstream of a minimal CMV promoter element and tandem repeats of the nuclear factor of activated T-cells (NFAT) consensus binding site. In this cell line, upregulation of NFAT transcription factors results in binding to the transcriptional response elements and subsequent expression of luciferase, which is monitored by measuring light produced following luciferase cleavage of its substrate luciferin.

Jurkat cells with the NFAT reporter system (Jurkat-N) were transduced with retrovirus expressing ACTR (Jurkat-N-ACTR). Jurkat cells with the NFAT reporter system, but lacking ACTR expression (Jurkat-N-Mock) were used as a negative control. To assess Jurkat-N activation by anti-HER2 antibodies, Jurkat N-ACTR and Jurkat-N-Mock cells were mixed with target cancer cells bound by Trastuzumab, Pertuzumab, or the combination of Trastuzumab and Pertuzumab, at various antibody concentrations. To assess Jurkat-N activation by anti-EGFR antibodies, Jurkat N-ACTR and Jurkat-N-Mock cells were mixed with target cancer cells bound by ABT-085, Cetuximab, or the combination of anti-EGFR antibodies (EGFR A and EGFR B), at various antibody concentrations. Following incubation, Bright-Glo reagent (100 μL, Promega; Madison, Wis.) was added to lyse the cells and add the luciferin substrate. Reactions were incubated for 10 minutes in the dark and luminescence was measured using an EnVision Multi-label plate reader (Perkin-Elmer; Waltham, Mass.).

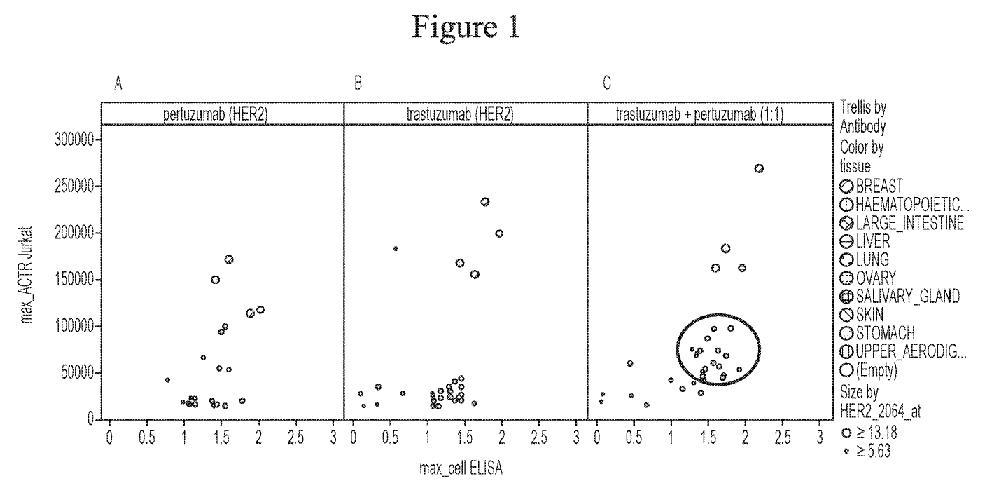

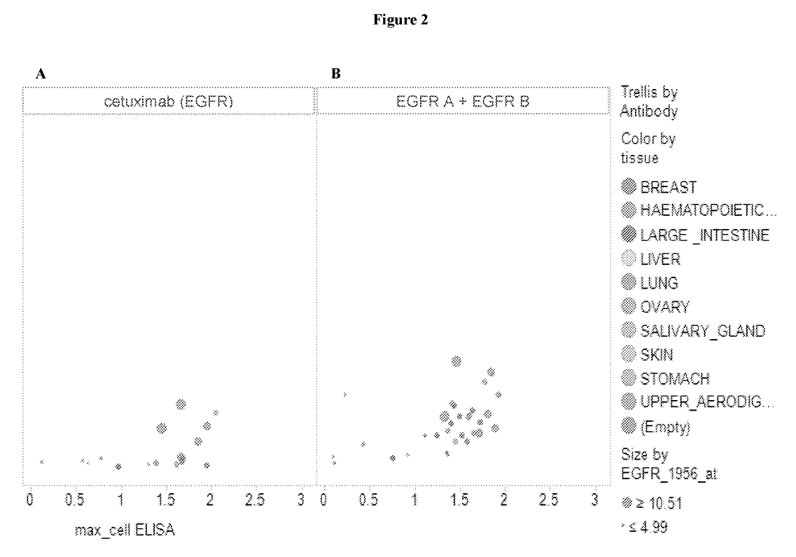

The cell staining data was plotted versus the Jurkat cell activation data. FIGS. 1 and 2. In the case of targeting the cancer antigen HER2 as well as targeting the cancer antigen EGFR, incubation of the cells with one anti-cancer antibody led to Jurkat cell activation when the target cells showed relatively a high level of antigen expression. However, incubation of the cells with the combination of antibodies resulted in increased Jurkat cell activation when the target cells showed lower expression of the target cell antigen, relative incubation of the cells with a single antibody.

### Cytotoxicity of Cancer Cells Induced by T-Cells Expressing a CTR and Cytokine Release

Gamma-retrovirus encoding an expression construct (e.g., SEQ ID NO: 1) that comprises the CD16 extracellular domain, linked to the 4-1BB costimulatory domain, and the T-cell receptor CD3z intracellular domain (CD16V-41BB-CD3z, was generated via routine recombinant technology. See, e.g., Kudo et al., (2014), Cancer Res. 74, 93-103; and WO2015058018, the relevant disclosures therein are hereby incorporated by reference. This retrovirus was used to infect primary human T-cells for generating cells that express ACTR on their cell surface. These cells were used in cytotoxicity assays with target cells bound by anti-cancer antibodies. Mock T-cells that were not transduced with ACTR retrovirus were used as a control in this experiment.

T-cells expressing ACTR and target cells bound by anti-cancer antibodies (single antibody or combination of at least two antibodies) were incubated together. The percent cytotoxicity was determined by quantifying the live vs dead cells in the population of target cells.

Cytokine release assays were also performed to evaluate cytokines secreted in response to the T cells expressing ACTR activation by target cells bound by anti-cancer antibodies.

Briefly, ACTR T cells combined with either HER2 targeting Trastuzumab or Pertuzumab exhibited potent cytotoxic activity, cytokine response and proliferation on a HER2 amplified tumor cell lines. ACTR activity was specific to antibody treated cells, and had little activity on HER2 low or negative tumor lines. Furthermore, the effectiveness of ACTR T cells in a non-amplified HER2 setting was tested in the presence of a combination of Trastuzumab and Pertuzumab. This multi-antibody combination increased the cytotoxicity of ACTR T-cells on non-amplified HER2 expressing cancer cell lines, whereas Trastuzumab or Pertuzumab as single antibody combinations with ACTR had little effect.

### Example 2: Co-Use of ACTR and Anti-Cancer Antibodies Targeting Different Cancer Antigens

To determine whether T-cells expressing ACTR could be combined with a diverse array of tumor-targeting antibodies against B-cell malignancies, cell lines derived from B-cell lymphomas or multiple myeloma were used. It was found that T cells expressing ACTR could be activated with antibodies against a diverse set of B-cell targets, including CD19, CD20, CD22, CD38, and CS1.

Jurkat Cell Activation with Combination of Antibodies and T-Cells Expressing ACTR

The ability of anti-cancer antibodies to activate Jurkat cells expressing chimeric receptors was analyzed using the NFAT-luciferase reporter assay in Jurkat cells that is reflective of Jurkat T cell activation. Jurkat cells were transduced with lentivirus encoding firefly luciferase downstream of a minimal CMV promoter element and tandem repeats of the nuclear factor of activated T-cells (NFAT) consensus binding site. In this cell line, activation of NFAT transcription factors results in binding to the transcriptional response elements and subsequent expression of luciferase, which is monitored by measuring light produced following luciferase cleavage of its substrate luciferin.

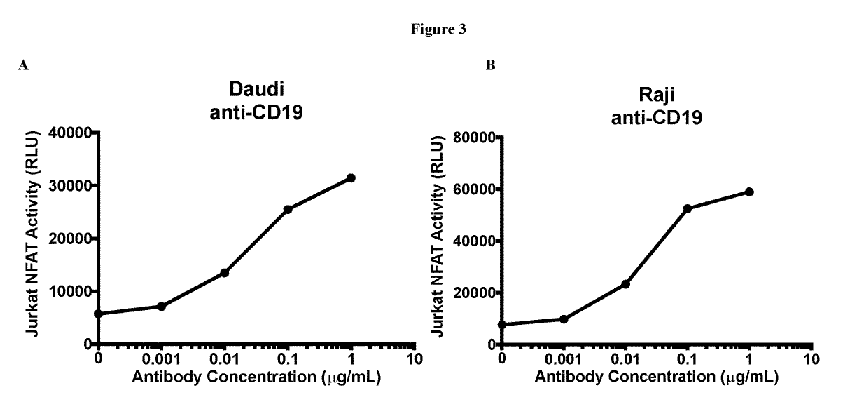

Jurkat cells with the NFAT reporter system (Jurkat-N) were transduced with retrovirus expressing ACTR (Jurkat-N-ACTR). To assess Jurkat-N activation by anti-CD19 antibodies in the presence of CD19-expressing cells, Jurkat N-ACTR cells were mixed at a 1:1 ratio with target CD19-expressing Daudi cells or CD19-expressing Raji cells and varying concentrations of the anti-CD19 antibody MOR208 (0-1 μg/mL). Reactions were incubated for 5 hr in CO₂ (5%) incubator at 37° C. Following incubation, Bright-Glo reagent (100 μL, Promega; Madison, Wis.) was added to lyse the cells and add the luciferin substrate. Reactions were incubated for 10 minutes in the dark and luminescence was measured using an EnVision Multi-label plate reader (Perkin-Elmer; Waltham, Mass.). FIG. 3 shows that Jurkat N-ACTR cells were activated when combined with target cells expressing CD19 and anti-CD19 antibodies.

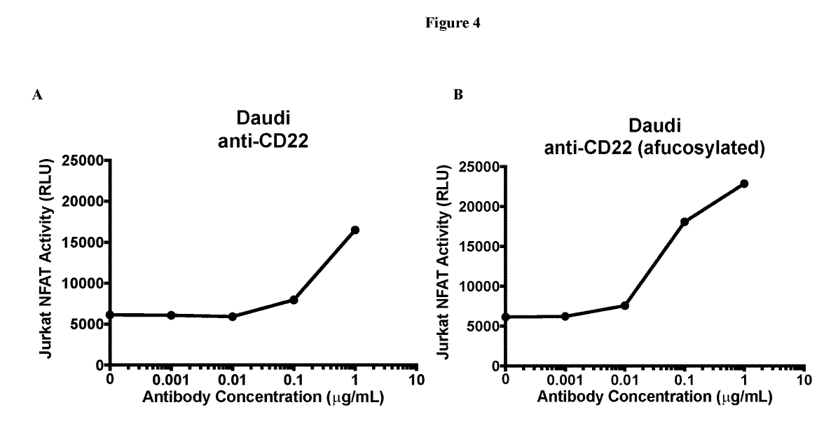

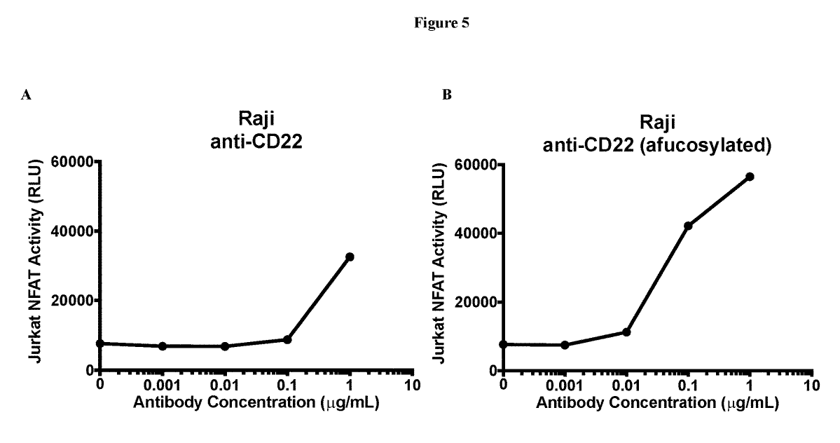

Similar experiments were performed to assess Jurkat-N activation by anti-CD22 antibodies in the presence of CD22-expressing cells. Jurkat N-ACTR cells were mixed at a 1:1 ratio with target CD22-expressing Daudi cells or CD22-expressing Raji cells and varying concentrations of the anti-CD22 antibody Epratuzumab (0-1 μg/mL), which was either fucosylated or afucosylated. FIGS. 4 (panel A) and 5 (panel A) show that Jurkat N-ACTR cells were activated when combined with target cells expressing CD22 and the anti-CD22 antibody Epratuzumab. The extent of Jurkat N-ACTR cell activation was enhanced when the Jurkat N-ACTR cells were combined with target cells expressing CD22 and afucosylated Epratuzumab (see panel B of FIGS. 4 and 5).

### Cytokine Release Induced by T-Cells Expressing ACTR

Gamma-retrovirus encoding an expression construct (e.g., SEQ ID NO: 1) was generated via routine recombinant technology and used to infect primary human T-cells for generating cells that express ACTR on their cell surface. T-cells expressing ACTR and target cells expressing CD19 (RL, Raji, or Namalwa tumor cells) were incubated together at a ratio of 2:1 (effector cells/ACTR-expressing T cells to target cells) with varying concentrations of anti-CD19 antibodies (MOR208). Similarly, T cells ACTR and target cells expressing CD22 (RL, Raji, or Namalwa tumor cells) were incubated together at a ratio of 2:1 (effector cells/ACTR-expressing T cells to target cells) with varying concentrations of anti-CD22 antibodies (afucosylated Epratuzumab). Supernatants were analyzed for IFNγ using the Meso Scale Discovery V-Plex Proinflammatory Panel 1 Kit (Human, K15049D-1) following 24 hr incubation. Briefly, the Proinflammatory Calibrator Blend, Detection Antibody Solution, and Read Buffer were prepared according to manufacturer protocol. Co-culture supernatants were diluted 1:200 in RPMI 1640 media supplemented with 10% fetal bovine serum. 50 μL of diluted sample or calibrator was added to the MSD plate, and the plate was sealed and incubated on a shaker at 600×g for 2 hours in the dark. The plate was washed three times with 150 μL Phosphate Buffered Saline containing 0.05% Tween-20. 25μL of detection antibody solution was added to the plate, and the plate was sealed and incubated on a shaker at 600×g for 2 hours in the dark. The plate was washed three times with 150 μL Phosphate Buffered Saline containing 0.05% Tween-20. Read Buffer (150 μL) was added to the plate and the plates were run on the MSD Quickplex SQ 120.

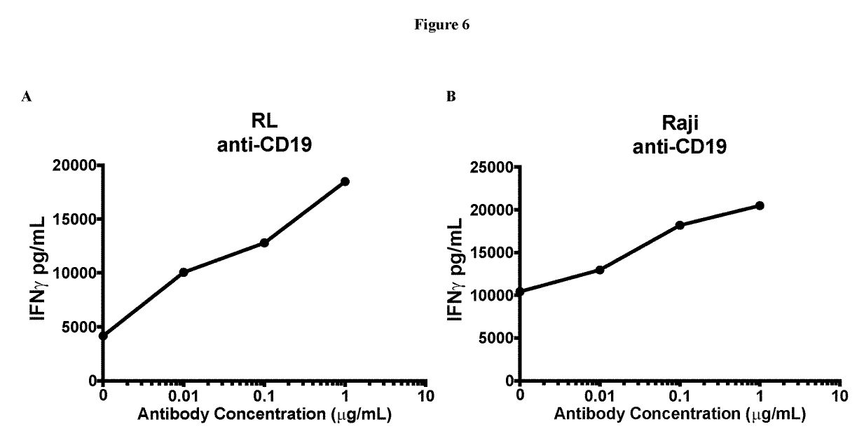

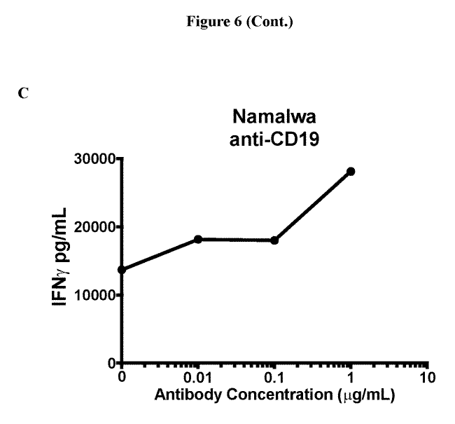

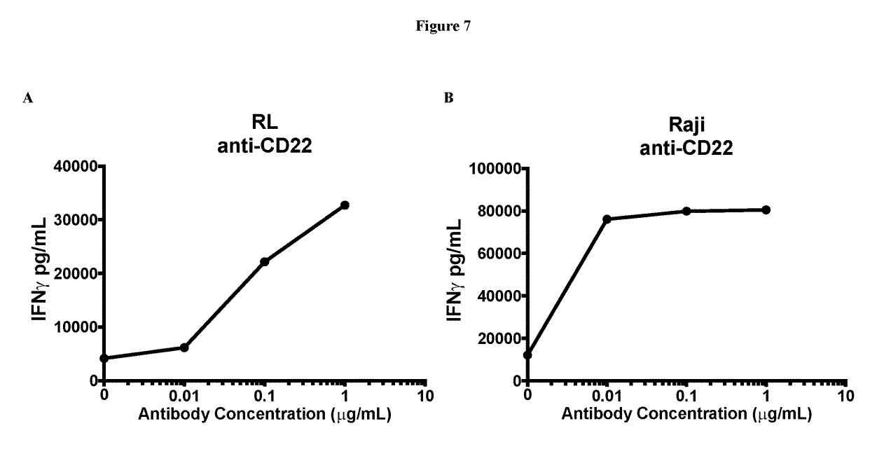

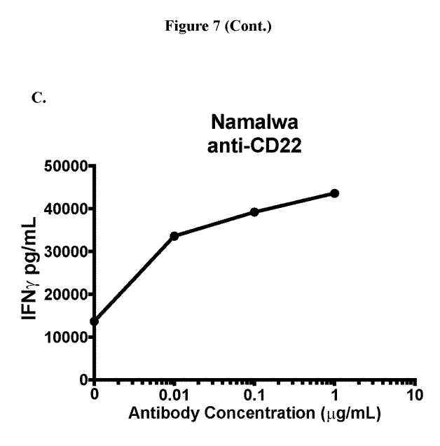

As shown in FIGS. 6 and 7, both CD19- or CD22-expressing target cells induce increasing ACTR T cell IFNγ release when in the presence of increasing concentrations of anti-CD19 or anti-CD22 antibodies.

### Cytotoxicity of Cancer Cells Induced by T-Cells Expressing a CTR and Cytokine Release

To assess activation and effector function of ACTR-T cells in the presence of target cells expressing two cancer antigens, RL cells expressing CD19 and CD22 were labeled with Cell Trace Violet. Labeled target cells were incubated with ACTR-expressing T cells, at a ratio of 2:1 effector cells (ACTR-expressing T cells) to target cells (RL cells) with 1 μg/mL of the anti-CD19 antibody MOR208, the anti-CD22 antibody Epratuzumab (afucosylated), or the combination of the anti-CD19 antibody and the anti-CD22 antibody. After incubating for 24 hours, the cytotoxicity was determined by quantifying the live cells (CellTrace Violet positive cells) by flow cytometry. The percent cytotoxicity was calculated as the percentage of RL cells remaining relative to control samples no containing T cells-expressing ACTR. The culture supernatants were harvested for analysis, and the amount of IFNγ secreted was measured by MSD electrochemiluminescence, as described above.

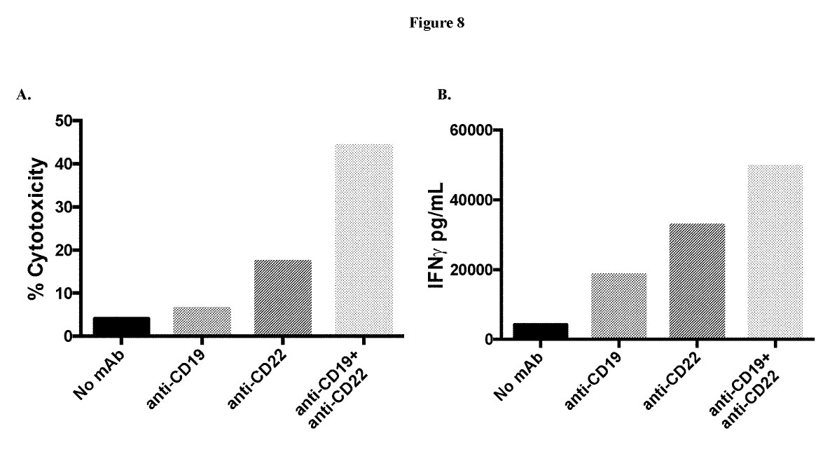

ACTR T cells combined with CD19 targeting MOR208 and CD22 targeting Epratuzumab exhibited enhanced cytotoxic activity and cytokine release in response to CD19- and CD22-expressing target cells. FIG. 8.

### Example 3: Efficient Targeting of HER-2 Positive Cancers by Antibody-Coupled T Cell Receptor (ACTR) Engineered Autologous T Cells

ACTR T-cells were shown to mediate potent activity against HER-2 positive tumor cells in the presence of a combination of HER-2 targeting antibodies trastuzumab and pertuzumab.

### Her-2 Expression in Cell Lines

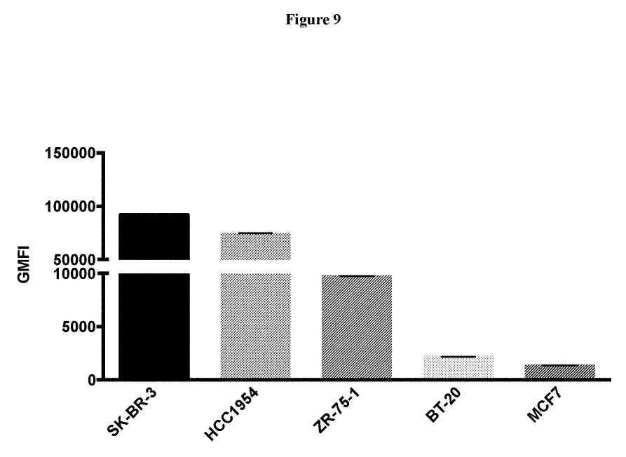

The levels of HER-2 expression on various tumor lines were measured by flow cytometry (Attune Life Technologies) with a PE anti-human HER-2 antibody. The values reported are the Geometric Mean Fluorescent Intensity values of the staining (see FIG. 9). SK-BR-3 and HCC1954 are considered HER-2 amplified cell lines (with high expression levels of HER2) while ZR-75-1, BT-20 and MCF7 are considered non-amplified cell lines (with low expression levels of HER2).

Correlation of NFAT Jurkat Activation with HER-2 Expression

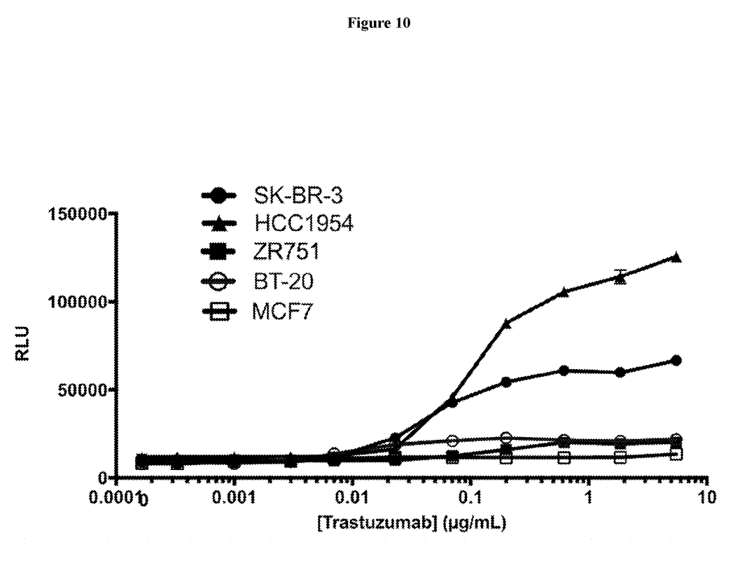

Activation of ACTR in the presence of HER2 and a HER2-specific antibody, trastuzumab, was measured with a Jurkat-NFAT reporter line stably expressing ACTR. The cells were lysed and activation was measured by the emission of luminescence 5-hours post setup using the Promega BrightGlo luminescence kit on the Perkin Elmer Envision instrument. Activation was measured by the emission of luminescence 5-hours post-setup. The level of activation, measured in relative luminescence units (RLU), is dependent on the HER2 expression level and antibody concentration (see FIG. 10).

### Mediation of ACTR Cytotoxicity by Target-Antibody Pairing

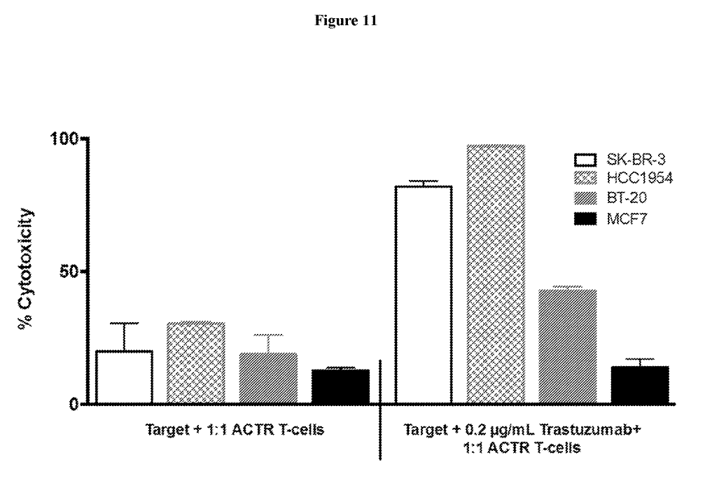

The cytotoxicity of HER2+ cell lines mediated by primary ACTR T-cells in combination with trastuzumab was measured at 48 hours. Target cells expressing luciferase were plated with or without antibody in the presence of ACTR T-cells at a 1:1 E:T ratio. The cells were lysed and the cytotoxicity was measured using the Promega Bright Glo Kit and the plates were read on the Perkin Elmer Envision. Cytotoxicity was measured as a loss of luminescence compared to target alone controls. ACTR T-cells exhibited robust cytotoxicity on amplified HER-2 lines, and moderate to no cytotoxicity on non-amplified lines (see FIG. 11).

### Pro-Inflammatory Cytokine Response of ACTR T-Cells on HER-2 Amplified Cell Lines

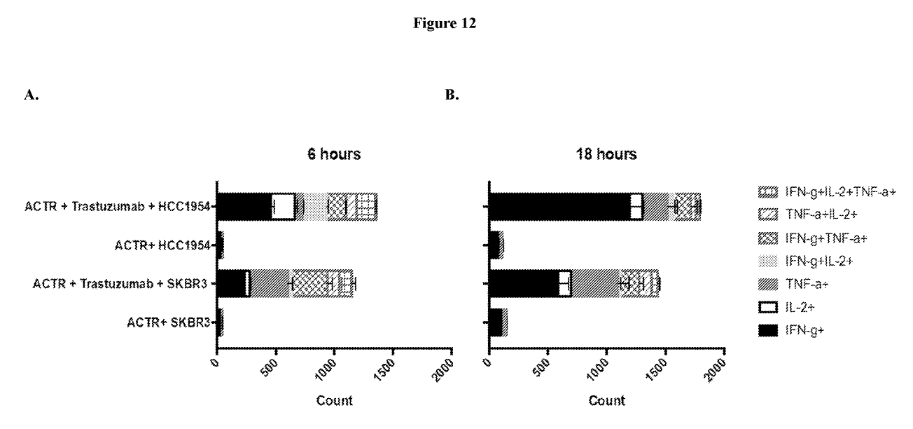

Cytokine (IFN-g, IL-2 and TNF-α) production from ACTR T-cells was measured through intracellular cytokine staining at 6 and 18 hours in the presence of target (HER2), with and without 1 μg/mL of trastuzumab. The total cytokine response from ACTR T-cells was plotted and the subsets of the response are expressed in the graph as parts of the whole. The overall cytokine response increased from 6 to 18 hours and the induction of a robust pro-inflammatory response only occurred in the presence of a target-specific antibody (FIG. 12).

### Proliferation of Primary ACTR T-Cells on HER-2 Amplified and Non-Amplified Cell Lines

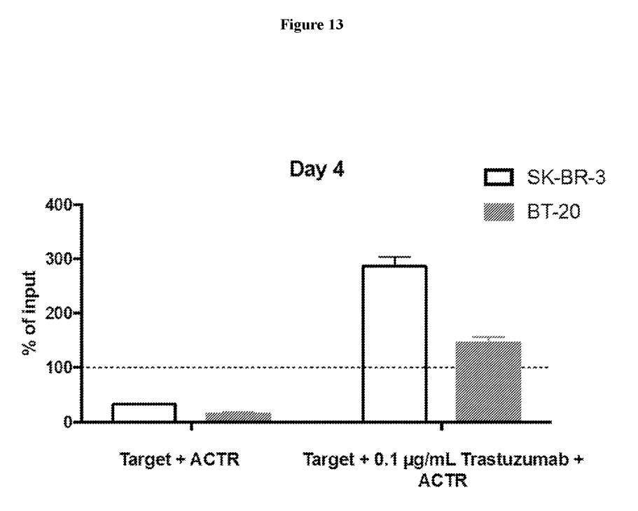

The antibody and target-specific proliferation of ACTR T-cells in the presence of target (HER2) and trastuzumab was determined in a four-day flow-based proliferation assay. The number of ACTR T-cells was determined by flow cytometry on Day 0 and Day 4 with an APC anti-human CD16 antibody. The values are reported as the percent of input ACTR T cells (see FIG. 13). ACTR T-cells proliferate more rapidly on HER-2 amplified SK-BR-3 cells as compared to the non-amplified BT-20 cells, indicating that the ACTR response is specific in the presence of a targeting antibody and correlates to the HER-2 expression on a tumor.

Cytotoxicity in Cell Lines in which HER-2 is not Amplified

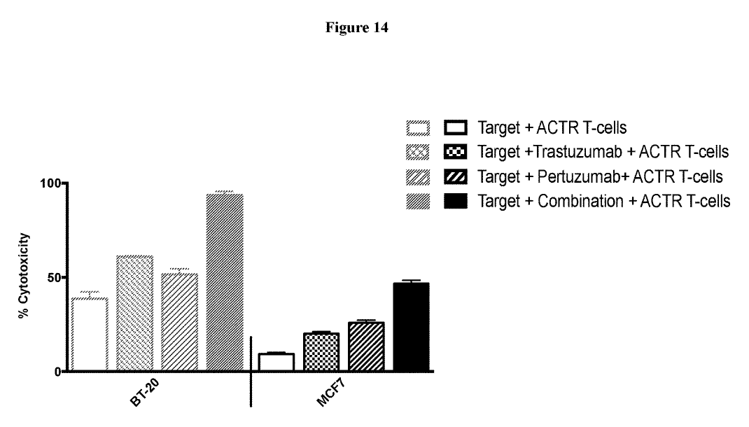

The effect of the trastuzumab and pertuzumab (both singly and in combination) on ACTR cytotoxicity was examined in a 48-hour luciferase based cytotoxicity assay at a 2:1 E:T ratio. Cytotoxicity was measured as the loss of luminescence from the target line. The addition of both antibodies at 0.1 μg/mL increased ACTR function when compared to either antibody alone on the non-HER-2-amplified BT-20 and MCF7 tumor lines (see FIG. 14).

Pro-Inflammatory Response in Cell Lines in which HER-2 is not Amplified

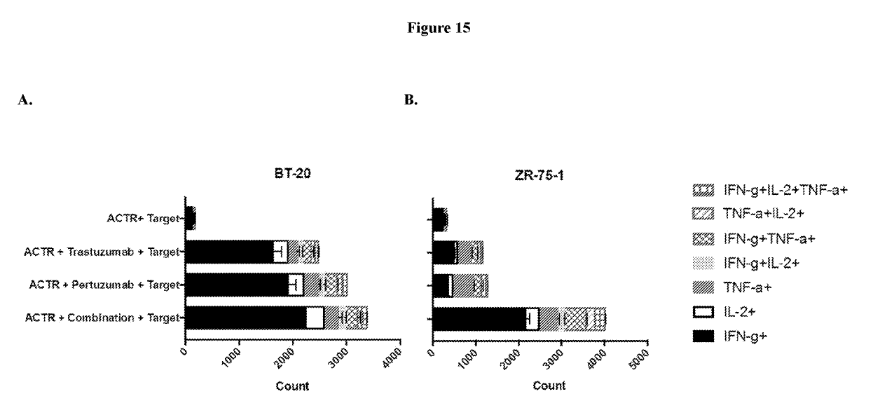

The effect of trastuzumab and pertuzumab on the IFN-g, IL-2 and TNF-α cytokine production of ACTR T-cells was explored on two non-amplified HER-2 cell lines (BT-20 and ZR-75-1) at 18 hours. The total cytokine response from ACTR T-cells was plotted and the subsets of the response are expressed as parts of the whole. The combination of the two antibodies resulted in greater overall cytokine production compared to each antibody alone, with the greatest differential seen in the results from the ZR-75-1 cell line. The results from these experiments are shown in FIG. 15.

Proliferation in Cell Lines in which HER-2 is not Amplified

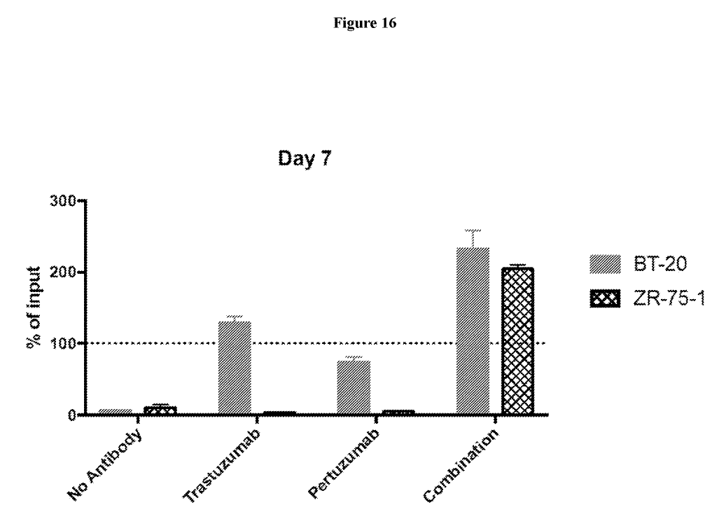

The effect of the trastuzumab/pertuzumab combination on proliferation of ACTR T-cells was explored in a seven day single stimulation assay. The number of ACTR T-cells was determined on Day 0 and Day 7 by flow cytometry using an APC anti-human CD16 antibody. The values are reported as a percentage of input ACTR T-cells. The antibody combination greatly increased proliferation on the non-amplified BT-20 and ZR-75-1 cell lines and resulted in a two- to three-fold increase in proliferation by Day 7, as compared with the proliferation of BT-20 and ZR-75-1 cells in the presence of either trastuzumab or pertuzumab alone. The results of the proliferation experiments using the non-amplified lines can be seen in FIG. 16.

### PD-L1 Expression on HER-2 Positive Cell Lines

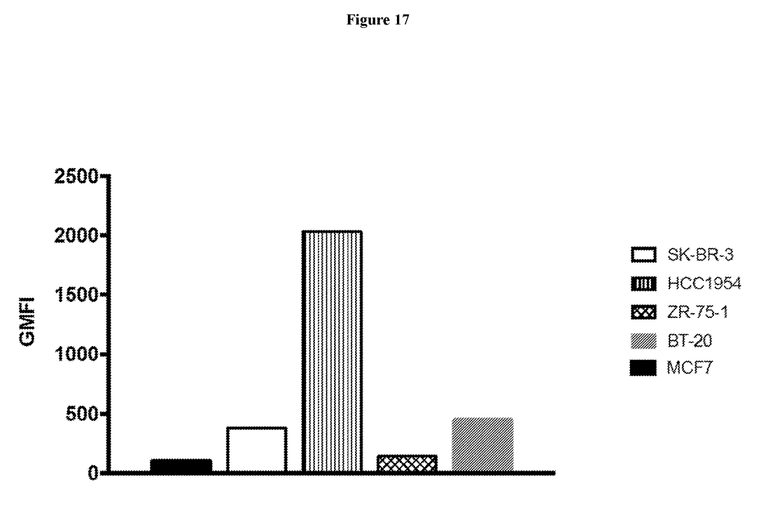

PD-L1 expression on HER-2 positive tumor lines was evaluated by flow cytometry with an anti-human PD-L1 antibody. The values reported in FIG. 17 are the GMFI of the staining. Cell line HCC1954 demonstrates PD-L1 levels above isotype staining.

### Cytotoxicity of ACTR T-Cells on a HER-2+/PD-L1+ Spheroid

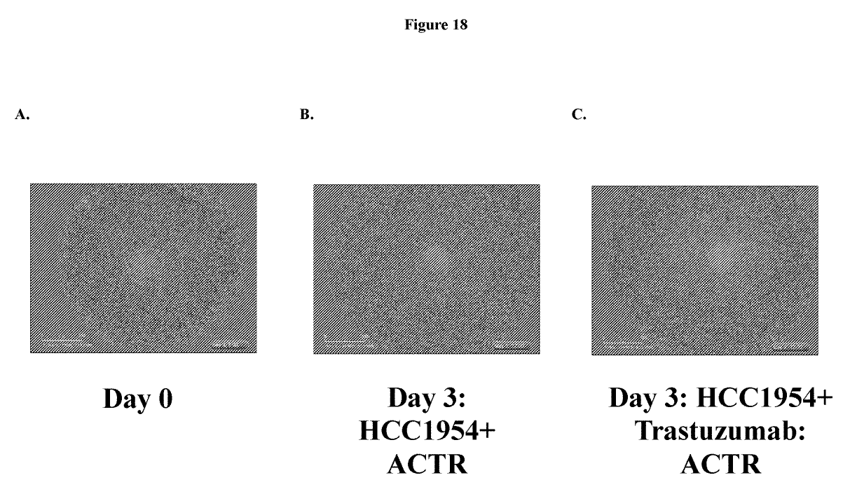

The invasive ability of ACTR T-cells was examined through generation of HER-2+/PD-L1+ HCC1954 spheroids, staining with CellTracker Red, and subsequent imaging to visualize cell death over a three-day period using the Incucyte® system. The T-cells were added at a 4:1 E:T ratio. Only in the presence of trastuzumab were ACTR T-cells able to efficiently invade and kill the HCC1954 target (see FIG. 18). The limited effect on the tumor in the absence of an antibody demonstrates the specificity of ACTR T-cells and suggests the potential for therapeutic effect in a solid tumor setting.

Based on the above results, ACTR T-cells have been shown to mediate potent activity against HER-2 positive tumor cells in the presence of HER-2 targeting antibodies, for example, trastuzumab and/or pertuzumab. The combination of both trastuzumab and pertuzumab resulted in robust antigen-specific activity, compared to either antibody alone, on non-amplified HER-2-positive cell lines.

ACTR T-cells were also shown to be capable of invading a HER-2+/PD-L1+ HCC1954 spheroid model in vitro, suggesting that ACTR T-cells are able to have therapeutic potential in a solid tumor setting.

### Example 4: High Throughput Triage of the ACTR Platform

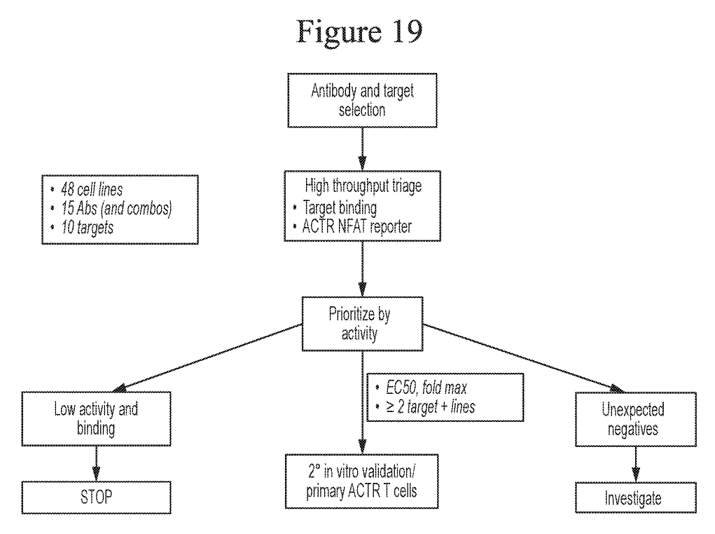

The ACTR platform was evaluated as shown in FIG. 19 using a broad panel of tumor-targeting antibodies against multiple tumor cell lines. The high-throughput triage characterized ACTR activity in a Jurkat-NFAT-luciferase reporter assay and benchmarked activity relative to target binding in a cell-based ELISA.

### Methods

### ACTR Jurkat-NFAT-Luciferase Reporter Assay

Tumor cell lines were treated with a dose titration of tumor-targeting antibodies and cultured with Jurkat cells transduced with the ACTR-NFAT-luciferase reporter at an E:T ratio of 1:1. After a 5 hour incubation, luminescence was measured using the BriteLite Plus reporter gene assay system (Perkin Elmer).

### Cell-Based ELISA

Tumor cell lines were blocked with goat serum, then stained with a dose titration of tumor-targeting antibodies. Antibodies bound to target cells were detected with an HRP-conjugated goat anti-human secondary antibody and developed with the TMB substrate kit (Pierce).

### IL-2 Release Assay

Tumor cell lines were treated with a dose titration of tumor-targeting antibodies and cultured with primary ACTR T cells at an E:T of either 2:1 or 1:1. Cell culture supernatants were collected after 24 hours, and IL-2 was measured using the V-PLEX Human IL-2 kit (Meso Scale Discovery).

### ACTR Proliferation Assay

Tumor cell lines were treated with a dose titration of tumor-targeting antibodies and cultured with primary ACTR T cells at an E:T of 1:1. Fresh medium was added to the co-culture every 2-3 days. After 6 days, cultures were stained with a CD16-specific monoclonal antibody, and total ACTR cells counts were measured by flow cytometry.

### Results

### ACTR Activity Against Multiple Targets

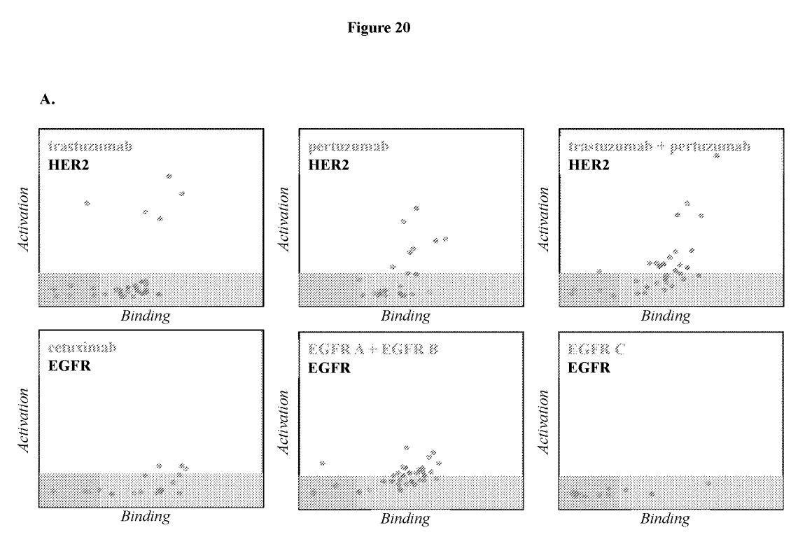

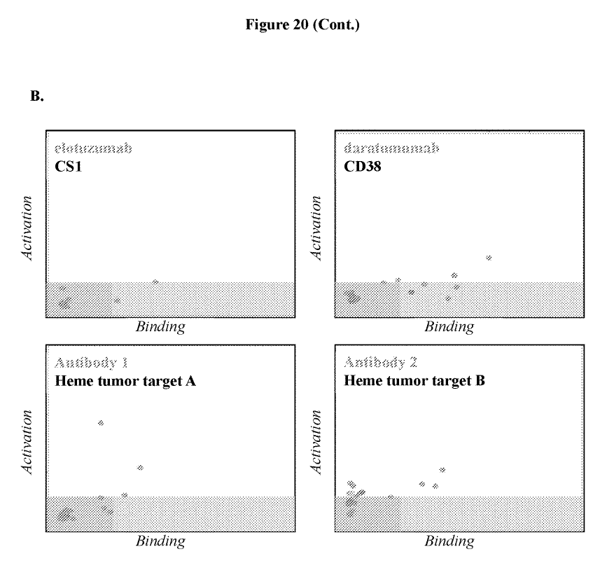

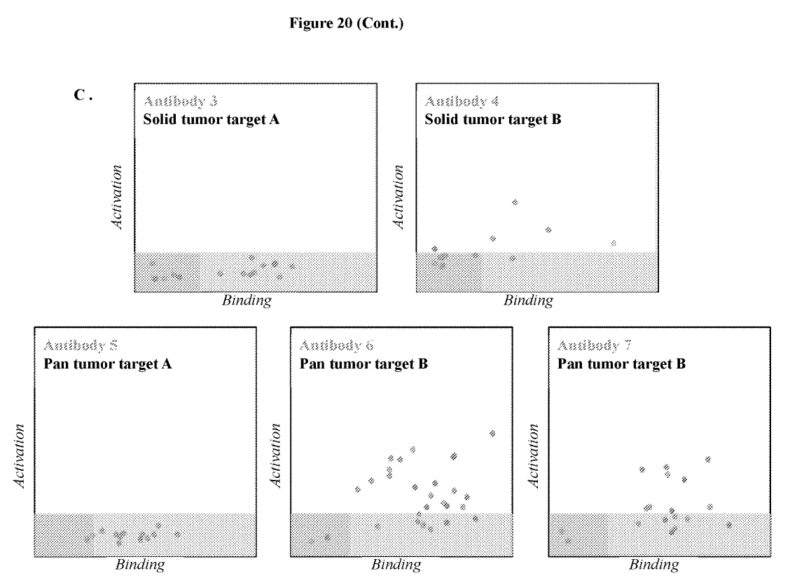

Binding to target cells and ACTR activity in a Jurkat-NFAT-luciferase reporter assay were characterized for a panel of tumor-targeting antibodies across hematological and solid tumor indications. A subset of example data are shown in FIG. 20 (panels A-C), depicting the relation between target binding and ACTR activation across antibodies and targets. Each point represents an individual target cell line. Binding to target cells was necessary but not sufficient to trigger ACTR activation. Activity was confirmed with a number of marketed therapeutic antibodies including: trastuzumab, pertuzumab, and cetuximab, including enhanced activity with combinations to the same target. Broad activity was observed against multiple targets covering both hematological and solid malignancies. Anti-HER-2 antibodies (trastuzumab; pertuzumab; and the combination of trastuzumab and pertuzumab) and anti-EGFR antibodies (cetuximab; anti-EGFR A and anti-EGFR B; and anti-EGFR C) are shown in FIG. 20, panel A. Anti-CS1 antibodies (elotuzumab), anti-CD38 antibodies (daratumumab), and antibodies against two different Heme tumor targets (A and B) are shown in FIG. 20, panel B. Antibodies against solid tumor targets (A and B) and Pan tumor targets (A and B) are shown in FIG. 20, panel C.

Multi-Antibody Combination Activity Identified with ACTR

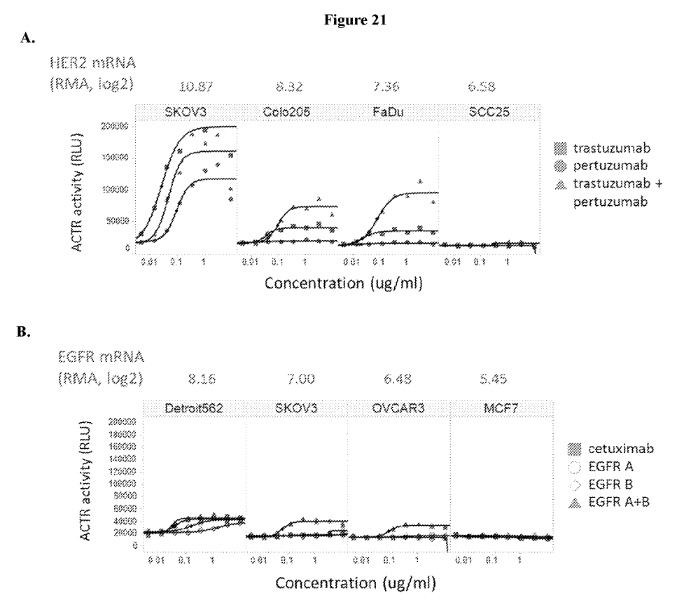

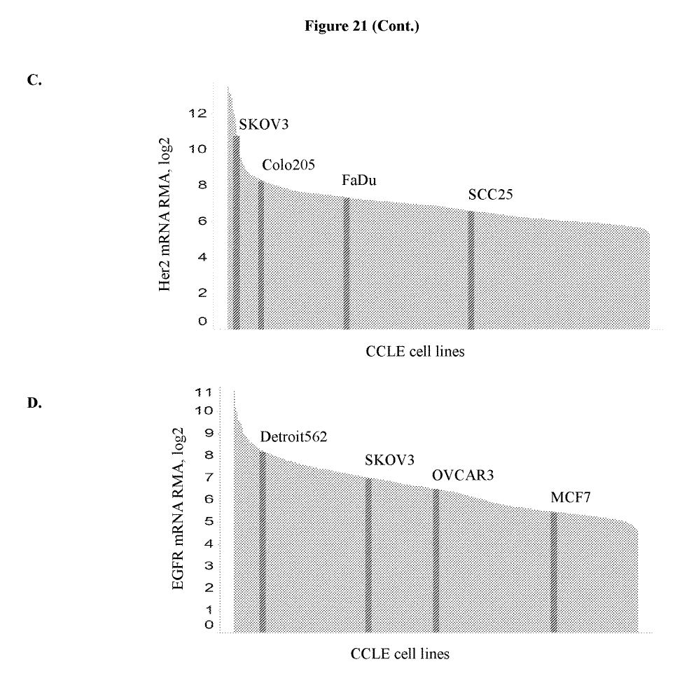

Targeting multiple epitopes through antibody combinations enhanced ACTR activity. Single antibody treatment activated ACTR at high target expression levels, but at lower levels, a combination effect was evident. This was demonstrated with anti-Her2 antibodies, trastuzumab and pertuzumab, (see FIG. 21, panel A) and with anti-EGFR antibodies, A and B against non-overlapping epitopes (see FIG. 21, panel B). Relative gene expression levels of Her2 and EGFR for this set of tumor cell lines are depicted in FIG. 21, panels C and D (respectively). See (Barretina et al. Nature. 2012; 483:603-7).

### Effective Discrimination Between Active and Inactive Combination Partners Using ACTR

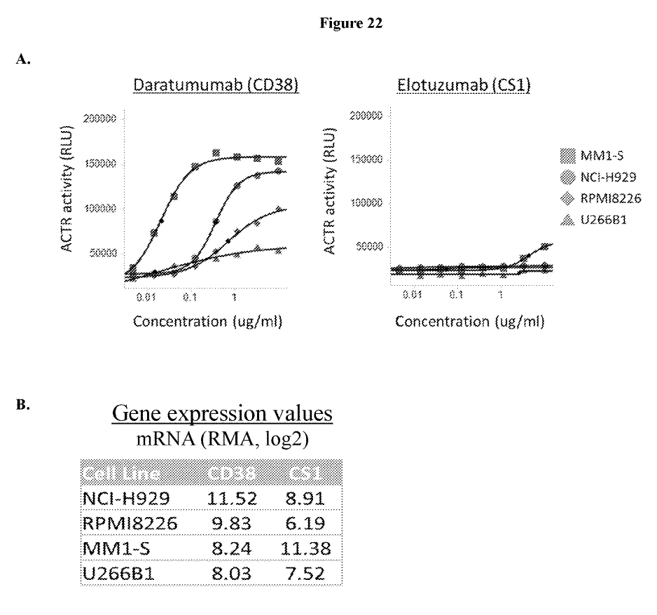

Despite reported ADCC activity (Tai et al. Blood. 2008; 112:1329-37), elotuzumab was not an effective combination partner with ACTR as there was little to no activity against CS1 in a panel of multiple myeloma cell lines. By contrast, daratumumab demonstrated ACTR activity against CD38 in the same panel of multiple myeloma cell lines (see FIG. 22, panel A). This difference in activity was observed despite the high level of gene expression for both CS1 and CD38 (see FIG. 22, panel B). The high throughput triage was able to successfully identify a range of activities across this panel of tumor-targeting antibodies and to therefore identify effective and ineffective partners for potential combination with ACTR.

### Correlation of Activity in Primary ACTR T Cells

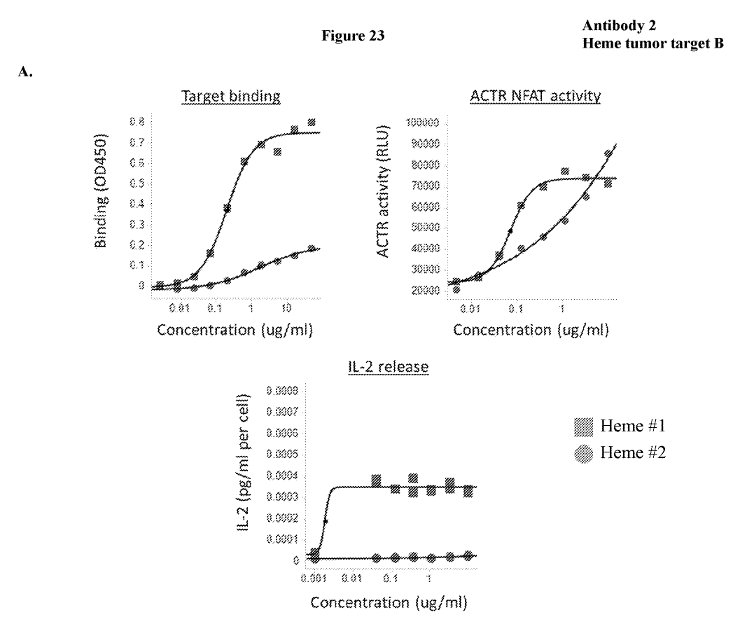

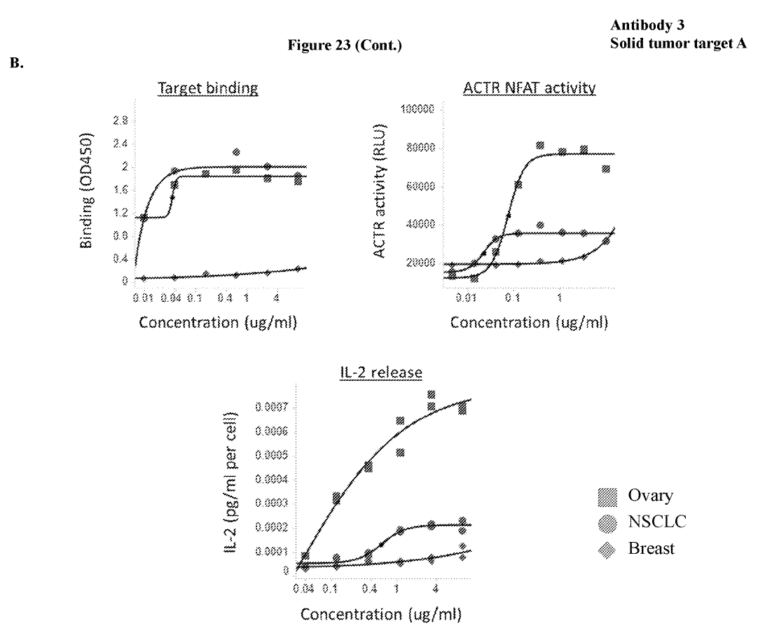

While target binding was necessary but not sufficient to drive ACTR activity, there was a stronger correlation between Jurkat-NFAT-luciferase activity and IL-2 release from primary ACTR T-cells. Examples of this correlation were observed for a hematological tumor target (FIG. 23, panel A) and a solid tumor target (FIG. 23, panel B). In each case, differential activity in ACTR NFAT activity correlated with a comparable difference in IL-2 release. This demonstrates the ability of this high throughput triage to predict primary T-cell responses.

Identification and Characterization of Active Combination Partners with ACTR

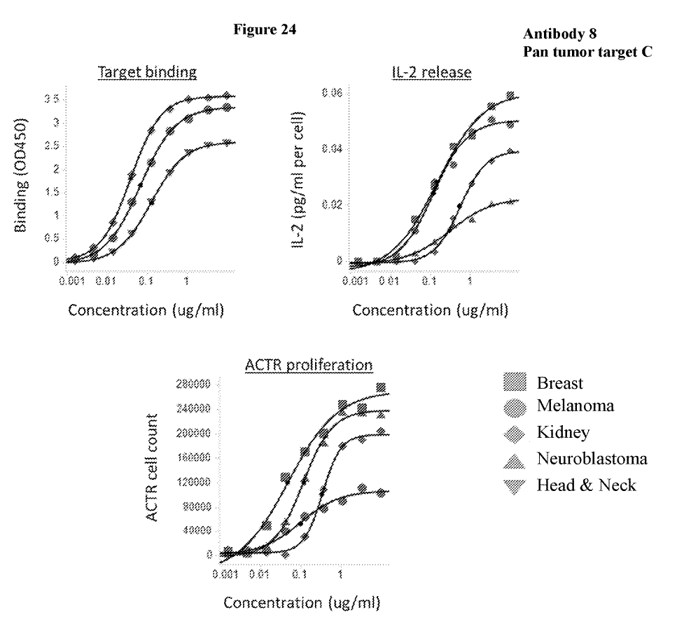

The high throughput triage identified a range of activities across this panel of tumor-targeting antibodies. ACTR activity was identified using a pan tumor targeting antibody and further characterized in secondary in vitro assays with primary ACTR T-cells. The activity was confirmed across a panel of tumor cell lines covering a broad range of indications (see FIG. 24). Based on the level of IL-2 response and ACTR T-cell proliferation, the high throughput triage has identified a target warranting further investigation.

A viable high throughput platform for screening ACTR activity across multiple antibodies spanning several indications was established. Using this platform, the potency of tumor-targeting antibodies was investigated; individual tumor targets were screened to identify active antibodies and combinations; and activity and expression relationships were defined through screening a large number of cell lines. In addition, the high throughput platform has been able to identify multi-antibody combination activity. The adaptability of the ACTR platform relative to conventional scFv-based CAR technology greatly facilitated these simultaneous comparisons across targets and combinations, bypassing the need to generate and characterize multiple scFv-based constructs. These results provide further evidence for ACTR as a universal, chimeric receptor with activity across multiple antibodies and targets.

### Example 5: Targeting B-Cell Malignancies with Primary ACTR T Cells and Anti-CD19, Anti-CD20, and/or Anti-CD22 Antibodies

Gamma-retrovirus encoding an ACTR expression construct (e.g., SEQ ID NO: 67, a.k.a. ACTR707) was generated via routine recombinant technology and used to infect primary human T-cells for generating cells that express the ACTR on their cell surface. T-cells expressing the ACTR and Daudi target cells expressing CD19, CD20 and CD22 were incubated together at a ratio of 1:1 (effector cells/ACTR-expressing T cells to target cells) with varying concentrations of CD19, CD20, and/or CD22 antibodies. T-cells expressing a CAR targeting CD19 (CAR19) were used for comparison.

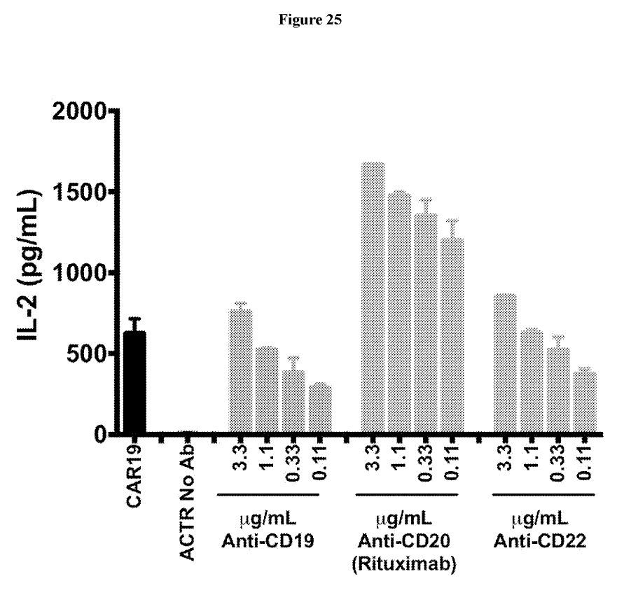

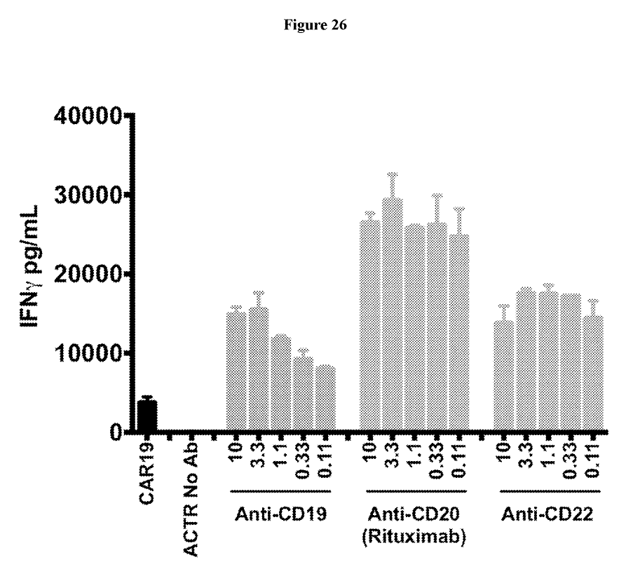

Supernatants were analyzed for IL-2 and IFNγ release using the Meso Scale Discovery V-Plex Proinflammatory Panel 1 Kit (Human, K15049D-1) following a 24 hour incubation. The Proinflammatory Calibrator Blend, Detection Antibody Solution, and Read Buffer were prepared according to manufacturer protocol. Co-culture supernatants were diluted 1:200 in RPMI 164015 media supplemented with 10% fetal bovine serum. 50 μL of diluted sample or calibrator was added to the MSD plate, and the plate was sealed and incubated on a shaker at 600×g for 2 hours in the dark. The plate was washed three times with 150 μL Phosphate Buffered Saline containing 0.05% Tween-20. 25 μL of detection antibody solution was added to the plate, and the plate was sealed and incubated on a shaker at 600×g for 2 hours in the dark. The plate was washed three times with 150 μL Phosphate Buffered Saline containing 0.05% Tween-20. Read Buffer (150 μL) was then added to the plate and the plates were run on the MSD Quickplex SQ 120. CD19-, CD20- or CD22-expressing Daudi target cells induce increasing ACTR T-cell IL-2 release and IFNγ release when in the presence of increasing concentrations of anti-CD19, anti-CD20, or anti-CD22 antibodies. Results are shown in FIG. 25 (IL-2) and FIG. 26 (IFNγ).

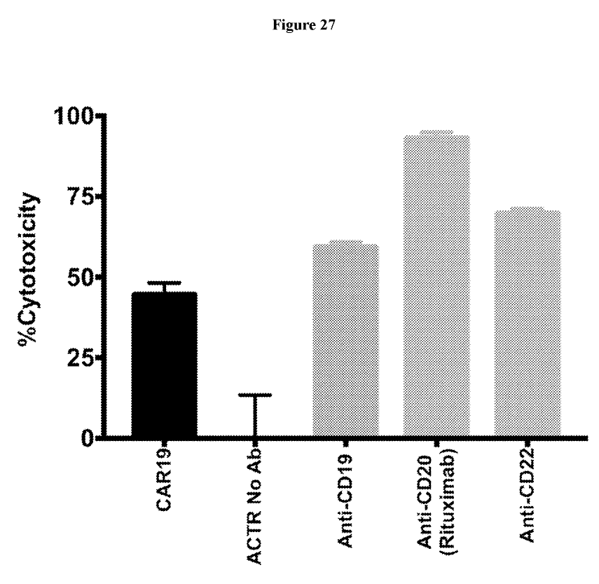

To assess activation and effector function of ACTR-expressing T-cells in the presence of target cells expressing two cancer antigens, Daudi cells expressing CD19, CD20, and CD22 were labeled with CellTrace Violet. T-cells expressing a CAR targeting CD19 were used for comparison. Labeled target cells were incubated with ACTR-expressing T-cells, at a ratio of 1:1 effector cells (ACTR-expressing T-cells) to target cells (Daudi cells) with an anti-CD19 antibody, anti-CD20, or anti-CD22 antibody. After a 48 hour incubation, cytotoxicity was determined by quantifying the live cells (CellTrace Violet-positive cells) by flow cytometry. The percent cytotoxicity was calculated as the percentage of viable Daudi cells incubated with the ACTR-expressing T cells and one or more antibodies relative to control samples containing no ACTR-expressing T-cells. As shown in FIG. 27, ACTR T-cells combined with CD19-, CD20-, or CD22-targeting antibodies exhibited cytotoxic activity.

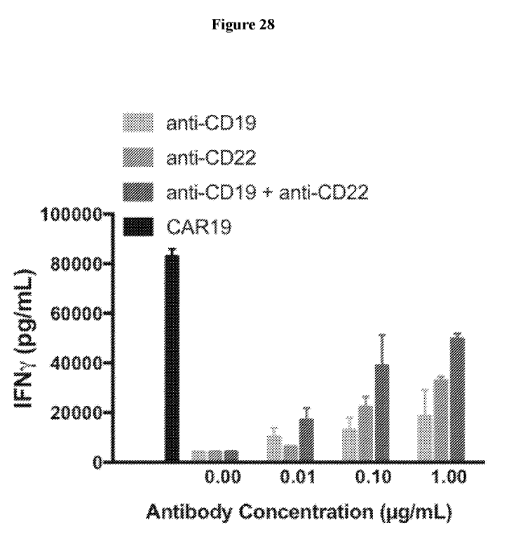

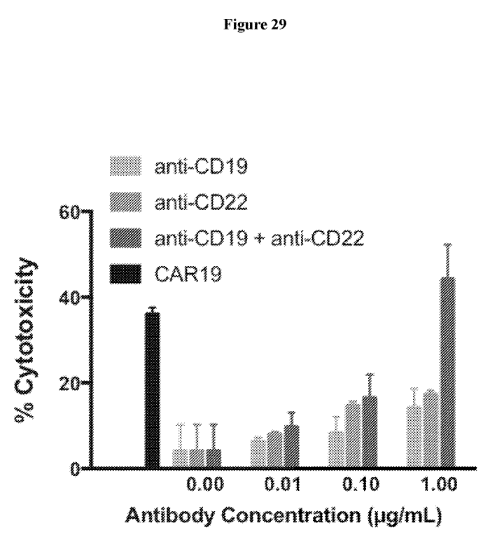

ACTR T-cells were also co-incubated with the RL cell line in the presence of anti-CD19 or anti-CD22 antibodies (separately) or with anti-CD19 and anti-CD22 antibodies (combined) at concentrations of 0, 0.01, 0.10, or 1.00 μg/mL. T-cells expressing CAR19 were used for comparison. The ACTR T-cell production of IFNγ in the various conditions is shown in FIG. 28. The cytotoxicity in the RL target cell lines at the various conditions is shown in FIG. 29. These results demonstrate that combinatorial antigen targeting significantly enhanced ACTR activity.

### Example 6: Sequential Targeting of Tumor Antigens Using ACTR T-Cells

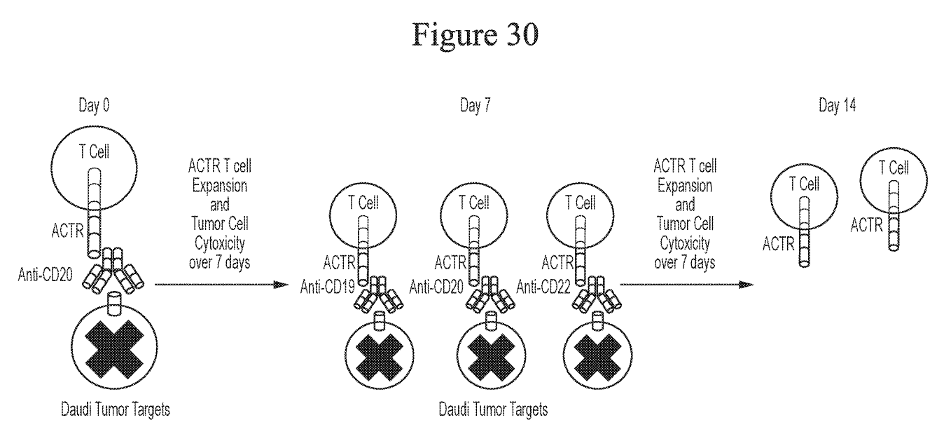

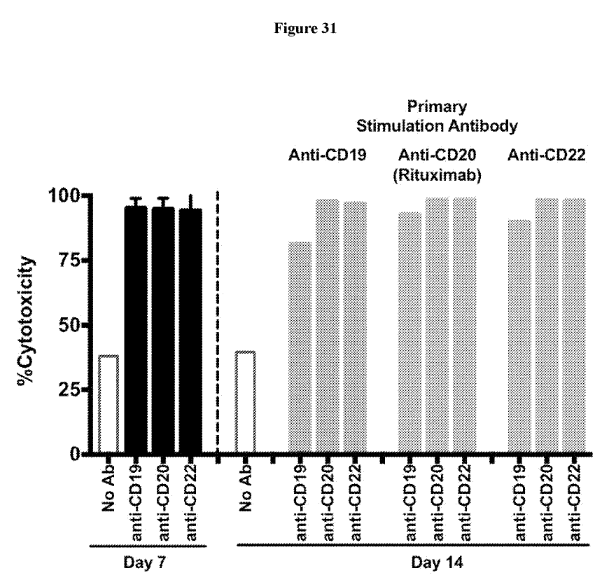

FIG. 30 is an exemplary schematic for a sequential ACTR targeting assay. ACTR T-cells (expression the construct of SEQ ID NO: 1) were co-cultured with Daudi target cells in the presence of anti-CD19, -CD20, or -CD22 antibodies. Seven days after T-cell expansion, the cytotoxicity of Daudi cells was measured by flow cytometry. ACTR T-cells were then washed out. The same ACTR T-cells were subsequently co-incubated with fresh Daudi target cells and a new, different antibody for an additional 14 days. The cytotoxicity of Daudi target cells was measured by flow cytometry again on Day 14. The cytotoxicity of the Daudi cells on days 7 and 14 in the different conditions are shown in FIG. 31. These results demonstrate that ACTR T-cells are cross-functional through sequential rounds of stimulation.

### Example 7: ACTR T-Cells can Overcome Antigen Escape

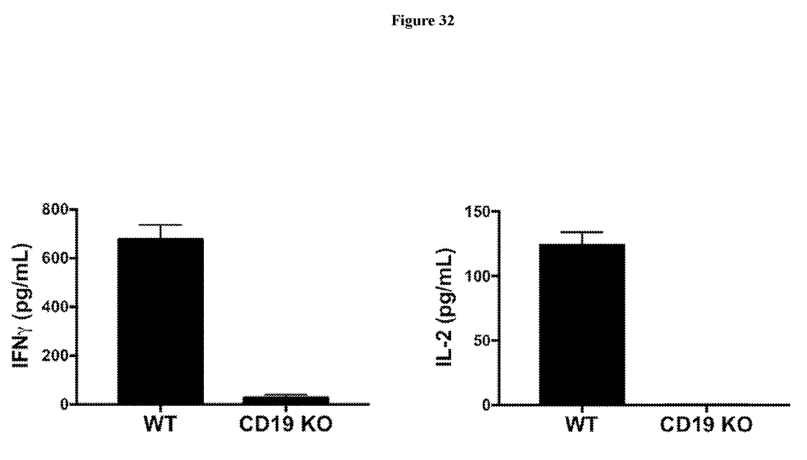

T-cells expressing a CD19 targeting CAR were incubated with wild-type (WT) Raji or CD19 CRISPR knockout Raji (CD19KO) target cells at a ratio of 1:1 (effector cells/ACTR707-expressing T cells to target cells). Supernatants were then analyzed for IFNγ and IL-2 using the Meso Scale Discovery V-Plex Proinflammatory Panel 1 Kit (Human, K15049D-1) following a 24 hour incubation. The Proinflammatory Calibrator Blend, Detection Antibody Solution, and Read Buffer were prepared according to manufacturer protocol. Co-culture supernatants were diluted 1:200 in RPMI 164015 media supplemented with 10% fetal bovine serum. 50 μL of diluted sample or calibrator was added to the MSD plate, and the plate was sealed and incubated on a shaker at 600×g for 2 hours in the dark. The plate was washed three times with 150 μL Phosphate Buffered Saline containing 0.05% Tween-20. 25 μL of detection antibody solution was added to the plate, and the plate was sealed and incubated on a shaker at 600×g for 2 hours in the dark. The plate was washed three times with 150 μL Phosphate Buffered Saline containing 0.05% Tween-20. Read Buffer (150 μL) was added to the plate and the plates were run on the MSD Quickplex SQ 120. The results of these experiments (shown in FIG. 32) demonstrate that T-cells expressing a CD19 targeting CAR are incapable of activation against CD19KO target cells.

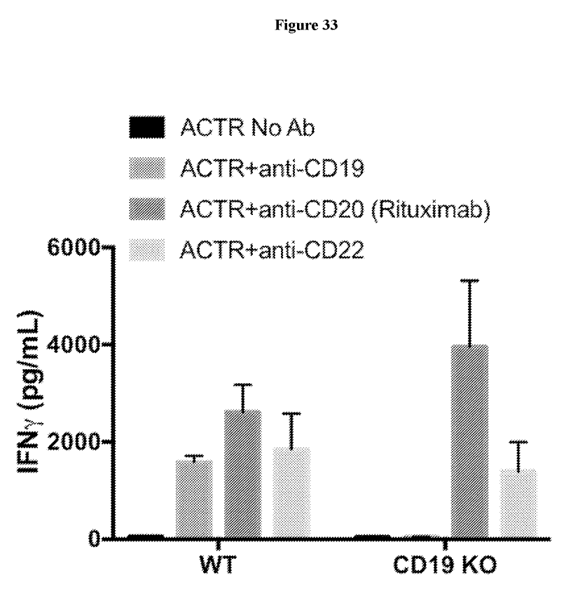

Gamma-retrovirus encoding an ACTR construct (e.g., SEQ ID NO: 67, a.k.a. ACTR707) was generated via routine recombinant technology and used to infect primary human T-cells for generating cells that express ACTR on their cell surface. T-cells expressing ACTR and wild-type (WT) Raji or CD19 CRISPR knockout Raji (CD19KO) target cells were incubated together at a ratio of 1:1 (effector cells/ACTR-expressing T cells to target cells) with anti-CD19, anti-CD20, or anti-CD22 antibodies. Supernatants were subsequently analyzed for IFNγ as above using the Meso Scale Discovery V-Plex Proinflammatory Panel 1 Kit (Human, K15049D-1) following a 24 hour incubation. The levels of IFNγ (in pg/mL) in wild-type (WT) Raji or CD19 CRISPR knockout Raji (CD19KO) target cells that have been incubated with ACTR T-cells alone and in combination with anti-CD19, anti-CD20, or anti-CD22 antibodies are shown in FIG. 33. These results demonstrate that ACTR T-cells are able to effectively generate cytokines targeting CD19KO Raji cells in the presence of anti-CD20 and CD22 antibodies.

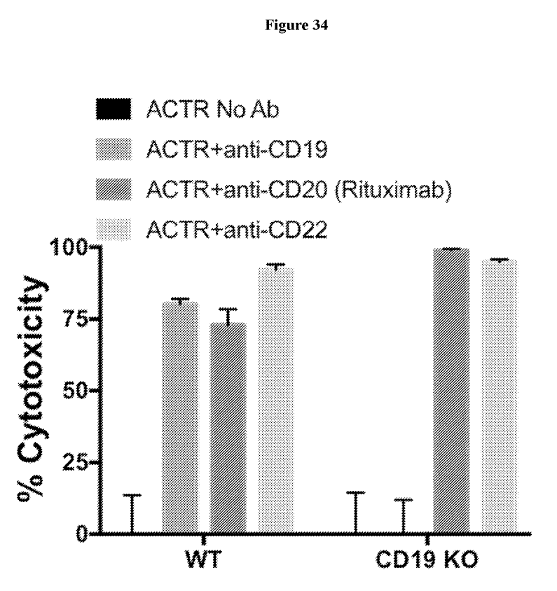

To assess activation and effector function of ACTR-T cells in the presence of wild-type (WT) Raji or CD19 CRISPR knockout Raji (CD19KO) target cells were labeled with CellTrace Violet. Labeled target cells were incubated with ACTR-expressing T cells, at a ratio of 1:1 effector cells (ACTR-expressing T cells) to target cells with the anti-CD19 antibody, anti-CD20, or the anti-CD22 antibody. After incubating for 48 hours, the cytotoxicity was determined by quantifying the live cells (CellTrace Violet-positive cells) by flow cytometry. The percent cytotoxicity was calculated as the percentage of WT or CD19KO cells remaining relative to control samples no containing T cells-expressing ACTR. ACTR T cells combined are able to effectively kill CD19KO Raji cells in the presence of anti-CD20 and CD22 antibodies. The results of the cytotoxicity experiments are shown in FIG. 34 and demonstrate that ACTR T-cells are able to effectively kill CD19KO Raji cells in the presence of anti-CD20 and CD22 antibodies.

### Example 8: Efficient Targeting of HER-2 Non-Amplified Cancer Cells by Antibody-Coupled T Cell Receptor (ACTR) Engineered Autologous T Cells

T-cells expressing an alternative ACTR construct (SEQ ID NO: 67, a.k.a., ACTR707) were shown to mediate potent activity against both HER-2 amplified and HER-2 non-amplified tumor cells in the presence of a combination of HER-2 targeting antibodies trastuzumab, pertuzumab, or both. In particular, it was observed that the co-use of ACTR-T cells and the combination of the two anti-HER2 antibodies induced cytotoxicity against HER2 non-amplified tumor cells, IL-2 production, and T cell proliferation when co-incubated with the HER2 non-amplified tumor cells.

(i) HER2 Expression in Various Tumor Cells

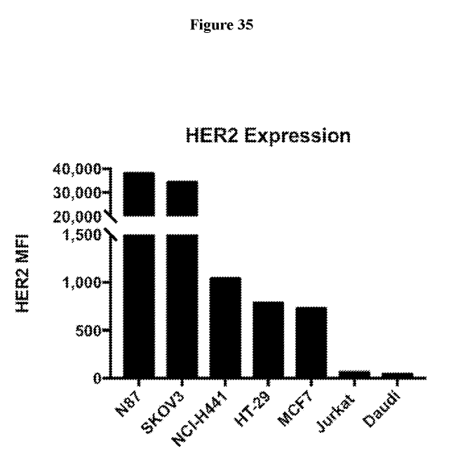

A number of tumor cell lines grown in cell culture were harvested, and were then stained with a fluorescent-conjugated HER2-specific antibody. Fluorescence was measured by flow cytometry (Attune Life Technologies). The values reported are the Mean Fluorescent Intensity values of the staining. HER2-targeting antibodies bind HER2+ cells (N87, SKOV3, NCI-H441, HT-29, and MCF7) and do not bind HER2-Jurkat or Daudi cells. FIG. 35. HER2 mRNA expression and copy number values were derived from public databases (cancer cell line encyclopedia, CCLE). See also Table 4 below.

TABLE 4
HER2 Levels in Various Cancer Cell Lines

| | | Protein | mRNA | Copy No. |
|---|---|---|---|---|
| Cell line | Indication | (FACS MFI) | (CCLE) | (CCLE) |
| N87 | gastric | 37,941 | 13.8 | 4 |
| SKOV3 | ovarian | 34,240 | 11.78 | 1.8 |
| NCI-H441 | lung | 1,042 | 8.5 | 0.2 |
| HT-29 | breast | 786 | 8.11 | 0 |
| MCF7 | breast | 729 | 8.16 | −0.96 |
| Jurkat | T cell | 60 | 5.8 | 0.1 |
| Daudi | B cell | 43 | 6.16 | 0 |

As shown in Table 4, tumor cell lines N87 and SKOV3 express high levels of HER2 and tumor cell lines NCI-H441, HT-29, and MCF7 express low levels of HER2, while HER2 expression in Jurkat and Daudi cells can be deemed as negative.

(ii) Effect of ACTR-T/Trastuzumab Combination on Gastric Cancer N87 Cells

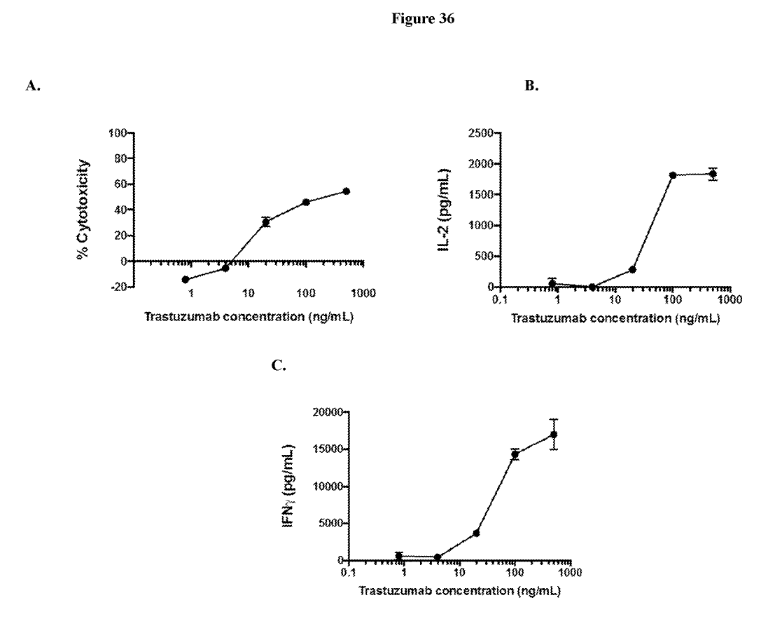

Gamma-retrovirus encoding an exemplary ACTR expression construct SEQ ID NO: 67 was generated via routine recombinant technology and used to infect primary human T-cells to generate cells that express the ACTR construct of SEQ ID NO: 67 on their cell surface. T-cells expressing this ACTR construct and HER2-amplified N87 cells were incubated together at a ratio of 2:1 (effector cells to target cells) with varying concentrations of the HER2-targeting antibody trastuzumab. After incubating for 48 hours, ATPlite reagent (Perkin Elmer) was added to lyse the cells, and the cytotoxicity was determined by quantifying the ATP content of live target cells (remaining luciferase signal). Luminescence was measured using an EnVision Multi-label plate reader (Perkin-Elmer; Waltham, Mass.). The percent cytotoxicity was calculated as the amount of signal from remaining target cells relative to control samples. The ACTR-T cells in combination with trastuzumab exhibited cytotoxic activity on N87 tumor cells. FIG. 36, panel A. Supernatants were analyzed for IL-2 and IFNg following a 24 hour incubation using a CisBio immunoassay according to the manufacturer's protocol. The ACTR-T cells in combination with trastuzumab released IL-2 and IFNg in the presence of N87 tumor cells. FIG. 36, panels B and C.

(iii) Effect of ACTR-T/Trastuzumab Combination on Gastric Cancer Xenograft Mice

HER2-expressing N87 gastric cancer cells were inoculated subcutaneously in female NSG mice. Seven days later mice were randomized based on tumor volume and assigned to the following treatment groups: vehicle, 100 μg trastuzumab, T cells expressing ACTR construct of SEQ ID NO: 67, and the ACTR-T cells in combination with trastuzumab. Trastuzumab was administered by intraperitoneal injection once per week for four weeks starting on Day 7. ACTR707 T cells (1.5×10⁷ total T cells) were administered intravenously once per week for two weeks starting on Day 8. Tumor volume and body weight were recorded twice per week.

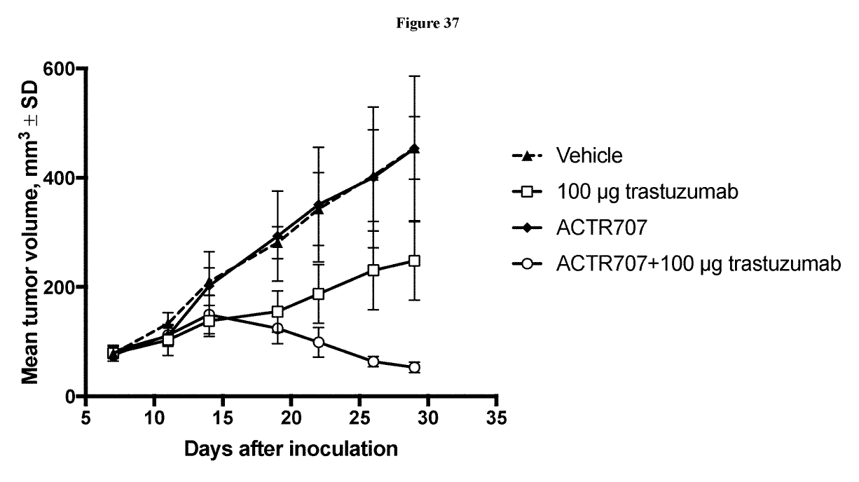

As shown in FIG. 37, the ACTR-T cells in combination with trastuzumab exhibited anti-tumor activity on N87 tumors.

(iv) Cytotoxicity Effect of ACTR-T Cells on HER2 Non-Amplified Cancer Cells in Combination with Trastuzumab or Pertuzumab Alone or in Combination with Both Antibodies

Gamma-retrovirus encoding an exemplary ACTR expression construct of SEQ ID NO: 67 was generated via routine recombinant technology and used to infect primary human T-cells for generating cells that express the ACTR construct on their cell surface. T-cells expressing the ACTR construct and HER2-positive target cells were incubated together at a ratio of 2:1 (effector cells/ACTR-expressing T cells to target cells) with varying concentrations of HER2-targeting antibodies pertuzumab, trastuzumab, or both antibodies in combination. After incubating for 48 hours, ATPlite reagent (Perkin Elmer) was added to lyse the cells, and the cytotoxicity was determined by quantifying the ATP content of live target cells (remaining luciferase signal). Reactions were incubated and luminescence was measured using an EnVision Multi-label plate reader (Perkin-Elmer; Waltham, Mass.). The percent cytotoxicity was calculated as the amount of signal from remaining target cells relative to control samples.

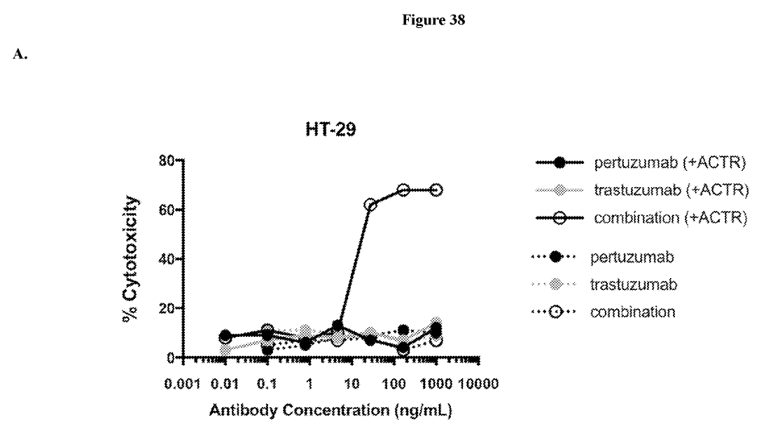

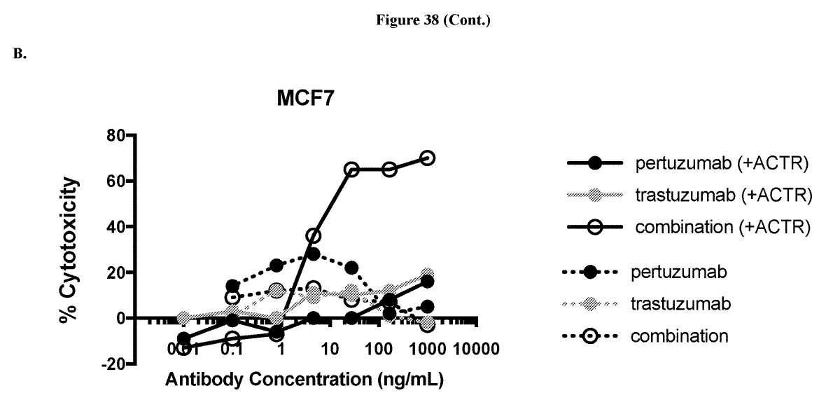

The ACTR-T cells exhibited cytotoxic activity on HER2 non-amplified tumor cells (HT-29 and MCF-7 cells) in combination with trastuzumab and pertuzumab, while no cytotoxic activity of the ACTR-T cells was observed with either trastuzumab or pertuzumab alone. FIG. 38, panels A and B.

(v) IL-2 Production Effect of ACTR-T Cells on HER2 Non-Amplified Cancer Cells in Combination with Trastuzumab or Pertuzumab Alone or in Combination with Both Antibodies

Gamma-retrovirus encoding an exemplary ACTR expression construct of SEQ ID NO: 67 was generated via routine recombinant technology and used to infect primary human T-cells for generating cells that express the ACTR construct on their cell surface. T-cells expressing the ACTR construct and HER2-positive target cells were incubated together at a ratio of 2:1 (effector cells/ACTR-expressing T cells to target cells) with varying concentrations of HER2-targeting antibodies pertuzumab, trastuzumab, or both antibodies in combination. Supernatants were analyzed for IL-2 using following a 24 hour incubation using a CisBio immunoassay according to manufacturer protocol.

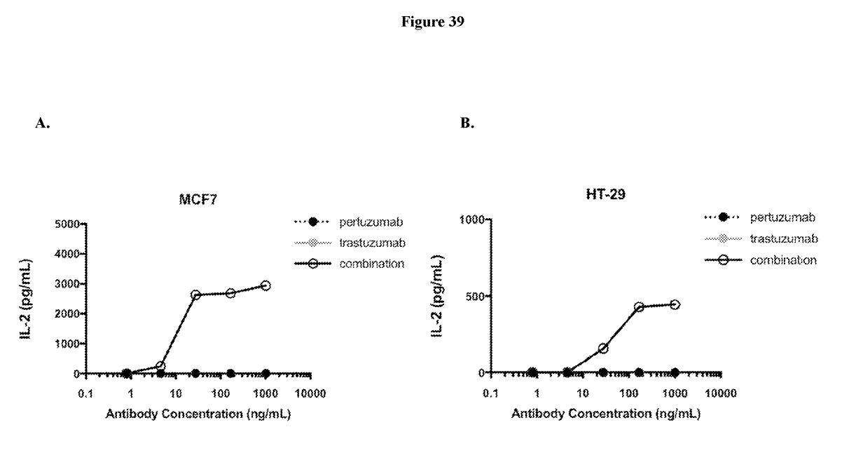

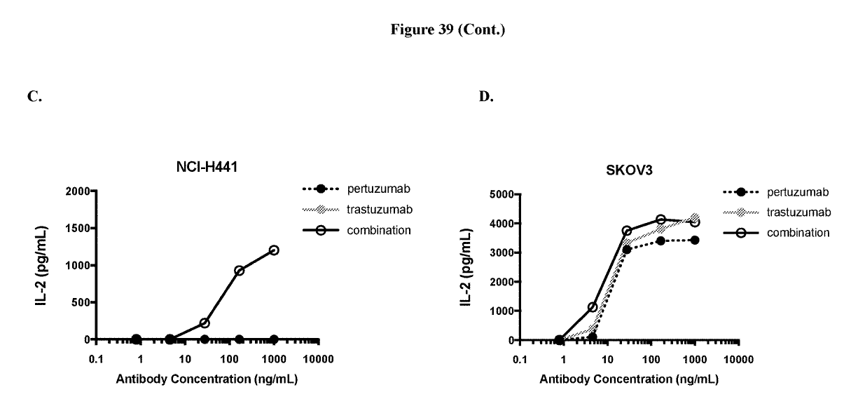

The ACTR-T cells released IL-2 on HER2 non-amplified tumor cells in combination with trastuzumab and pertuzumab, while no IL-2 release from ACTR707 T cells was observed with either trastuzumab or pertuzumab alone. FIG. 39, panels A, B, and C. The ACTR-T cells released IL-2 in combination with either trastuzumab or pertuzumab, and the combination of both antibodies, in the presence of high HER2-expressing line SKOV3. FIG. 39, panel D.

(vi) Proliferation of ACTR-T Cells in Combination with Trastuzumab or Pertuzumab Alone or in Combination with Both in the Presence of HER2-Non Amplified Cancer Cells

The antibody and target-specific proliferation of the ACTR-T cells noted above in the presence of target and HER2-targeting antibodies was determined in a seven-day flow-based proliferation assay. T-cells expressing the ACTR construct of SEQ ID NO: 67 and HER2-positive target cells were incubated together at a ratio of 2:1 (effector cells/ACTR-expressing T cells to target cells) with varying concentrations of HER2-targeting antibodies pertuzumab, trastuzumab, or both antibodies in combination. The number of T-cells was determined by flow cytometry on Day 7 with an anti-human CD3 antibody.

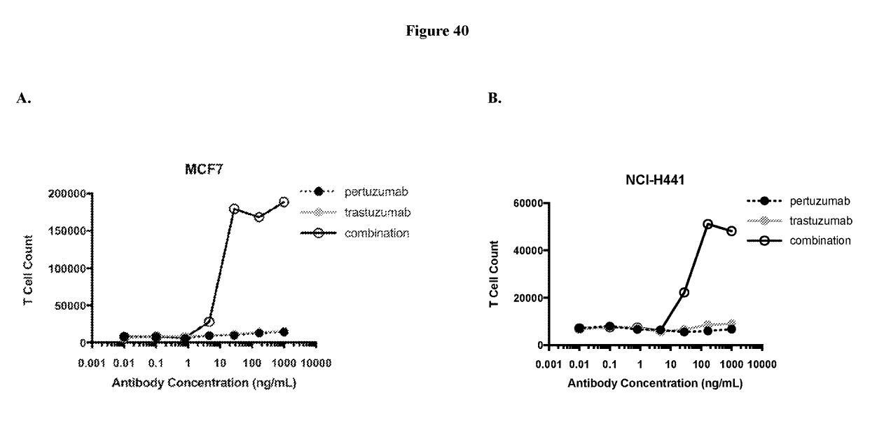

The ACTR-T cells proliferated in the presence of HER2 non-amplified tumor cells in combination with trastuzumab and pertuzumab, while no proliferative activity of the ACTR-T cells was observed with either trastuzumab or pertuzumab alone. FIG. 40.

In sum, results of this study showed that T cells expressing an exemplary ACTR construct (SEQ ID NO: 67) combined with trastuzumab targeted HER2-amplified tumor cells in vitro (indicated by cytotoxicity and IL-2 and IFNγ production) and in vivo. Further, the results indicated that combining ACTR-T cells with multiple anti-HER2 antibodies (here the combination of trastuzumab and pertuzumab) targeted HER2 non-amplified cancer cells as indicated by cytotoxicity, IL-2 production, and T cell proliferation, while such activities were not observed when the ACTR-T cells were in combination with one anti-HER2 antibody (either trastuzumab or pertuzumab). These results demonstrated the superior therapeutic effects of combining the ACTR technology with multiple antibodies targeting a tumor antigen.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of treating cancer in a subject, the method comprising administering to a subject in need thereof:
(i) a therapeutically effective amount of an immune cell that expresses a surface chimeric receptor, wherein the chimeric receptor comprises:
(a) an Fc binding domain, and
(b) a cytoplasmic signaling domain; and
(ii) a therapeutically effective amount of at least two anti-cancer antibodies.

2. The method of claim 1, wherein the chimeric receptor further comprises one or more additional domains selected from the group consisting of:
a transmembrane domain;
a co-stimulatory signaling domain; and
a hinge domain.

3. The method of claim 1, wherein the chimeric receptor comprises, from N-terminus to C-terminus,
(a) the Fc binding domain,
(b) the transmembrane domain,
(c) the co-stimulatory domain, and
(d) the cytoplasmic signaling domain.

4. The method of claim 3, wherein the chimeric receptor further comprises a hinge domain, which is located between (a) and (b).

5. The method of claim 1, wherein the chimeric receptor further comprises a signal peptide.

6. The method of claim 1, wherein the Fc binding domain is an extracellular domain of an Fc receptor.

7. The method of claim 6, wherein the Fc receptor is a Fcγ receptor (FcγR).

8. The method of claim 7, wherein the FcγR is selected from the group consisting of CD16A, CD16B, CD64A, CD64B, CD64C, CD32A, and CD32B.

9. The method of claim 1, wherein the chimeric receptor comprises a co-stimulatory domain of 4-1BB or CD28.

10. The method of claim 1, wherein the chimeric receptor comprises a cytoplasmic signaling domain of CD3ζ.

11. The method of claim 2, wherein the chimeric receptor comprises an extracellular domain of CD16A, a co-stimulatory domain of 4-1BB or CD28, and a cytoplasmic signaling domain of CD3ζ.

12. The method of claim 11, wherein the chimeric receptor comprises a hinge domain of CD8a or CD28.

13. The method of claim 12, wherein the chimeric receptor comprises the amino acid sequence SEQ ID NO: 1 or SEQ ID NO:67.

14. The method of claim 1, wherein the at least two anti-cancer antibodies bind to two different cancer antigens.

15. The method of claim 1, wherein the at least two anti-cancer antibodies bind to different epitopes of the same cancer antigen.

16. The method of claim 14, wherein the cancer antigen is HER2 or EGFR.

17. The method of claim 16, wherein the cancer antigen is HER2 and the at least two anti-cancer antibodies are Trastuzumab and Pertuzumab.

18. The method of claim 16, wherein the cancer antigen is EGFR and the at least two anti-cancer antibodies are selected from the group consisting of cetuximab, panitumumab, MM-151, SCT200, GC1118, Necitumumab, Nimotuzumab, anti-EGFR antibody 992, anti-EGFR antibody 1024, and Zalutumumab.

19. The method of claim 1, wherein the cancer is of a tissue selected from the group consisting of breast, haematopoeitic cells, large intestine, liver, lung, ovary, salivary gland, skin, stomach, and aerodigestive tract.

20. The method of claim 19, wherein the cancer is of B cell origin.

21. The method of claim 20, the cancer of B cell origin is selected from the group consisting of B-lineage acute lymphoblastic leukemia cells, B-cell chronic lymphocytic leukemia cells, B-cell non-Hodgkin's lymphoma cells, Hairy cell leukemia cells, and multiple myeloma cells.

22. The method of claim 1, wherein the subject is a human cancer patient who has cancer cells that express a low level of the cancer antigen, to which the at least two antibodies target.

23. The method of claim 22, wherein the human cancer patient has cancer cells that express a low level of HER2, EGFR, BCMA, CD19, CD20, CD22, CD38, or CS1.

24. The method of claim 1, wherein one of the at least two anti-cancer antibodies is selected from the group consisting of an anti-BCMA antibody, anti-CD19 antibody, an anti-CD20 antibody, an anti-CD22 antibody, an anti-CS1 antibody, and an anti-CD38 antibody.

25. The method of claim 24, wherein the at least two anti-cancer antibodies are
(i) an anti-CD19 antibody and an anti-CD22 antibody,
(ii) an anti-CD19 antibody and an anti-CD20 antibody,
(iii) an anti-CD20 antibody and an anti-CD22 antibody,
(iv) an anti-CS1 antibody and an anti-CD38 antibody,
(v) an anti-CS1 antibody and an anti-BCMA antibody, or
(vi) an anti-BCMA antibody and an anti-CD38 antibody.

26. The method of claim 22, wherein the human cancer patient has HER2 non-amplified cancer cells and the at least two anti-cancer antibodies are Trastuzumab and Pertuzumab.

27. The method of claim 1, wherein the method further comprises, prior to the administering step, identifying the subject as having cancer cells that are positive of the cancer antigen to which at least one of the two anti-cancer antibodies bind.

28. The method of claim 1, wherein the method further comprises, prior to the administering step, identifying the subject as having cancer cells expressing a lower level of the cancer antigen to which at least one of the two anti-cancer antibodies bind.

29. A kit comprising
(i) immune cells that express a chimeric receptor comprising
(a) an Fc binding domain, and
(b) a cytoplasmic signaling domain; and
(ii) at least two anti-cancer antibodies.

30. The kit of claim 29, wherein the chimeric receptor further comprises one or more additional domains selected from the group consisting of:
a transmembrane domain;
a co-stimulatory signaling domain; and
a hinge domain.

31. The kit of claim 29, wherein the chimeric receptor comprises, from N-terminus to C-terminus,
(a) the Fc binding domain,
(b) the transmembrane domain,
(c) the co-stimulatory domain, and
(d) the cytoplasmic signaling domain.

32. The kit of claim 31, wherein the chimeric receptor further comprises a hinge domain, which is located between (a) and (b).

33. The kit of claim 29, wherein the chimeric receptor further comprises a signal peptide.

34. The kit of claim 29, wherein the Fc binding domain is an extracellular domain of an Fc receptor.

35. The kit of claim 34, wherein the Fc receptor is a Fcγ receptor (FcγR).

36. The kit of claim 35, wherein the FcγR is selected from the group consisting of CD16A, CD16B, CD64A, CD64B, CD64C, CD32A, and CD32B.

37. The kit of claim 29, wherein the chimeric receptor comprises a co-stimulatory domain of 4-1BB or CD28.

38. The kit of claim 29, wherein the chimeric receptor comprises a cytoplasmic signaling domain of CD3ζ.

39. The method of claim 38, wherein the chimeric receptor comprises an extracellular domain of CD16A, a co-stimulatory domain of 4-1BB or CD28, and a cytoplasmic signaling domain of CD3ζ.

40. The method of claim 39, wherein the chimeric receptor comprises a hinge domain of CD8a or CD28.

41. The kit of claim 29, wherein the chimeric receptor comprises the amino acid sequence SEQ ID NO: 1 or SEQ ID NO:67.

42. The kit of claim 29, wherein the at least two anti-cancer antibodies bind to two different cancer antigens.

43. The kit of claim 29, wherein the at least two anti-cancer antibodies bind to different epitopes of the same cancer antigen.

44. The kit of claim 42, wherein the cancer antigen is HER2, EGFR, BCMA, CD19, CD20, CD22, CD38, and/or CS1.

45. The kit of claim 44, wherein one of the at least two anti-cancer antibodies is selected from the group consisting of an anti-BCMA antibody, anti-CD19 antibody, an anti-CD20 antibody, an anti-CD22 antibody, an anti-CD38 antibody, an anti-EGFR antibody, an anti-HER2 antibody, and an anti-CS1 antibody.

46. The kit of claim 45, wherein the cancer antigen is HER2 and the at least two anti-cancer antibodies are Trastuzumab and Pertuzumab.

47. The kit of claim 45, wherein the cancer antigen is EGFR and the at least two anti-cancer antibodies are selected from the group consisting of: cetuximab, panitumumab, MM-151, SCT200, GC1118, Necitumumab, Nimotuzumab, anti-EGFR antibody 992, anti-EGFR antibody 1024, and Zalutumumab.

48. The kit of claim 45, wherein the cancer antigens are CD19 and CD22 and the at least two anti-cancer antibodies are MOR208 and Epratuzumab.

49. The kit of claim 48, wherein the Epratuzumab is afucosylated.

50. The kit of claim 45, wherein the cancer antigens are CD38 and/or CS1.

51. The kit of claim 50, wherein the at least two anti-cancer antibodies is an anti-CD38 antibody and an anti-CS1 antibody.

52. The kit of claim 51, wherein the anti-CD38 antibody is Daratumumab and the anti-CS1 antibody is Elotuzumab.

53. An in vitro method for evaluating the activity of an immune cell that expresses a chimeric receptor, the method comprising:
(a) incubating the immune cell expressing the chimeric receptor with a first target cell in the presence of a first agent that comprises an Fc domain, wherein the chimeric receptor comprises an Fc binding domain, and a cytoplasmic signaling domain; and wherein the first agent binds a surface receptor of the first target cell;
(b) measuring cytotoxicity of the first target cell induced by the immune cell in the presence of the first agent;
(c) separating the immune cell from the first target cell and the first agent;
(d) incubating the immune cell obtained in step (c) with a second target cell in the presence of a second agent that comprises an Fc domain, wherein the second agent binds a surface receptor of the second target cell; and
(e) measuring cytotoxicity of the second target cell induced by the immune cell in the presence of the second agent.

54. The in vitro method of claim 53, wherein the first agent, the second agent, or both are antibodies.

55. The in vitro method of claim 54, wherein the first agent and the second agent are antibodies specific to different cell surface receptors.

56. The method of claim 53, wherein the first target cell, the second target cell, or both are cancer cells.

57. The method of claim 53, wherein the chimeric receptor further comprises a signal domain from a co-stimulatory receptor.


SEQUENCE LISTING (69 sequences)

SEQ ID NO: 1 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 442 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIISFFLALTSTALLFLLFFLTLRFSVVKRGKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 442 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDFWVLVVVGGVLACYSLLVTVAFIIFWVRSKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLIALVTSGALLAVLGITGYFLMNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLLAALLALLAALLALLAALLARSKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 428 aa, Artificial Sequence
  Synthetic Polypeptide
MALPVTALLLPLALLLHAARPQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 435 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCALYLLRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 477 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 391 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTSESATPESGPGTSTEPSEGSAPGSPAGSPTIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 435 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 477 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRGGHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 437 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDMALIVLGGVAGLLLFIGLGIFFCVRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDMALIVLGGVAGLLLFIGLGIFFCVRRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLCYILDAILFLYGIVLTLLYCRLKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLLLILLGVLAGVLATLAALLARSKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDITLGLLVAGVLVLLVSLGVAIHLCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVSFCLVMVLLFAVDTGLYFSVKTNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVAAILGLGLVLGLLGPLAILLALYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLCYLLDGILFIYGVILTALFLRVKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 441 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVMSVATIVIVDICITGGLLLLVYYWSKNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDGFLFAEIVSIFVLAVGVYFIAGQDKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 437 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDGIIVTDVIATLLLALGVFCFAGHETKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 435 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVIGFRILLLKVAGFNLLMTLRLWKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 438 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIIVAVVIATAVAAIVAAVVALIYCRKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVLFYLAVGIMFLVNTVLWVTIRKEKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIIILVGTAVIAMFFWLLLVIILRTKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 432 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLGWLCLLLLPIPLIVWVKRKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIAIYCIGVFLIACMVVTVILCRMKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 476 aa, Artificial Sequence
  Synthetic Polypeptide
MALPVTALLLPLALLLHAARPQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 623 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 510 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQEPKSCDKTHTCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 403 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQEPKSCDKTHTCPIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 421 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEAFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 406 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 406 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 421 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSGGGGSGGGGSGGGGSGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 451 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSGGGGSGGGGSGGGGSGGGGSGGGGSGGGGSGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 451 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTSESATPESGPGTSTEPSEGSAPGSPAGSPTSTEEGTSTEPSEGSAIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 421 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTSESATPESGPGTSTEIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 406 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 433 aa, Artificial Sequence
  Synthetic Polypeptide
MLRLLLALNLFPSIQVTGGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 436 aa, Artificial Sequence
  Synthetic Polypeptide
METDTLLLWVLLLWVPGSTGDGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 431 aa, Artificial Sequence
  Synthetic Polypeptide
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 432 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 442 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCQRRKYRSNKGESPVEPAEPCRYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 452 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCQLGLHIWQLRSQCMWPRETQLLLEVPPSTEDARSCQFPEEERGERSAEEKGRLGDLWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 454 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCCVKRRKPRGDVVKVIVSVQRKRQEAEGEATVIEALQAPPDVTTVAVEETIPSFTGRSPNHRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 442 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKKYFFKKEVQQLSVSFSSLQIKALQNAVEKEVQAEDNIYIENSLYATDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 453 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCYKVGFFKRNLKEKMEAGRGVPNGIPAEDSEQLASGQEAGDPGCLKPLHEKDSESGGGKDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 510 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 365 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDPQLCYILDAILFLYGIVLTLLYCRLKIQVRKAAITSYEKSDGVYTGLSTRNQETYETLKHEKPPQKRGRKKLLYIFKQPFMRPVQTTQEEDGCSCRFPEEEEGGCEL

SEQ ID NO: 56 - protein, 366 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRLKIQVRKAAITSYEKSDGVYTGLSTRNQETYETLKHEKPPQ

SEQ ID NO: 57 - protein, 254 aa, Artificial Sequence
  Synthetic Polypeptide
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 58 - protein, 237 aa, Artificial Sequence
  Synthetic Polypeptide
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 59 - protein, 300 aa, Artificial Sequence
  Synthetic Polypeptide
  MISC_FEATURE (16)..(300): May be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 60 - protein, 15 aa, Artificial Sequence
  Synthetic Polypeptide
GGGGSGGGGSGGGGS

SEQ ID NO: 61 - protein, 30 aa, Artificial Sequence
  Synthetic Polypeptide
GGGGSGGGGSGGGGSGGGGSGGGGSGGGGS

SEQ ID NO: 62 - protein, 60 aa, Artificial Sequence
  Synthetic Polypeptide
GGGGSGGGGSGGGGSGGGGSGGGGSGGGGSGGGGSGGGGSGGGGSGGGGSGGGGSGGGGS

SEQ ID NO: 63 - protein, 75 aa, Artificial Sequence
  Synthetic Polypeptide
GGGGSGGGGSGGGGSGGGGSGGGGSGGGGSGGGGSGGGGSGGGGSGGGGSGGGGSGGGGSGGGGSGGGGSGGGGS

SEQ ID NO: 64 - protein, 150 aa, Artificial Sequence
  Synthetic Polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 65 - protein, 225 aa, Artificial Sequence
  Synthetic Polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 66 - protein, 300 aa, Artificial Sequence; identical to SEQ ID NO: 59
  Synthetic Polypeptide

SEQ ID NO: 67 - protein, 432 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 6 aa, Artificial Sequence
  Synthetic Polypeptide
  misc_feature (1)..(1): Xaa is Ser, Ile, Val, or Leu
  misc_feature (2)..(2): Xaa can be any naturally occurring amino acid
  misc_feature (4)..(5): Xaa can be any naturally occurring amino acid
  misc_feature (6)..(6): Xaa is Val, Leu, or Ile
XXYXXX

SEQ ID NO: 69 - protein, 16 aa, Artificial Sequence
  Synthetic Polypeptide
  misc_feature (2)..(3): Xaa can be any naturally occurring amino acid
  misc_feature (4)..(4): Xaa is Leu or Ile
  misc_feature (5)..(12): Xaa can be any naturally occurring amino acid
  misc_feature (11)..(12): May be absent
  misc_feature (14)..(15): Xaa can be any naturally occurring amino acid
  misc_feature (16)..(16): Xaa is Leu or Ile
YXXXXXXXXXXXYXXX